(19) United States
(12) Patent Application Publication
(10) Pub. No.: US 2026/0234135 A1
(43) Pub. Date: Aug. 13, 2026

(54) CRYSTALLINE FORMS OR SALTS OF A DEUTERATED COMPOUND

(71) Applicant: ALEBUND PHARMACEUTICALS (HONG KONG) LIMITED, Hong Kong (CN)

(72) Inventors: Fang LI, Hong Kong (CN); Jialiang LI, Hong Kong (CN); Chutian SHU, Hong Kong (CN)

(21) Appl. No.: 19/155,557

(22) PCT Filed: Feb. 4, 2024
(86) PCT No.: PCT/CN2024/075822

§ 371 Date: Aug. 12, 2025

(30) Foreign Application Priority Data
Feb. 13, 2023 (WO) PCT/CN2023/075624

(51) Int. Cl. C07D 413/12; A61K 31/422; A61P 3/06; A61P 3/10; A61P 13/12; C07B 59/00; C07C 215/10; C07C 279/14

(52) U.S. Cl. CPC C07D 413/12; A61K 31/422; A61P 3/06; A61P 3/10; A61P 13/12; C07B 59/002; C07C 215/10; C07C 279/14

(57) ABSTRACT

The present disclosure relates to various crystalline forms of Compound 2 or salt of Compound 2, as well as pharmaceutical compositions, methods of making and methods of using the same. These crystalline forms are useful in the treatment of diseases and disorders which are modulated by PPARα and/or PPARγ agonists.

### FIELD OF THE DISCLOSURE

The present disclosure generally relates to crystalline forms of Compound 2 or salt of Compound 2, as well as pharmaceutical compositions comprising these crystalline forms or salts and methods of treatment by administration of these crystalline forms or salts or the pharmaceutical compositions.

### BACKGROUND OF THE DISCLOSURE

Peroxisome Proliferator Activated Receptors (PPAR's) are members of the nuclear hormone receptor super family, which are ligand-activated transcription factors regulating gene expression. Various subtypes thereof have been identified and cloned. These include PPARα, PPARβ (also known as PPARδ), and PPARγ. At least two major isoforms of PPARγ exist. While PPARγ1 is ubiquitously expressed in most tissues, the longer isoform PPARγ2 is almost exclusively found in adipocytes. In contrast, PPARα is predominantly expressed in the liver, kidney and heart. PPAR's modulate a variety of body responses including glucose- and lipid-homeostasis, cell differentiation, inflammatory responses and cardiovascular events. It is of great significance for clinical drug development to combine the lipid metabolism regulation activity of PPARα and the insulin sensitivity regulation activity of PPARγ to develop specific PPAR-α/γ dual agonists to control blood sugar and improve cardiovascular symptoms.

As a PPAR-α/γ dual agonist, aleglitazar has a relatively balanced PPAR-α/γ activity. Aleglitazar can effectively improve fasting and postprandial blood sugar levels, insulin sensitivity and blood lipid parameters. However, the results of phase 3 clinical trials of aleglitazar showed that although it can effectively reduce blood sugar and blood lipid levels, it also brings a certain degree of heart failure risk, so it does not produce corresponding cardiovascular benefits. At the same time, adverse reactions such as fracture risk have also been found in clinical trial, and it's known that these adverse reactions are caused by PPARγ activation (Lincoff A M, et al. JAMA. 2014; 311 (15): 1515-1525). Therefore, it is necessary to develop suitable selective PPAR-α/γ dual agonists to improve safety and benefit patients.

The present disclosure provides a novel dual agonist of PPARα and PPARγ by deuterating aleglitazar, i.e., Compound 2:

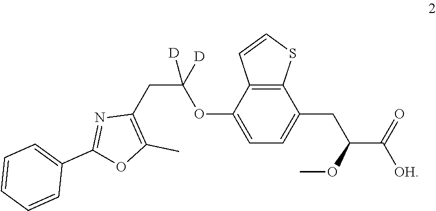 [SMILES: [2H]C([2H])(Cc1nc(-c2ccccc2)oc1C)Oc1ccc(C[C@H](OC)C(=O)O)c2sccc12]

Compound 2 has PPAR-α/γ selectivity different from aleglitazar. Compound 2 has a stronger PPARα activity, and thus can display better PPARα activity in in vitro transcription and in vivo lipid reduction, while maintaining a certain degree of PPARγ activity. Therefore, it can still have good therapeutic effects in regulating the levels of blood lipid and blood sugar, and meanwhile can reduce the side effects caused by PPARγ activity, such as weight gain and the risk of heart failure. Compared with aleglitazar, Compound 2 has a more reasonable risk-benefit ratio for patients suffering from metabolic syndrome, and has good clinical application prospects.

Many compounds can exist in different crystalline forms, or polymorphs, which exhibit different physical, chemical, and spectroscopic properties. In certain case of drugs, certain solid forms may be more bioavailable than others, while others may be more stable under manufacturing, storage, and biological conditions. In order to ensure the quality, safety and efficacy of the drug during manufacture, it is important to have one polymorph of a compound that exhibits certain desirable physical and chemical (including spectroscopic) properties.

Accordingly, there is a strong need for one or more crystalline forms of Compound 2 or salts of Compound 2 that have an acceptable balance of these properties.

### SUMMARY OF THE DISCLOSURE

The present disclosure provides crystalline forms of Compound 2 or a salt of Compound 2:

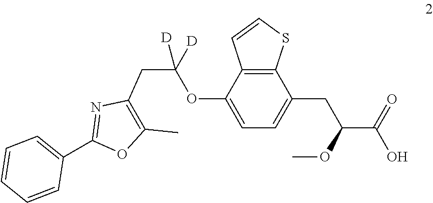 [SMILES: [2H]C([2H])(Cc1nc(-c2ccccc2)oc1C)Oc1ccc(C[C@H](OC)C(=O)O)c2sccc12]

including several anhydrous, hydrate and solvate forms, and solid state forms thereof, pharmaceutical compositions, which are capable of modulating PPARα and PPARγ. Methods for use of such compounds for treatment of various diseases or conditions, such as diabetes, are also provided.

In one aspect, the present disclosure provides a crystalline form B of Compound 2, characterized by an XRPD pattern comprising one or more peaks at 8.16, 13.23 and 13.90 (±0.2° 2θ).

In another aspect, the present disclosure provides a crystalline form C of Compound 2, characterized by an XRPD pattern comprising one or more peaks at 5.06, 10.09 and 20.24 (±0.2° 2θ).

In a further aspect, the present disclosure provides a crystalline form D of Compound 2, characterized by an XRPD pattern comprising one or more peaks at 5.03, 12.98, 15.91 and 21.58 (±0.2° 2θ).

In a further aspect, the present disclosure provides a crystalline form E of Compound 2, characterized by an XRPD pattern comprising one or more peaks at 10.12, 11.97, 13.73 and 18.89 (±0.2° 2θ).

In a further aspect, the present disclosure provides a crystalline form F of Compound 2, characterized by an XRPD pattern comprising one or two peaks at 15.50 and 21.23 (±0.2° 2θ).

In a further aspect, the present disclosure provides a crystalline form G of the maleate of Compound 2, characterized by an XRPD pattern comprising one or more peaks at 9.67, 11.60, 12.90 and 14.43 (±0.2° 2θ).

In another aspect, the present disclosure provides a salt of Compound 2, wherein the salt of Compound 2 is selected from sodium salt, potassium salt, arginine salt, magnesium salt or tromethamine salt of compound 2.

In some embodiments, the present disclosure provides a crystalline form A of sodium salt of Compound 2, characterized by an XRPD pattern comprising one or more peaks at 4.09, 4.62 and 14.21 (±0.2° 2θ).

In some embodiments, the present disclosure provides a crystalline form C of potassium salt of Compound 2, characterized by an XRPD pattern comprising one or more peaks at 12.91, 14.96 and 21.23 (±0.2° 2θ).

In some embodiments, the present disclosure provides a crystalline form A of arginine salt of Compound 2, characterized by an XRPD pattern comprising one or more peaks at 12.93, 13.43, 18.21 and 19.49 (±0.2° 2θ).

In some embodiments, the present disclosure provides a crystalline form A of potassium salt of Compound 2, characterized by an XRPD pattern substantially as shown in the upper curve of FIG. 19A.

In some embodiments, the present disclosure provides a crystalline form B of potassium salt of Compound 2, characterized by an XRPD pattern substantially as shown in the lower curve of FIG. 19A.

In some embodiments, the present disclosure provides a crystalline form A of magnesium salt of Compound 2, characterized by an XRPD pattern substantially as shown in FIG. 21A.

In some embodiments, the present disclosure provides a crystalline form A of tromethamine salt of Compound 2, characterized by an XRPD pattern substantially as shown in FIG. 22A.

In another aspect, the present disclosure provides a pharmaceutical composition comprising Compound 2 or a salt of Compound 2 and a pharmaceutically acceptable excipient, wherein Compound 2 or the salt of Compound 2 is in a crystalline form of the present disclosure.

In a further aspect, the present disclosure provides a method for the treatment and/or prophylaxis of diseases which are modulated by PPARα and/or PPARγ agonists in a subject, comprising administering an effective amount of the crystalline forms of Compound 2 or the salt of Compound 2 of the present disclosure or the pharmaceutical composition of the present disclosure to the subject.

In a further aspect, the present disclosure provides use of the crystalline forms of Compound 2 or the salt of Compound 2 of the present disclosure or the pharmaceutical composition of the present disclosure in the manufacture of a medicament for the treatment and/or prophylaxis of diseases which are modulated by PPARα and/or PPARγ agonists.

In a further aspect, the present disclosure provides a method modulating PPARα and/or PPARγ in a subject in need thereof, comprising administering an effective amount of the crystalline forms of Compound 2 or the salt of Compound 2 of the present disclosure or the pharmaceutical composition of the present disclosure to the subject.

### BRIEF DESCRIPTION OF THE DRAWINGS

FIG. 1A shows XRPD overlay of starting material (818159-01-A). FIG. 1B shows TGA/DSC curves of starting material (818159-01-A).

FIG. 2A shows XRPD overlay of starting material (818159-60-A). FIG. 2B shows TGA/DSC curves of starting material (818159-60-A).

FIG. 3 shows XRPD overlay of free acid crystal forms.

FIG. 4A shows XRPD patterns of free acid Form B (prepared from EtOAc, 818159-08-B, with minor Form E) before and after slurry in CAN. FIG. 4B shows TGA/DSC curves of free acid Form B (prepared from EtOAc, 818159-08-B, with minor Form E). FIG. 4C shows ¹H NMR spectrum of free acid Form B (prepared from EtOAc, 818159-08-B, with minor Form E). FIG. 4D shows VT XRPD of free acid Form B (818159-08-B with minor Form E).

FIG. 5A shows XRPD pattern of free acid Form B (818159-39-B). FIG. 5B shows TGA/DSC curves of free acid Form B (818159-39-B).

FIG. 6A shows XRPD pattern of free acid Form C (818159-30-B). FIG. 6B shows TGA/DSC curves of free acid Form C (818159-30-B). FIG. 6C shows ¹H NMR spectrum of free acid Form C (818159-30-B).

FIG. 7 shows XRPD patterns of free acid Form D (818159-25-A11).

FIG. 8 shows XRPD overlay of different batches free acid Form D.

FIG. 9A shows TGA/DSC curves of free acid Form D (818159-36-B, with minor Form E). FIG. 9B shows ¹H NMR spectrum of free acid Form D (818159-36-B, with minor Form E). FIG. 9C shows XRPD overlay of free acid Form D (818159-36-B, with minor Form E) before and after storage at RT.

FIG. 10 shows XRPD overlay of re-prepared free acid Form D.

FIG. 11A shows TGA/DSC curves of free acid Form D (818159-68-B, with minor Form E). FIG. 11B shows ¹H NMR spectrum of free acid Form D (818159-68-B, with minor Form E). FIG. 11C shows VT-XRPD patterns of free acid Form D (818159-68-B, with minor Form E).

FIG. 12 shows XRPD patterns of free acid Form E (818159-40-A2_30Jul).

FIG. 13A shows XRPD overlay of free acid Form E (818159-32-B, with minor Form B). FIG. 13B shows TGA/DSC curves of free acid Form E (818159-32-B, with minor Form B). FIG. 13C shows ¹H NMR spectrum of free acid Form E (818159-32-B, with minor Form B).

FIG. 14A shows XRPD pattern of free acid Form F (818159-37-B). FIG. 14B shows TGA/DSC curves of free acid Form F (818159-37-B). FIG. 14C shows ¹H NMR spectrum of free acid Form F (818159-37-B). FIG. 14D shows VT-XRPD overlay of free acid Form F (818159-37-B).

FIG. 15A shows XRPD pattern of free acid Form G (818159-41-B). FIG. 15B shows TGA/DSC curves of free acid Form G (818159-41-B). FIG. 15C shows ¹H NMR spectrum of free acid Form G (818159-41-B).

FIG. 16A shows XRPD overlay of residual solids from slurry competition of anhydrate forms (I/V). FIG. 16B shows XRPD overlay of residual solids from slurry competition of anhydrate forms (II/V). FIG. 16C shows XRPD overlay of residual solids from slurry competition of anhydrate forms (III/V). FIG. 16D shows XRPD overlay of residual solids from slurry competition of anhydrate forms (IV/V). FIG. 16E shows XRPD overlay of residual solids from slurry competition of anhydrate forms (V/V).

FIG. 17A shows XRPD overlay of residual solids from slurry competition at different water activities (I/II). FIG. 17B shows XRPD overlay of residual solids from slurry competition at different water activities (II/II).

FIG. 18A shows XRPD pattern of Na salt Form A (818159-03-B1). FIG. 18B shows TGA/DSC curves of Na salt Form A (818159-03-B1). FIG. 18C shows ¹H NMR spectrum of Na salt Form A (818159-03-B1).

FIG. 19A shows XRPD patterns of K salt Form A/B (818159-03-B2/C2). FIG. 19B shows TGA/DSC curves of K salt Form A (818159-03-B2). FIG. 19C shows ¹H NMR spectrum of K salt Form A (818159-03-B2).

FIG. 20A shows XRPD pattern of arginine salt Form A (818159-03-B3). FIG. 20B shows TGA/DSC curves of arginine salt Form A (818159-03-B3). FIG. 20C shows ¹H NMR spectrum of arginine salt Form A (818159-03-B3).

FIG. 21A shows XRPD pattern of Mg salt Form A (818159-03-C5). FIG. 21B shows TGA/DSC curves of Mg salt Form A (818159-03-C5). FIG. 21C shows ¹H NMR spectrum of Mg salt Form A (818159-03-C5).

FIG. 22A shows XRPD pattern of tris salt Form A (818159-03-B10). FIG. 22B shows TGA/DSC curves of tris salt Form A (818159-03-B10). FIG. 22C shows ¹H NMR spectrum of tris salt Form A (818159-03-B10).

FIG. 23 shows XRPD overlay of re-prepared Na salt Form A.

FIG. 24A shows XRPD pattern of Na salt Form A (818159-09-B). FIG. 24B shows TGA/DSC curves of re-prepared Na salt Form A (818159-09-B). FIG. 24C shows ¹H NMR spectrum of re-prepared Na salt Form A (818159-09-B).

FIG. 25 shows XRPD overlay of re-prepared K salt Form C.

FIG. 26A shows XRPD pattern of K salt Form C (818159-10-B). FIG. 26B shows TGA/DSC curves of re-prepared K salt Form C (818159-10-B). FIG. 26C shows ¹H NMR spectrum of re-prepared K salt Form C (818159-10-B).

FIG. 27 shows XRPD overlay of re-prepared arginine salt Form A.

FIG. 28A shows XRPD pattern of Arginine salt Form A (818159-11-B). FIG. 28B shows TGA/DSC curves of re-prepared arginine salt Form A (818159-11-B). FIG. 28C shows ¹H NMR spectrum of re-prepared arginine salt Form A (818159-11-B).

FIG. 29A shows XRPD overlay of residual solids from kinetic solubility starting from free acid Form B in water. FIG. 29B shows XRPD overlay of residual solids from kinetic solubility starting from free acid Form B in SGF. FIG. 29C shows XRPD overlay of residual solids from kinetic solubility starting from free acid Form B in FaSSIF-V2. FIG. 29D shows XRPD overlay of residual solids from kinetic solubility starting from free acid Form B in FeSSIF-V2. FIG. 29E shows XRPD overlay of residual solids from kinetic solubility starting from free acid Form B in pH 1.2 buffer. FIG. 29F shows XRPD overlay of residual solids from kinetic solubility starting from free acid Form B in pH 4.5 buffer. FIG. 29G shows XRPD overlay of residual solids from kinetic solubility starting from free acid Form B in pH 6.8 buffer.

FIG. 30A shows XRPD overlay of residual solids from kinetic solubility starting from K salt Form C in water. FIG. 30B shows XRPD overlay of residual solids from kinetic solubility starting from K salt Form C in SGF. FIG. 30C shows XRPD overlay of residual solids from kinetic solubility starting from K salt Form C in pH 1.2 buffer. FIG. 30D shows XRPD overlay of residual solids from kinetic solubility starting from K salt Form C in pH 4.5 buffer.

FIG. 31A shows XRPD overlay of residual solids from kinetic solubility starting from arginine salt Form A in SGF. FIG. 31B shows XRPD overlay of residual solids from kinetic solubility starting from arginine salt Form A in pH 1.2 buffer. FIG. 31C shows XRPD overlay of residual solids from kinetic solubility starting from arginine salt Form A in pH 4.5 buffer. FIG. 31D shows XRPD pattern of residual solids from kinetic solubility starting from arginine salt Form A in pH 6.8 buffer.

FIG. 32A shows XRPD pattern of residual solids from kinetic solubility starting from Na salt Form A in water. FIG. 32B shows XRPD overlay of residual solids from kinetic solubility starting from Na salt Form A in SGF.

FIG. 33 shows XRPD overlay of residual solids from kinetic solubility starting from free acid Form E (818159-61-B, 24 hours).

FIG. 34 shows XRPD overlay of residual solids from kinetic solubility starting from free acid Form B (818159-71-B, 24 hours).

FIG. 35 shows kinetic solubility curves at 37° C.

FIG. 36 shows kinetic solubility curves of free acid Form B and E.

FIG. 37A shows DVS plot of free acid Form B (818159-08-B). FIG. 37B shows XRPD overlay of free acid Form B (818159-08-B) before and after DVS test.

FIG. 38A shows DVS plot of K salt Form C (818159-10-B). FIG. 38B shows XRPD overlay of K salt Form C (818159-10-B) before and after DVS test.

FIG. 39A shows DVS plot of arginine salt Form A (818159-11-B). FIG. 39B shows XRPD overlay of arginine salt Form A (818159-11-B) before and after DVS test.

FIG. 40A shows DVS plot of Na salt Form A (818159-09-B). FIG. 40B shows XRPD overlay of Na salt Form A (818159-09-B) before and after DVS test.

FIG. 41 shows XRPD overlay for free acid Form B (818159-08-B) before and after solid stability evaluation.

FIG. 42 shows XRPD overlay for free acid Form E (818159-61-B) before and after solid stability evaluation.

FIG. 43 shows DSC curves for free acid Form E (821755-05-A/B) after 8 weeks solid stability evaluation.

FIG. 44 shows XRPD overlay for Na salt Form A (818159-09-B) before and after solid stability evaluation.

FIG. 45 shows XRPD overlay for K salt Form C (818159-10-B) before and after solid stability evaluation.

FIG. 46 shows XRPD overlay for arginine salt Form A (818159-11-B) before and after solid stability evaluation.

FIG. 47 shows HPLC chromatogram of free acid Form B (818159-08-B) before and after solid stability evaluation.

FIG. 48 shows HPLC chromatogram of free acid Form E (818159-61-B) before and after solid stability evaluation.

FIG. 49 shows HPLC chromatogram of Na salt Form A (818159-09-B) before and after solid stability evaluation.

FIG. 50 shows HPLC chromatogram of K salt Form C (818159-10-B) before and after solid stability evaluation.

FIG. 51 shows HPLC chromatogram of arginine salt Form A (818159-11-B) before and after solid stability evaluation.

FIG. 52A shows changes of serum TG in each group. Data were presented as Mean±SD; One way ANOVA by Prism GraphPad; n=6. ****P<0.0001, ***P<0.001, **P<0.001, *P<0.05 vs Vehicle. FIG. 52B shows changes of serum NEFA in each group. Data were presented as Mean±SD; One way ANOVA by Prism GraphPad; n=6. ****P<0.0001 vs Vehicle.

FIG. 53A shows changes in body weight gain of each group of animals compared with Day 0. FIG. 53B shows changes in the difference in weight gain between each group of animals and the vehicle group. (*: The weight gain of the treatment group minus the average weight gain of the vehicle group.)

FIG. 54 shows the effect of the compound on the changes of db/db animal's body weight. Data were presented as Mean±SEM; Two way ANOVA followed by Dunnett test by Prism GraphPad; n=6-9.

FIG. 55A shows the effect of the compounds on serum TG levels in db/db animals on Day 6. Data were presented as Mean±SEM; One way ANOVA followed by Dunnett test by Prism GraphPad; n=6-9. **P<0.01, ***P<0.001, ****P<0.0001 vs Model. FIG. 55B shows the effect of the compounds on serum TG levels in db/db animals on Day 12. Data were presented as Mean±SEM; One way ANOVA followed by Dunnett test by Prism GraphPad; n=6-9. ****P<0.0001 vs Model.

FIG. 56 shows the effect of compounds on random blood glucose of db/db animals. Data were presented as Mean±SEM, n=6-9.

FIG. 57A shows the effect of compounds on oral glucose tolerance of db/db animals. Data were presented as Mean±SEM, n=6-9. FIG. 57B shows the effect of compounds on oral glucose tolerance of db/db animals. Data were presented as Mean±SEM; One way ANOVA followed by Dunnett test by Prism GraphPad; n=6-9. **P<0.01, ***P<0.001, ****P<0.0001 vs model group.

FIG. 58 shows the effect of Compound 2 on urinary albumin excretion.

FIG. 59A-59D shows the effect of Compound 2 on Glomerular and Tubular damage.

FIG. 60 shows the effect of Compound 2 on improvement of renal injury in rat model of unilateral ureteral obstruction.

### DETAILED DESCRIPTION OF THE DISCLOSURE

Reference will now be made in detail to certain embodiments of the present disclosure, examples of which are illustrated in the accompanying structures and formulas. While the present disclosure will be described in conjunction with the enumerated embodiments, it will be understood that they are not intended to limit the present disclosure to those embodiments. On the contrary, the present disclosure is intended to cover all alternatives, modifications, and equivalents, which may be included within the scope of the present disclosure as defined by the claims. One skilled in the art will recognize many methods and materials similar or equivalent to those described herein, which could be used in the practice of the present disclosure. The present disclosure is in no way limited to the methods and materials described. In the event that one or more of the incorporated references and similar materials differs from or contradicts this application, including but not limited to defined terms, term usage, described techniques, or the like, the present disclosure controls. All references, patents, patent applications cited in the present disclosure are hereby incorporated by reference in their entireties.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination. It must be noted that, as used in the specification and the appended claims, the singular forms “a,” “an,” and “the” include plural forms of the same unless the context clearly dictates otherwise. Thus, for example, reference to “a compound” includes a plurality of compounds.

### Definitions

As used herein, the term “aleglitazar”, also known as RG-1439 or RO-0728804, is a dual agonist of peroxisome proliferator-activated receptors α/γ (PPARα/γ) with insulin-sensitizing and glucose-lowering actions and favorable effects on lipid profiles. It is investigated for use in patients with type II diabetes to reduce their risks of cardiovascular mortality and morbidity. “Aleglitazar” has the structure as follows:

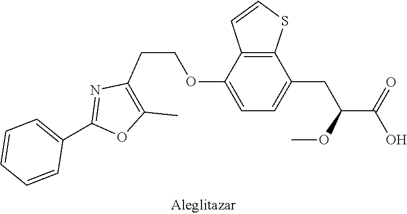 [SMILES: CO[C@@H](Cc1ccc(OCCc2nc(-c3ccccc3)oc2C)c2ccsc12)C(=O)O]

As used herein, the term “Compound 2”, which is a deuterated Aleglitazar, refers to a compound having the following structure:

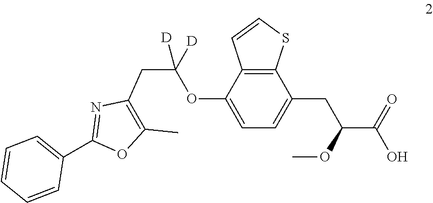 [SMILES: [2H]C([2H])(Cc1nc(-c2ccccc2)oc1C)Oc1ccc(C[C@H](OC)C(=O)O)c2sccc12]

As used herein, the terms “crystal form”, “crystalline form” and “Form” interchangeably refer to a crystal structure (or polymorph) having a particular molecular packing arrangement in the crystal lattice. Crystalline forms can be identified and distinguished from each other by one or more characterization techniques including, for example, X-ray powder diffraction (XRPD), single crystal X-ray diffraction, differential scanning calorimetry (DSC), thermogravimetric analysis (TGA), and/or dynamic vapor sorption (DVS). Accordingly, as used herein, the term “crystalline Form [X] of Compound 2” refers to unique crystalline forms that can be identified and distinguished from each other by one or more characterization techniques including, for example, X-ray powder diffraction (XRPD), single crystal X-ray diffraction, differential scanning calorimetry (DSC), thermogravimetric analysis (TGA), and/or dynamic vapor sorption (DVS). In some embodiments, the novel crystalline forms are characterized by an X-ray powder diffractogram having one or more signals at one or more specified two-theta values (° 2θ).

As used herein, the term “solvate” refers to a crystal form comprising one or more molecules of the compound of the present disclosure and, incorporated into the crystal lattice, one or more molecules of a solvent or solvents in stoichiometric or nonstoichiometric amounts. When the solvent is water, the solvate is referred to as a “hydrate.”

As used herein, the term “XRPD” refers to the analytical characterization method of X-ray powder diffraction. As used herein, the terms “X-ray powder diffractogram”, “X-ray powder diffraction pattern”, “XRPD pattern” interchangeably refer to an experimentally obtained pattern plotting signal positions (on the abscissa) versus signal intensities (on the ordinate). For an amorphous material, an X-ray powder diffractogram may include one or more broad signals; and for a crystalline material, an X-ray powder diffractogram may include one or more signals, each identified by its angular value as measured in degrees 2θ (° 2θ), depicted on the abscissa of an X-ray powder diffractogram.

A “peak” as used herein refers to a point in the XRPD pattern where the intensity as measured in counts is at a local maximum. One of ordinary skill in the art would recognize that one or more signals (or peaks) in an XRPD pattern may overlap and may, for example, not be apparent to the naked eye. Indeed, one of ordinary skill in the art would recognize that some art-recognized methods are capable of and suitable for determining whether a signal exists in a pattern, such as Rietveld refinement.

The repeatability of the measured angular values is in the range of ±0.2° 2θ, i.e., the angular value can be at the recited angular value +0.2 degrees two-theta, the angular value −0.2 degrees two-theta, or any value between those two end points (angular value +0.2 degrees two-theta and angular value −0.2 degrees two-theta). In some embodiments, the repeatability of the measured angular values is in the range of ±0.1° 2θ.

The term “peak intensities” refers to relative signal intensities within a given X-ray powder diffractogram. Factors that can affect the relative signal or peak intensities include sample thickness and preferred orientation (e.g., the crystalline particles are not distributed randomly).

As used herein, an X-ray powder diffractogram is “substantially similar to that in [a particular] Figure” when at least 90%, such as at least 95%, at least 98%, or at least 99%, of the peaks in the two diffractograms overlap. In determining “substantial similarity,” one of ordinary skill in the art will understand that there may be variation in the intensities and/or signal positions in XRPD diffractograms even for the same crystalline form. Thus, those of ordinary skill in the art will understand that the signal maximum values in XRPD diffractograms (in degrees two-theta (° 2θ) referred to herein) generally mean that value is identified as ±0.2 degrees 2θ of the reported value, an art-recognized variance. In some embodiments, the signal variance is identified as ±0.1 degrees 2θ of the reported value.

As used herein, the terms “about” and “substantially” indicate with respect to features such as endotherms, endothermic peak, exotherms, baseline shifts, etc., that their values can vary. With reference to X-ray diffraction peak positions, “about” or “substantially” means that typical peak position and intensity variability are taken into account. For example, one skilled in the art will appreciate that the peak positions (2θ) will show some inter-apparatus variability, typically as much as 0.2°. Occasionally, the variability could be higher than 0.2° depending on apparatus calibration differences. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measure only. For DSC, variation in the temperatures observed will depend upon the rate of temperature change as well as sample preparation technique and the particular instrument employed. Thus, the endotherm/melting point values reported herein relating to DSC/TGA thermograms can vary ±5° C. (and still be considered to be characteristic of the particular crystalline form described herein). When used in the context of other features, such as, for example, percent by weight (% by weight), reaction temperatures, the term “about” indicates a variance of ±5%.

As used herein “amorphous” refers to a solid form of a molecule, atom, and/or ions that is not crystalline. An amorphous solid does not display a definitive X-ray diffraction pattern.

As used herein, “substantially pure,” when used in reference to a form, means a compound having a purity greater than 90 weight %, including greater than 90, 91, 92, 93, 94, 95, 96, 97, 98, and 99 weight %, and also including equal to about 100 weight % of Compound 2, based on the weight of the compound. The remaining material comprises other form(s) of the compound, and/or reaction impurities and/or processing impurities arising from its preparation. For example, a crystalline form of Compound 2 may be deemed substantially pure in that it has a purity greater than 90 weight %, as measured by means that are at this time known and generally accepted in the art, where the remaining less than 10 weight % of material comprises other form(s) of Compound 2 and/or reaction impurities and/or processing impurities.

As used herein, the term “pharmaceutical composition” refers to a formulation containing the compound or crystalline forms thereof provided herein in a form suitable for administration to a subject.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used herein includes both one and more than one such excipient. The term “pharmaceutically acceptable excipient” also encompasses “pharmaceutically acceptable carrier” and “pharmaceutically acceptable diluent”.

As used herein, the term “therapeutically effective amount” refers to an amount of a molecule, compound, or composition comprising the molecule or compound to treat, ameliorate, or prevent an identified disease or condition, or to exhibit a detectable therapeutic or modulatory effect. The effect can be detected by any assay method known in the art. The precise effective amount for a subject will depend upon the subject's body weight, size, and health; the nature and extent of the condition; the rate of administration; the therapeutic or combination of therapeutics selected for administration; and the discretion of the prescribing physician. Therapeutically effective amounts for a given situation can be determined by routine experimentation that is within the skill and judgment of the clinician.

As used herein, a “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In some embodiments, the subject is an experimental animal or animal suitable as a disease model.

### Crystalline Forms

The present disclosure provides crystalline forms of Compound 2 or a salt of Compound 2:

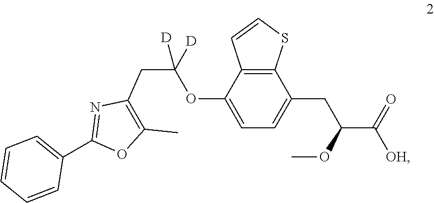 [SMILES: [2H]C([2H])(Cc1nc(-c2ccccc2)oc1C)Oc1ccc(C[C@H](OC)C(=O)O)c2sccc12]

preparation methods for making the compounds, pharmaceutical compositions containing them and various uses of the disclosed compounds.

### Crystalline Form B of Compound 2

In one aspect, the present disclosure relates to a crystalline form B of Compound 2, characterized by an X-ray powder diffractogram (XRPD) pattern comprising one or more peaks at 8.16, 13.23 and 13.90 (±0.2° 2θ). In some embodiments, the XRPD pattern of crystalline form B further comprises one or more peaks at 4.94, 11.83, 14.97, 18.54 and 26.24 (±0.2° 2θ). In some embodiments, the XRPD pattern of crystalline form B further comprises one or more peaks at 9.89, 19.74 and 20.92 (±0.2° 2θ).

In some embodiments, the crystalline form B is characterized by an XRPD pattern comprising one or more peaks selected from the group consisting of:

| °2θ | °2θ | °2θ | °2θ | |
|---|---|---|---|---|
| 4.94 | 13.23 | 18.54 | 22.71 | |
| 8.16 | 13.90 | 19.74 | 25.35 | |
| 9.89 | 14.97 | 20.92 | 26.24 | |
| 11.83 | 16.29 | 21.63 | 28.55 | ; |

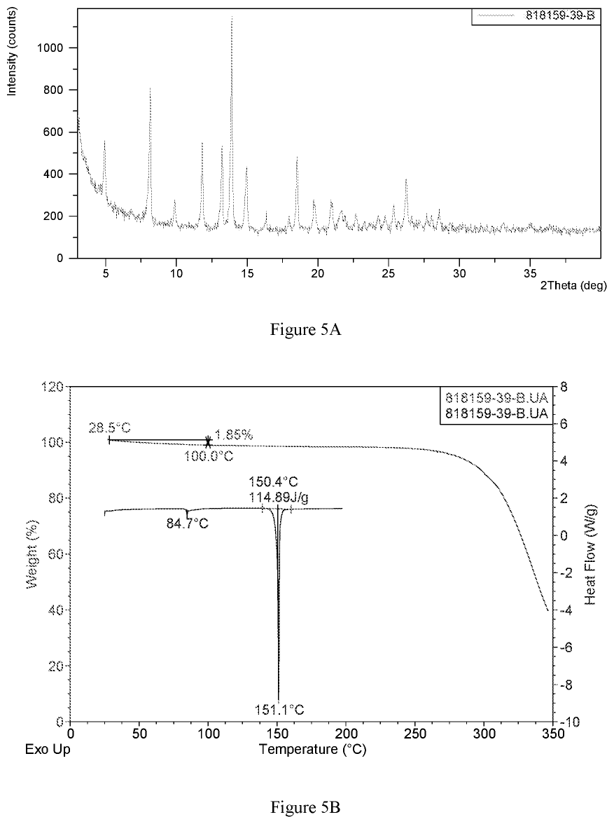

In some embodiments, the crystalline form B is characterized by an XRPD pattern comprising one or more peaks as shown in FIG. 5A. In some embodiments, crystalline form B is characterized by an XRPD pattern substantially as shown in FIG. 5A.

In some embodiments, the crystalline form B is characterized by a thermogravimetric analysis (TGA) thermogram substantially as shown in FIG. 5B.

In some embodiments, the crystalline form B is characterized by a differential scanning calorimetry (DSC) thermogram having an endotherm with a peak temperature of about 151.1° C.

In some embodiments, the crystalline form B is characterized by a DSC thermogram substantially as shown in FIG. 5B.

In some embodiments, the crystalline form B is an anhydrate.

In some embodiments, the crystalline form B is in substantially pure form. In another embodiment, the crystalline form B has a purity of at least 50 wt %, at least 60 wt %, at least 70 wt %, at least 80 wt %, at least 85 wt %, at least 86 wt %, at least 87 wt %, at least 88 wt %, at least 89 wt %, at least 90 wt %, at least 91 wt %, at least 92 wt %, at least 93 wt %, at least 94 wt %, at least 95 wt %, at least 96 wt %, at least 97 wt %, at least 98 wt %, or at least 99 wt %.

### Crystalline Form C of Compound 2

In one aspect, the present disclosure relates to a crystalline form C of Compound 2 characterized by an XRPD pattern comprising one or more peaks at 5.06, 10.09 and 20.24 (±0.2° 2θ). In some embodiments, the XRPD pattern of crystalline form C further comprises one or more peaks at 15.15, 16.18 and 16.75 (±0.2° 2θ). In some embodiments, the XRPD pattern of crystalline form C further comprises one or more peaks at 7.99, 8.37, 11.71, 12.22, 12.87, 13.60 and 14.04 (±0.2° 2θ).

In some embodiments, the crystalline form C is characterized by an XRPD pattern comprising one or more peaks selected from the group consisting of:

| °2θ | °2θ | °2θ | °2θ | |
|---|---|---|---|---|
| 5.06 | 12.22 | 16.18 | 25.89 | |
| 7.99 | 12.87 | 16.75 | 26.47 | |
| 8.37 | 13.60 | 19.43 | 30.56 | |
| 10.09 | 14.04 | 20.24 | 35.80 | |
| 11.71 | 15.15 | 20.96 | | . |

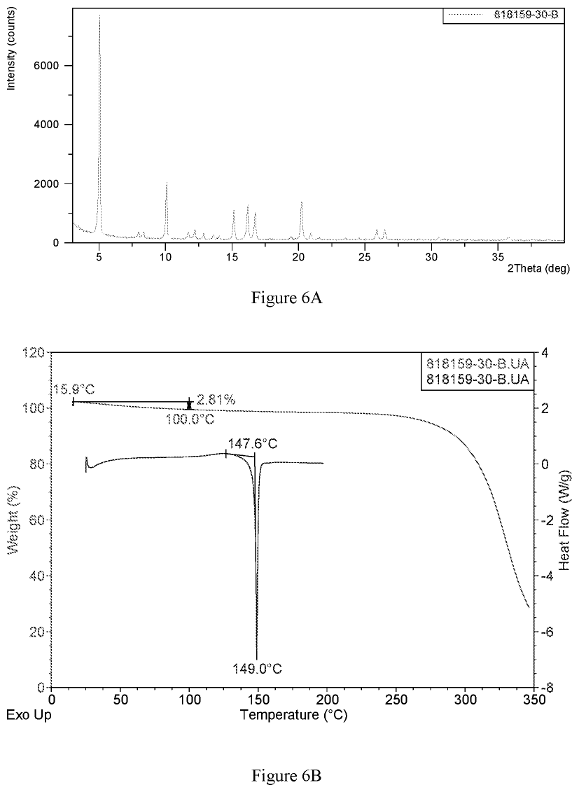

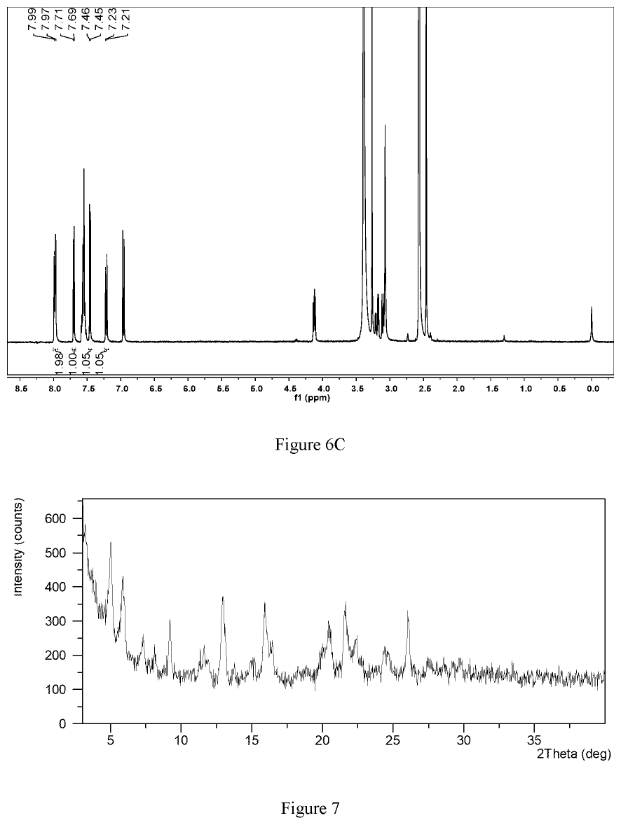

In some embodiments, the crystalline form C is characterized by an XRPD pattern comprising one or more peaks as shown in FIG. 6A. In some embodiments, crystalline form C is characterized by an XRPD pattern substantially as shown in FIG. 6A.

In some embodiments, the crystalline form C is characterized by a TGA thermogram substantially as shown in FIG. 6B.

In some embodiments, the crystalline form C is characterized by a DSC thermogram having an endotherm with a peak temperature of about 149.0° C.

In some embodiments, the crystalline form C is characterized by a DSC thermogram substantially as shown in FIG. 6B.

In some embodiments, the crystalline form C is an anhydrate.

In some embodiments, the crystalline form C is in substantially pure form. In another embodiment, the crystalline form C has a purity of at least 50 wt %, at least 60 wt %, at least 70 wt %, at least 80 wt %, at least 85 wt %, at least 86 wt %, at least 87 wt %, at least 88 wt %, at least 89 wt %, at least 90 wt %, at least 91 wt %, at least 92 wt %, at least 93 wt %, at least 94 wt %, at least 95 wt %, at least 96 wt %, at least 97 wt %, at least 98 wt %, or at least 99 wt %.

### Crystalline Form D of Compound 2

In one aspect, the present disclosure relates to a crystalline form D of Compound 2, characterized by an XRPD pattern comprising one or more peaks at 5.03, 12.98, 15.91 and 21.58 (±0.2° 2θ). In some embodiments, the XRPD pattern of crystalline form D further comprises one or more peaks at 5.90, 9.21, 20.47 and 26.08 (±0.2° 2θ). In some embodiments, the XRPD pattern of crystalline form D further comprises one or more peaks at 11.76, 15.04, 22.38 and 24.47 (±0.2° 2θ).

In some embodiments, the crystalline form D is characterized by an XRPD pattern comprising one or more peaks selected from the group consisting of:

| °2θ | °2θ | °2θ | °2θ | |
|---|---|---|---|---|
| 5.03 | 11.76 | 15.91 | 22.38 | |
| 5.90 | 12.98 | 20.47 | 24.47 | |
| 9.21 | 15.04 | 21.58 | 26.08 | . |

In some embodiments, the crystalline form D is characterized by an XRPD pattern comprising one or more peaks as shown in FIG. 7. In some embodiments, crystalline form D is characterized by an XRPD pattern substantially as shown in FIG. 7.

In some embodiments, the crystalline form D is a hydrate.

In some embodiments, the crystalline form D is in substantially pure form. In another embodiment, the crystalline form D has a purity of at least 50 wt %, at least 60 wt %, at least 70 wt %, at least 80 wt %, at least 85 wt %, at least 86 wt %, at least 87 wt %, at least 88 wt %, at least 89 wt %, at least 90 wt %, at least 91 wt %, at least 92 wt %, at least 93 wt %, at least 94 wt %, at least 95 wt %, at least 96 wt %, at least 97 wt %, at least 98 wt %, or at least 99 wt %.

### Crystalline Form E of Compound 2

In one aspect, the present disclosure relates to a crystalline form E of Compound 2 characterized by an XRPD pattern comprising one or more peaks at 10.12, 11.97, 13.73 and 18.89 (±0.2° 2θ). In some embodiments, the XRPD pattern of crystalline form E further comprises one or more peaks at 5.06, 8.14, 13.02 and 14.19 (±0.2° 2θ). In some embodiments, the XRPD pattern of crystalline form E further comprises one or more peaks at 14.87, 21.30, 22.33 and 26.23 (±0.2° 2θ).

In some embodiments, the crystalline form E is characterized by an XRPD pattern comprising one or more peaks selected from the group consisting of:

| °2θ | °2θ | °2θ | °2θ | |
|---|---|---|---|---|
| 5.06 | 16.32 | 22.33 | 27.18 | |
| 8.14 | 18.00 | 23.01 | 27.68 | |
| 10.12 | 18.89 | 23.78 | 28.57 | |
| 11.97 | 19.36 | 24.23 | 29.51 | |
| 13.02 | 19.89 | 25.07 | 31.40 | |
| 13.73 | 20.31 | 25.40 | 32.47 | |
| 14.19 | 20.67 | 26.23 | 36.45 | |
| 14.87 | 21.30 | 26.74 | 39.05 | . |

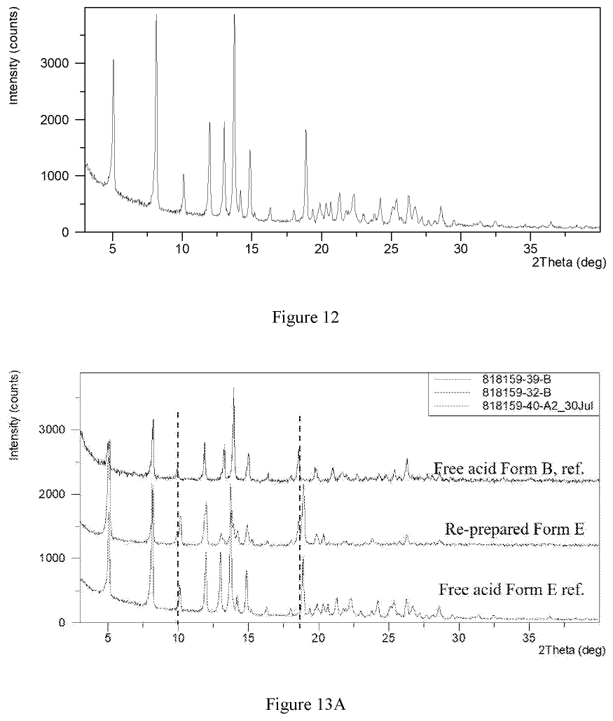

In some embodiments, the crystalline form E is characterized by an XRPD pattern comprising one or more peaks as shown in FIG. 12. In some embodiments, crystalline form E is characterized by an XRPD pattern substantially as shown in FIG. 12.

In some embodiments, the crystalline form E is an anhydrate.

In some embodiments, the crystalline form E is in substantially pure form. In some embodiments, the crystalline form E has a purity of at least 50 wt %, at least 60 wt %, at least 70 wt %, at least 80 wt %, at least 85 wt %, at least 86 wt %, at least 87 wt %, at least 88 wt %, at least 89 wt %, at least 90 wt %, at least 91 wt %, at least 92 wt %, at least 93 wt %, at least 94 wt %, at least 95 wt %, at least 96 wt %, at least 97 wt %, at least 98 wt %, or at least 99 wt %.

### Crystalline Form F of Compound 2

In one aspect, the present disclosure relates to a crystalline form F of Compound 2, characterized by an XRPD pattern comprising one or two peaks at 15.50 and 21.23 (±0.2° 2θ). In some embodiments, the XRPD pattern of crystalline form F further comprises one or two peaks at 12.12 and 20.21 (±0.2° 2θ).

In some embodiments, the crystalline form F is characterized by an XRPD pattern comprising one or more peaks selected from the group consisting of:

| °2θ | °2θ | °2θ | °2θ | |
|---|---|---|---|---|
| 12.12 | 15.50 | 20.21 | 21.23 | . |

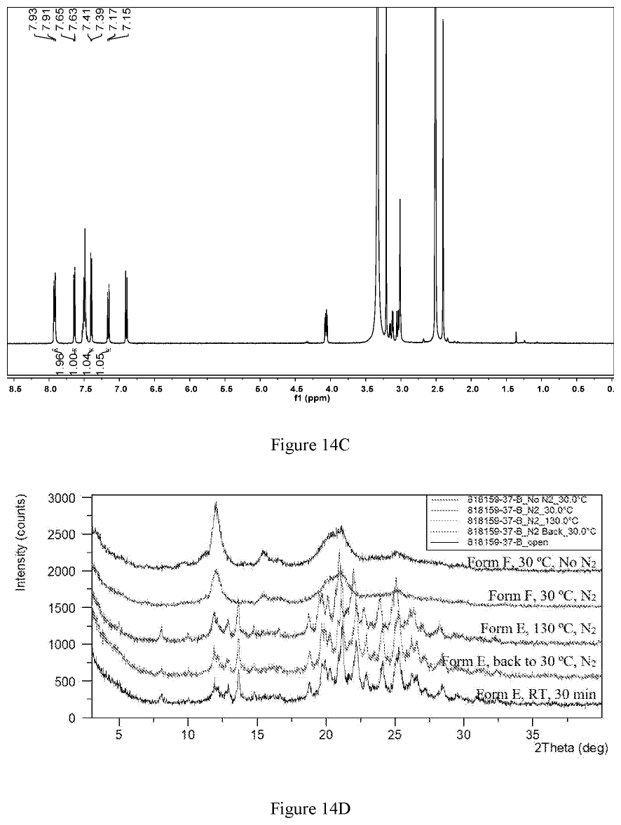

In some embodiments, the crystalline form F is characterized by an XRPD pattern comprising one or more peaks as shown in FIG. 14A. In some embodiments, crystalline form F is characterized by an XRPD pattern substantially as shown in FIG. 14A.

In some embodiments, the crystalline form F is characterized by a TGA thermogram substantially as shown in FIG. 14B.

In some embodiments, the crystalline form F is characterized by a differential scanning calorimetry (DSC) thermogram having endotherms with peak temperatures of about 111.2° C. and/or about 149.8° C.

In some embodiments, the crystalline form F is characterized by a DSC thermogram having an exothermal with a peak temperature of about 118.9° C.

In some embodiments, the crystalline form F is characterized by a DSC thermogram substantially as shown in FIG. 14B.

In some embodiments, the crystalline form F is a hydrate.

In some embodiments, the crystalline form F is in substantially pure form. In some embodiments, the crystalline form F has a purity of at least 50 wt %, at least 60 wt %, at least 70 wt %, at least 80 wt %, at least 85 wt %, at least 86 wt %, at least 87 wt %, at least 88 wt %, at least 89 wt %, at least 90 wt %, at least 91 wt %, at least 92 wt %, at least 93 wt %, at least 94 wt %, at least 95 wt %, at least 96 wt %, at least 97 wt %, at least 98 wt %, or at least 99 wt %.

### Crystalline Form G of Compound 2

In one aspect, the present disclosure relates to a crystalline form G of Compound 2, characterized by an XRPD pattern comprising one or more peaks at 9.67, 11.60, 12.90 and 14.43 (±0.2° 2θ). In some embodiments, the XRPD pattern of crystalline form G further comprises one or more peaks at 8.16, 13.69, 13.91, 16.15, 18.51, 18.93 and 20.47 (±0.2° 2θ). In some embodiments, the XRPD pattern of crystalline form G further comprises one or more peaks at 4.95, 11.83, 13.22 and 19.84 (±0.2° 2θ).

In some embodiments, the crystalline form G is characterized by an XRPD pattern comprising one or more peaks selected from the group consisting of:

| °2θ | °2θ | °2θ | °2θ | |
|---|---|---|---|---|
| 4.95 | 13.69 | 19.84 | 28.60 | |
| 8.16 | 13.91 | 20.47 | 29.94 | |
| 9.67 | 14.43 | 22.70 | 35.09 | |
| 11.60 | 14.84 | 25.28 | 35.93 | |
| 11.83 | 16.15 | 26.24 | | |
| 12.90 | 18.51 | 26.81 | | |
| 13.22 | 18.93 | 27.59 | | . |

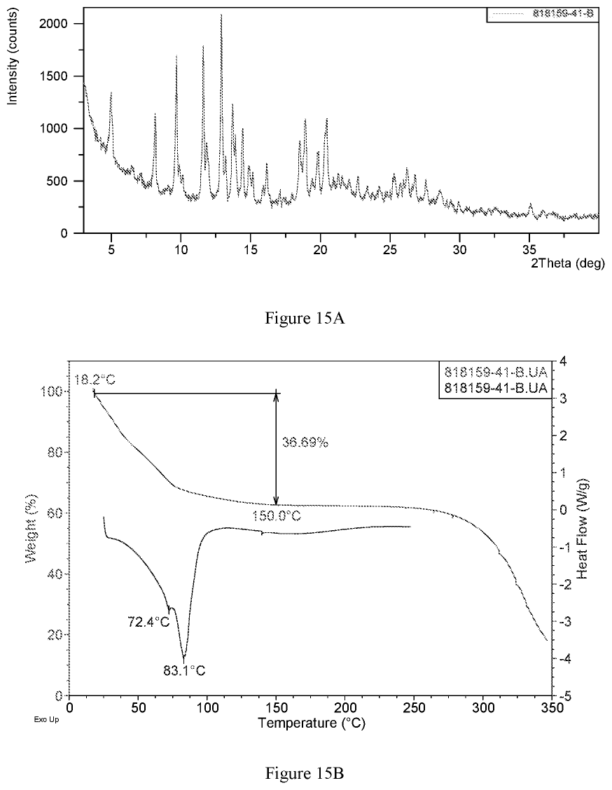

In some embodiments, the crystalline form G is characterized by an XRPD pattern comprising one or more peaks as shown in FIG. 15A. In some embodiments, crystalline form G is characterized by an XRPD pattern substantially as shown in FIG. 15A.

In some embodiments, the crystalline form G is characterized by a TGA thermogram substantially as shown in FIG. 15B.

In some embodiments, the crystalline form G is characterized by a DSC thermogram having endotherms with peak temperatures of about 72.4° C. and/or about 83.1° C.

In some embodiments, the crystalline form G is characterized by a DSC thermogram substantially as shown in FIG. 15B.

In some embodiments, the crystalline form G is a DMSO solvate.

In some embodiments, the crystalline form G is in substantially pure form. In some embodiments, the crystalline form G has a purity of at least 50 wt %, at least 60 wt %, at least 70 wt %, at least 80 wt %, at least 85 wt %, at least 86 wt %, at least 87 wt %, at least 88 wt %, at least 89 wt %, at least 90 wt %, at least 91 wt %, at least 92 wt %, at least 93 wt %, at least 94 wt %, at least 95 wt %, at least 96 wt %, at least 97 wt %, at least 98 wt %, or at least 99 wt %.

### Salt of Compound 2

In another aspect, the present disclosure provides a salt of Compound 2, wherein the salt of Compound 2 is selected from sodium salt, potassium salt, arginine salt, magnesium salt or tromethamine salt of compound 2.

### Crystalline Form A of Sodium Salt of Compound 2

In one aspect, the present disclosure relates to a crystalline form A of the sodium salt of Compound 2, characterized by an XRPD pattern comprising one or more peaks at 4.09, 4.62 and 14.21 (±0.2° 2θ). In some embodiments, the XRPD pattern of crystalline form A of sodium salt further comprises one or two peaks at 16.43 and 17.72 (±0.2° 2θ).

In some embodiments, the crystalline form A of sodium salt is characterized by an XRPD pattern comprising one or more peaks selected from the group consisting of:

| °2θ | °2θ | °2θ | |
|---|---|---|---|
| 4.09 | 14.21 | 17.72 | |
| 4.62 | 16.43 | | . |

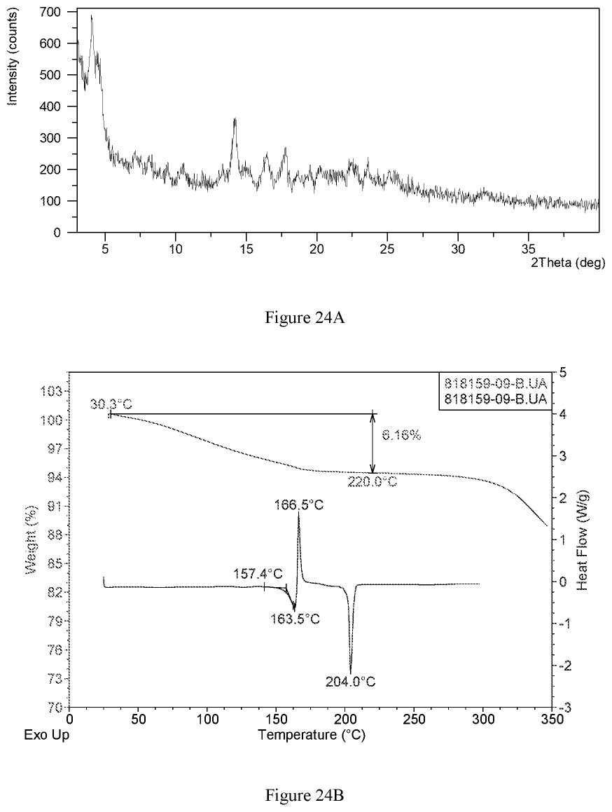

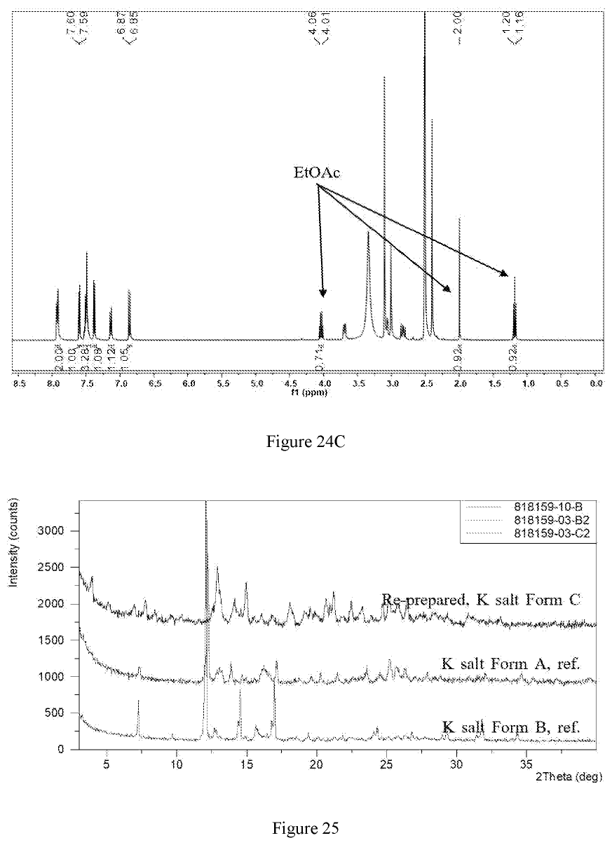

In some embodiments, the crystalline form A of sodium salt is characterized by an XRPD pattern comprising one or more peaks as shown in FIG. 24A. In some embodiments, crystalline form A of sodium salt is characterized by an XRPD pattern substantially as shown in FIG. 24A.

In some embodiments, the crystalline form A of sodium salt is characterized by a TGA thermogram substantially as shown in FIG. 24B.

In some embodiments, the crystalline form A of sodium salt is characterized by a DSC thermogram having endotherms with peak temperatures of about 163.5° C. and/or about 204.0° C.

In some embodiments, the crystalline form A of sodium salt is characterized by a DSC thermogram having an exothermal with a peak temperature of about 166.5° C.

In some embodiments, the crystalline form A of sodium salt is characterized by a DSC thermogram substantially as shown in FIG. 24B.

In some embodiments, the crystalline form A of sodium salt is in substantially pure form. In some embodiments, the crystalline form A of sodium salt has a purity of at least 50 wt %, at least 60 wt %, at least 70 wt %, at least 80 wt %, at least 85 wt %, at least 86 wt %, at least 87 wt %, at least 88 wt %, at least 89 wt %, at least 90 wt %, at least 91 wt %, at least 92 wt %, at least 93 wt %, at least 94 wt %, at least 95 wt %, at least 96 wt %, at least 97 wt %, at least 98 wt %, or at least 99 wt %.

### Crystalline Form C of Potassium Salt of Compound 2

In one aspect, the present disclosure relates to a crystalline form C of potassium salt of Compound 2, characterized by an XRPD pattern comprising one or more peaks at 12.91, 14.96 and 21.23 (±0.2° 2θ). In some embodiments, the XRPD pattern of crystalline form C of potassium salt further comprises one or more peaks at 14.12, 20.68, 25.17 and 26.46 (±0.2° 2θ). In some embodiments, the XRPD pattern of crystalline form C of potassium salt further comprises one or more peaks at 3.92, 18.08, 22.48, 24.70 and 25.81 (±0.2° 2θ).

In some embodiments, the crystalline form C of potassium salt is characterized by an XRPD pattern comprising one or more peaks selected from the group consisting of:

| °2θ | °2θ | °2θ | °2θ | |
|---|---|---|---|---|
| 3.92 | 14.96 | 20.68 | 25.17 | |
| 6.90 | 16.05 | 21.23 | 25.81 | |
| 7.79 | 16.83 | 22.48 | 26.46 | |
| 12.91 | 18.08 | 23.28 | 30.84 | |
| 14.12 | 19.11 | 24.70 | | . |

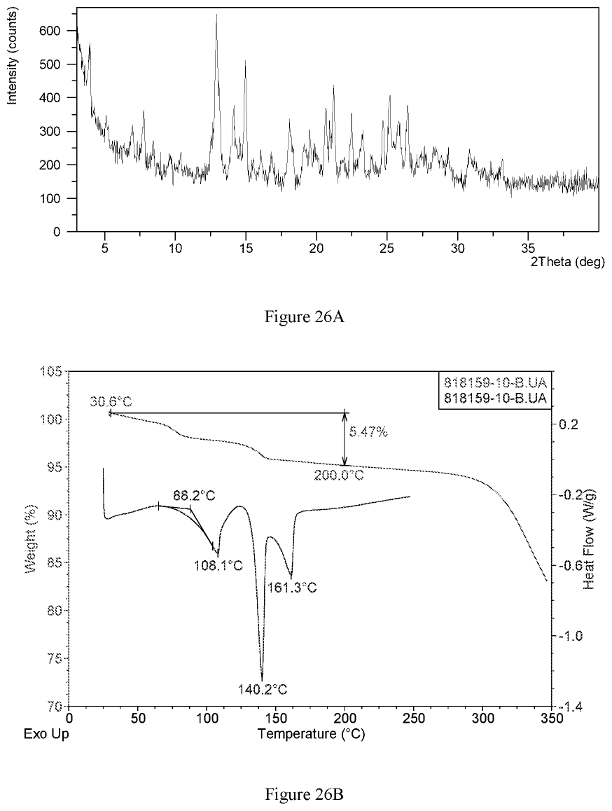

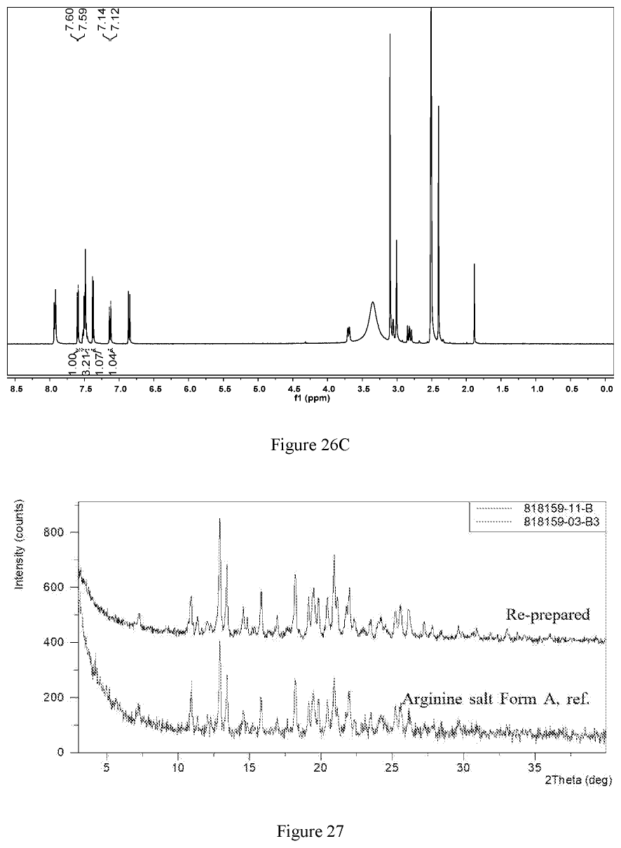

In some embodiments, the crystalline form C of potassium salt is characterized by an XRPD pattern comprising one or more peaks as shown in FIG. 26A. In some embodiments, crystalline form C of potassium salt is characterized by an XRPD pattern substantially as shown in FIG. 26A.

In some embodiments, the crystalline form C of potassium salt is characterized by a TGA thermogram substantially as shown in FIG. 26B.

In some embodiments, the crystalline form C of potassium salt is characterized by a DSC thermogram having endotherms with peak temperatures of about 108.1° C., about 140.2° C. and/or about 161.3° C.

In some embodiments, the crystalline form C of potassium salt is characterized by a DSC thermogram substantially as shown in FIG. 26B.

In some embodiments, the crystalline form C of potassium salt is in substantially pure form. In some embodiments, the crystalline form C of potassium salt has a purity of at least 50 wt %, at least 60 wt %, at least 70 wt %, at least 80 wt %, at least 85 wt %, at least 86 wt %, at least 87 wt %, at least 88 wt %, at least 89 wt %, at least 90 wt %, at least 91 wt %, at least 92 wt %, at least 93 wt %, at least 94 wt %, at least 95 wt %, at least 96 wt %, at least 97 wt %, at least 98 wt %, or at least 99 wt %.

### Crystalline Form A of Arginine Salt of Compound 2

In one aspect, the present disclosure relates to a crystalline form A of arginine salt of Compound 2, characterized by an XRPD pattern comprising one or more peaks at 12.93, 13.43, 18.21 and 19.49 (±0.2° 2θ). In some embodiments, the XRPD pattern of crystalline form A of arginine salt further comprises one or more peaks at 10.91, 15.83, 19.14, 20.93, 21.15 and 22.02 (±0.2° 2θ). In some embodiments, the XRPD pattern of crystalline form A of arginine salt further comprises one or more peaks at 11.34, 19.84, 20.45, 25.63 and 26.15 (±0.2° 2θ).

In some embodiments, the crystalline form A of arginine salt is characterized by an XRPD pattern comprising one or more peaks selected from the group consisting of:

| °2θ | °2θ | °2θ | °2θ | |
|---|---|---|---|---|
| 7.25 | 16.94 | 21.15 | 27.24 | |
| 10.91 | 18.21 | 22.02 | 27.81 | |
| 11.34 | 19.14 | 23.46 | 29.67 | |
| 12.93 | 19.49 | 24.10 | 30.90 | |
| 13.43 | 19.84 | 25.22 | 33.07 | |
| 14.57 | 20.45 | 25.63 | | |
| 15.83 | 20.93 | 26.15 | | . |

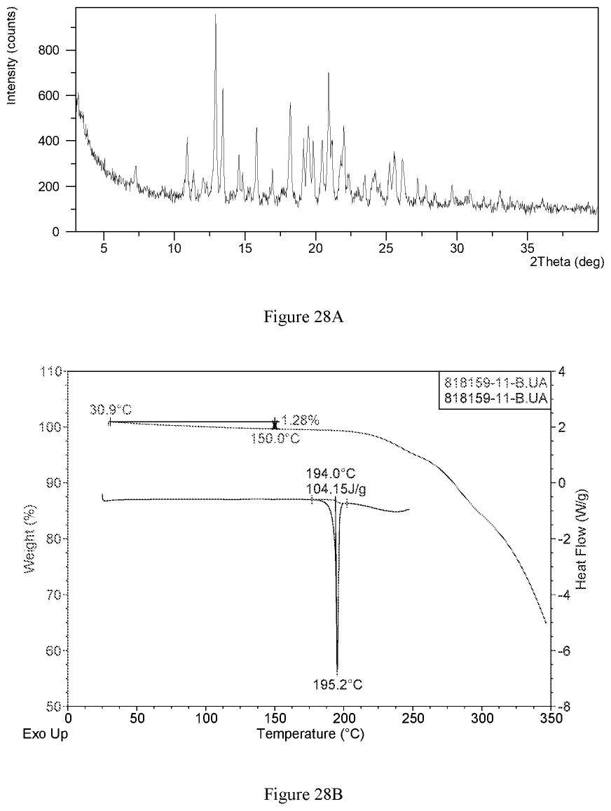

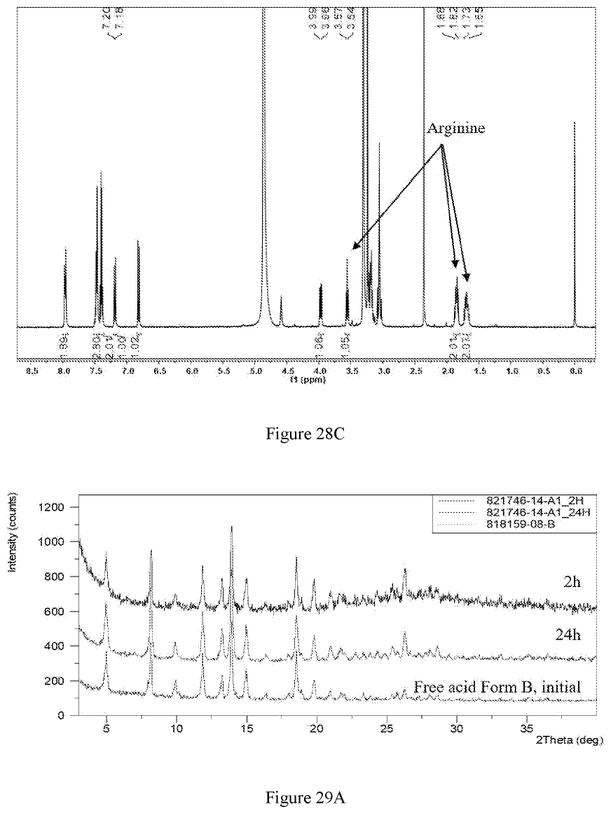

In some embodiments, the crystalline form A of arginine salt is characterized by an XRPD pattern comprising one or more peaks as shown in FIG. 28A. In some embodiments, crystalline form A of arginine salt is characterized by an XRPD pattern substantially as shown in FIG. 28A.

In some embodiments, the crystalline form A of arginine salt is characterized by a TGA thermogram substantially as shown in FIG. 28B.

In some embodiments, the crystalline form A of arginine salt is characterized by a DSC thermogram having an endotherm with a peak temperature of about 195.2° C.

In some embodiments, the crystalline form A of arginine salt is characterized by a DSC thermogram substantially as shown in FIG. 28B.

In some embodiments, the crystalline form A of arginine salt is in substantially pure form. In some embodiments, the crystalline form A of arginine salt has a purity of at least 50 wt %, at least 60 wt %, at least 70 wt %, at least 80 wt %, at least 85 wt %, at least 86 wt %, at least 87 wt %, at least 88 wt %, at least 89 wt %, at least 90 wt %, at least 91 wt %, at least 92 wt %, at least 93 wt %, at least 94 wt %, at least 95 wt %, at least 96 wt %, at least 97 wt %, at least 98 wt %, or at least 99 wt %.

### Crystalline Form A of Potassium Salt of Compound 2

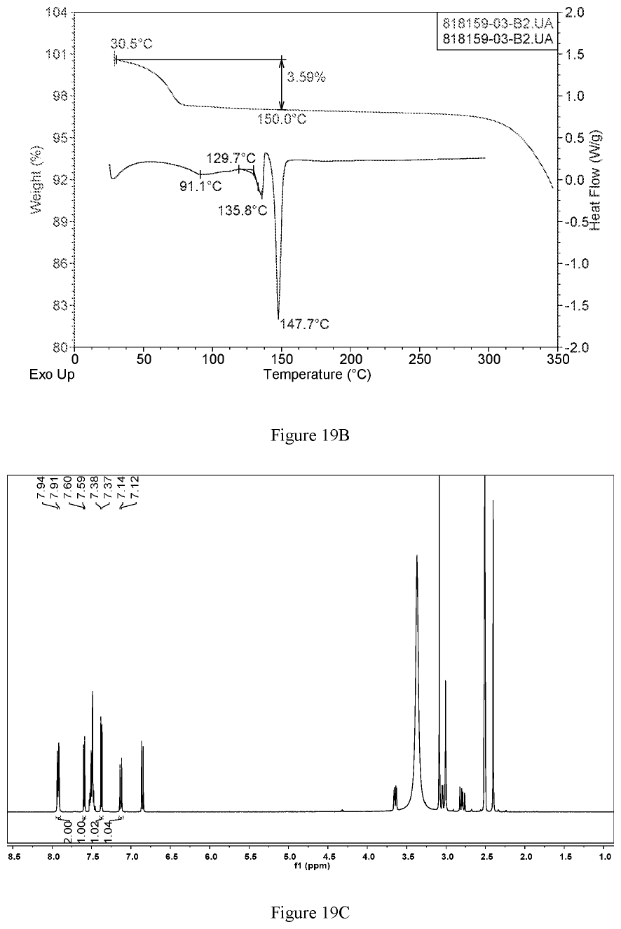

In one aspect, the present disclosure relates to a crystalline form A of potassium salt of Compound 2, characterized by an XRPD pattern comprising one or more peaks as shown in the upper curve of FIG. 19A. In some embodiments, crystalline form A of potassium salt is characterized by an XRPD pattern substantially as shown in the upper curve of FIG. 19A.

In some embodiments, the crystalline form A of potassium salt is characterized by a TGA thermogram substantially as shown in FIG. 19B.

In some embodiments, the crystalline form A of potassium salt is characterized by a DSC thermogram having endotherms with peak temperatures of about 91.1° C., about 135.8° C. and/or about 147.7° C.

In some embodiments, the crystalline form A of potassium salt is characterized by a DSC thermogram substantially as shown in FIG. 19B.

In some embodiments, the crystalline form A of potassium salt is in substantially pure form. In some embodiments, the crystalline form A of potassium salt has a purity of at least 50 wt %, at least 60 wt %, at least 70 wt %, at least 80 wt %, at least 85 wt %, at least 86 wt %, at least 87 wt %, at least 88 wt %, at least 89 wt %, at least 90 wt %, at least 91 wt %, at least 92 wt %, at least 93 wt %, at least 94 wt %, at least 95 wt %, at least 96 wt %, at least 97 wt %, at least 98 wt %, or at least 99 wt %.

### Crystalline Form B of Potassium Salt of Compound 2

In one aspect, the present disclosure relates to a crystalline form B of potassium salt of Compound 2, characterized by an XRPD pattern comprising one or more peaks as shown in the lower curve of FIG. 19A. In some embodiments, crystalline form B of potassium salt is characterized by an XRPD pattern substantially as shown in the lower curve of FIG. 19A.

In some embodiments, the crystalline form B of potassium salt is in substantially pure form. In some embodiments, the crystalline form B of potassium salt has a purity of at least 50 wt %, at least 60 wt %, at least 70 wt %, at least 80 wt %, at least 85 wt %, at least 86 wt %, at least 87 wt %, at least 88 wt %, at least 89 wt %, at least 90 wt %, at least 91 wt %, at least 92 wt %, at least 93 wt %, at least 94 wt %, at least 95 wt %, at least 96 wt %, at least 97 wt %, at least 98 wt %, or at least 99 wt %.

### Crystalline Form A of Magnesium Salt of Compound 2

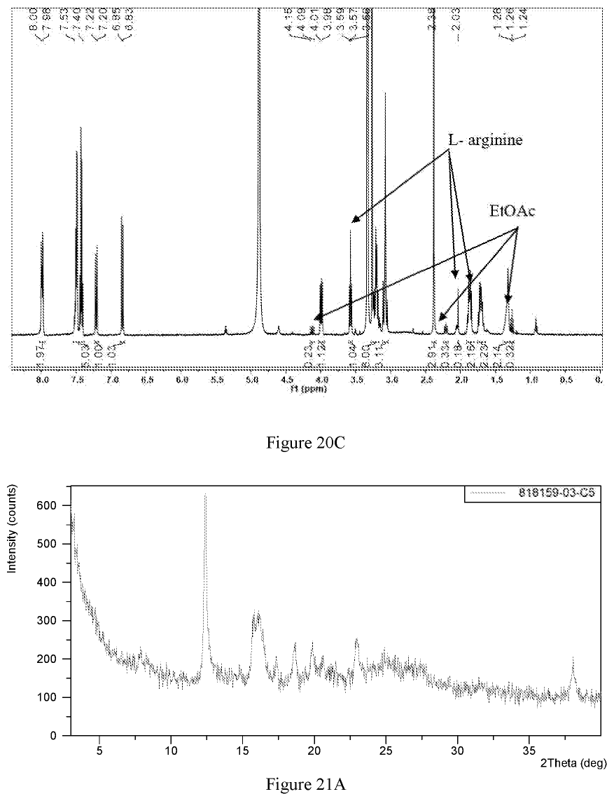

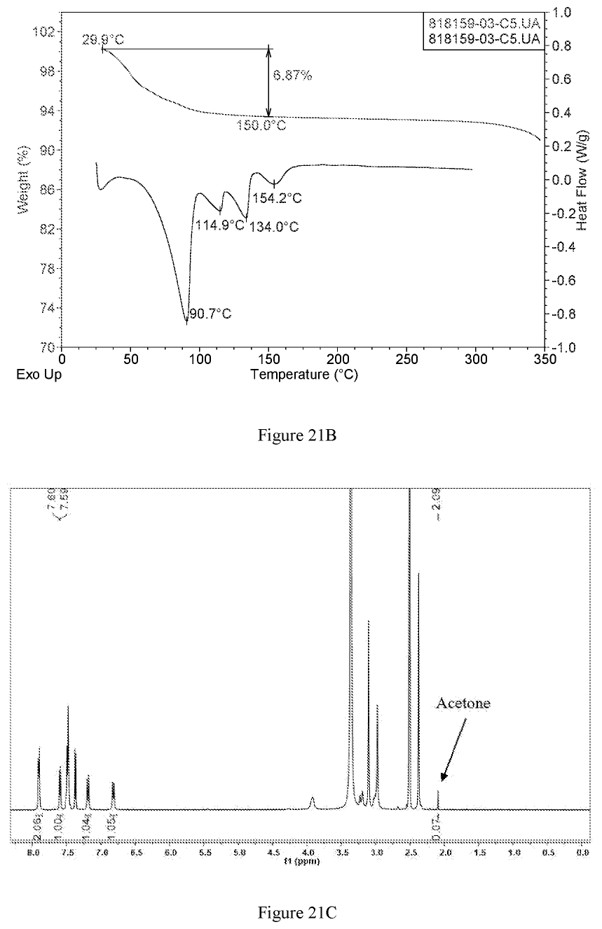

In one aspect, the present disclosure relates to a crystalline form A of magnesium salt of Compound 2, characterized by an XRPD pattern comprising one or more peaks as shown in FIG. 21A. In some embodiments, crystalline form A of magnesium salt is characterized by an XRPD pattern substantially as shown in FIG. 21A.

In some embodiments, the crystalline form A of magnesium salt is characterized by a TGA thermogram substantially as shown in FIG. 21B.

In some embodiments, the crystalline form A of magnesium salt is characterized by a DSC thermogram having endotherms with peak temperatures of about 90.7° C., about 114.9° C., about 134.0° C. and/or about 154.2° C.

In some embodiments, the crystalline form A of magnesium salt is characterized by a DSC thermogram substantially as shown in FIG. 21B.

In some embodiments, the crystalline form A of magnesium salt is in substantially pure form. In some embodiments, the crystalline form A of magnesium salt has a purity of at least 50 wt %, at least 60 wt %, at least 70 wt %, at least 80 wt %, at least 85 wt %, at least 86 wt %, at least 87 wt %, at least 88 wt %, at least 89 wt %, at least 90 wt %, at least 91 wt %, at least 92 wt %, at least 93 wt %, at least 94 wt %, at least 95 wt %, at least 96 wt %, at least 97 wt %, at least 98 wt %, or at least 99 wt %.

### Crystalline Form A of Tromethamine Salt of Compound 2

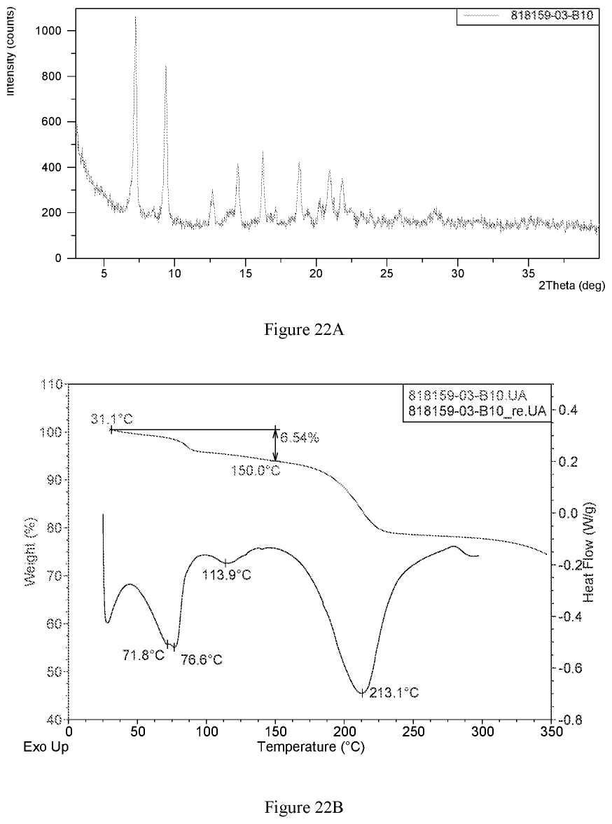

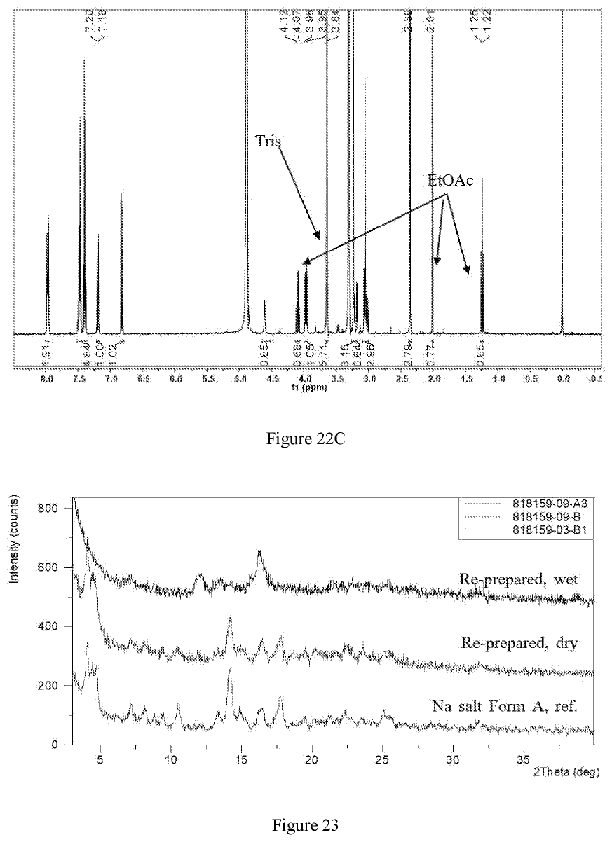

In one aspect, the present disclosure relates to a crystalline form A of tromethamine salt of Compound 2, characterized by an XRPD pattern comprising one or more peaks as shown in FIG. 22A. In some embodiments, crystalline form A of tromethamine salt is characterized by an XRPD pattern substantially as shown in FIG. 22A.

In some embodiments, the crystalline form A of tromethamine salt is characterized by a TGA thermogram substantially as shown in FIG. 22B.

In some embodiments, the crystalline form A of tromethamine salt is characterized by a DSC thermogram having endotherms with peak temperatures of about 71.8° C., about 76.6° C., about 113.9° C. and/or about 213.1° C.

In some embodiments, the crystalline form A of tromethamine salt is characterized by a DSC thermogram substantially as shown in FIG. 22B.

In some embodiments, the crystalline form A of tromethamine salt is in substantially pure form. In some embodiments, the crystalline form A of tromethamine salt has a purity of at least 50 wt %, at least 60 wt %, at least 70 wt %, at least 80 wt %, at least 85 wt %, at least 86 wt %, at least 87 wt %, at least 88 wt %, at least 89 wt %, at least 90 wt %, at least 91 wt %, at least 92 wt %, at least 93 wt %, at least 94 wt %, at least 95 wt %, at least 96 wt %, at least 97 wt %, at least 98 wt %, or at least 99 wt %.

### Synthesis of Compound 2

Compound 2 can be synthesized according to methods known to persons skilled in the art, such as the synthetic procedures described in Example 1.

### Pharmaceutical Compositions

In a further aspect, there is provided pharmaceutical compositions comprising Compound 2 or a salt of Compound 2, wherein Compound 2 or the salt of Compound 2 is in a crystalline form selected from the group consisting of: the crystalline form B, the crystalline form C, the crystalline form D, the crystalline form E, the crystalline form F, the crystalline form G, the crystalline form A of sodium salt, the crystalline form C of potassium salt, the crystalline form A of arginine salt, the crystalline form A of potassium salt, the crystalline form B of potassium salt, the crystalline form A of magnesium salt, and the crystalline form A of tromethamine salt of the present disclosure.

In another aspect, there is provided pharmaceutical compositions comprising Compound 2 or a salt of Compound 2, wherein Compound 2 or the salt of Compound 2 is in a crystalline form selected from the group consisting of: the crystalline form B, the crystalline form C, the crystalline form D, the crystalline form E, the crystalline form F, the crystalline form G, the crystalline form A of sodium salt, the crystalline form C of potassium salt, the crystalline form A of arginine salt, the crystalline form A of potassium salt, the crystalline form B of potassium salt, the crystalline form A of magnesium salt, and the crystalline form A of tromethamine salt of the present disclosure, and at least one pharmaceutical acceptable excipient.

In some embodiments, at least 50%, 60%, 70%, 80%, 85%, 90%, 95%, 99%, 99.5%, 99.9% or 99.99% of Compound 2 is in crystalline form B.

In some embodiments, at least 50%, 60%, 70%, 80%, 85%, 90%, 95%, 99%, 99.5%, 99.9% or 99.99% of Compound 2 is in crystalline form C.

In some embodiments, at least 50%, 60%, 70%, 80%, 85%, 90%, 95%, 99%, 99.5%, 99.9% or 99.99% of Compound 2 is in crystalline form D.

In some embodiments, at least 50%, 60%, 70%, 80%, 85%, 90%, 95%, 99%, 99.5%, 99.9% or 99.99% of Compound 2 is in crystalline form E.

In some embodiments, at least 50%, 60%, 70%, 80%, 85%, 90%, 95%, 99%, 99.5%, 99.9% or 99.99% of Compound 2 is in crystalline form F.

In some embodiments, at least 50%, 60%, 70%, 80%, 85%, 90%, 95%, 99%, 99.5%, 99.9% or 99.99% of the salt of Compound 2 is in crystalline form G.

In some embodiments, at least 50%, 60%, 70%, 80%, 85%, 90%, 95%, 99%, 99.5%, 99.9% or 99.99% of the salt of Compound 2 is in the crystalline form A of sodium salt.

In some embodiments, at least 50%, 60%, 70%, 80%, 85%, 90%, 95%, 99%, 99.5%, 99.9% or 99.99% of the salt of Compound 2 is in the crystalline form C of potassium salt.

In some embodiments, at least 50%, 60%, 70%, 80%, 85%, 90%, 95%, 99%, 99.5%, 99.9% or 99.99% of the salt of Compound 2 is in the crystalline form A of arginine salt.

In some embodiments, at least 50%, 60%, 70%, 80%, 85%, 90%, 95%, 99%, 99.5%, 99.9% or 99.99% of the salt of Compound 2 is in the crystalline form A of potassium salt.

In some embodiments, at least 50%, 60%, 70%, 80%, 85%, 90%, 95%, 99%, 99.5%, 99.9% or 99.99% of the salt of Compound 2 is in the crystalline form B of potassium salt.

In some embodiments, at least 50%, 60%, 70%, 80%, 85%, 90%, 95%, 99%, 99.5%, 99.9% or 99.99% of the salt of Compound 2 is in the crystalline form A of magnesium salt.

In some embodiments, at least 50%, 60%, 70%, 80%, 85%, 90%, 95%, 99%, 99.5%, 99.9% or 99.99% of the salt of Compound 2 is in the crystalline form A of tromethamine salt.

The particular excipient used in the pharmaceutical compositions provided herein will depend upon the means and purpose for which the compounds of the present disclosure is being applied. Solvents are generally selected based on solvents recognized by persons skilled in the art as safe to be administered to a mammal including humans. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof.

In some embodiments, suitable excipients may include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, suitable excipients may include one or more stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present disclosure or pharmaceutical composition thereof) or aid in the manufacturing of the pharmaceutical product (i.e., medicament). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16^{th} edition, Osol, A. Ed. (1980). A “liposome” is a small vesicle composed of various types of lipids, phospholipids and/or surfactant which is useful for delivery of a drug (such as the compounds disclosed herein and, optionally, a chemotherapeutic agent) to a mammal including humans. The components of the liposome are commonly arranged in a bilayer formation, similar to the lipid arrangement of biological membranes.

The pharmaceutical compositions provided herein can be in any form that allows for the composition to be administered to a subject, including, but not limited to a human, and formulated to be compatible with an intended route of administration.

A variety of routes are contemplated for the pharmaceutical compositions provided herein, and accordingly the pharmaceutical composition provided herein may be supplied in bulk or in unit dosage form depending on the intended administration route. For example, for oral, buccal, and sublingual administration, powders, suspensions, granules, tablets, pills, capsules, gelcaps, and caplets may be acceptable as solid dosage forms, and emulsions, syrups, elixirs, suspensions, and solutions may be acceptable as liquid dosage forms. For injection administration, emulsions and suspensions may be acceptable as liquid dosage forms, and a powder suitable for reconstitution with an appropriate solution as solid dosage forms. For inhalation administration, solutions, sprays, dry powders, and aerosols may be acceptable dosage form. For topical (including buccal and sublingual) or transdermal administration, powders, sprays, ointments, pastes, creams, lotions, gels, solutions, and patches may be acceptable dosage form. For vaginal administration, pessaries, tampons, creams, gels, pastes, foams and spray may be acceptable dosage form.

The quantity of active ingredient in a unit dosage form of composition is a therapeutically effective amount and is varied according to the particular treatment involved. As used herein, the term “therapeutically effective amount” refers to an amount of a molecule, compound, or composition comprising the molecule or compound to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; the rate of administration; the therapeutic or combination of therapeutics selected for administration; and the discretion of the prescribing physician. Therapeutically effective amounts for a given situation can be determined by routine experimentation that is within the skill and judgment of the clinician.

In some embodiments, an effective amount of the compound provide herein can range from about 0.5 μg per day to about 90 mg per day, 1 μg per day to about 50 mg per day, 2 μg per day to about 10 mg per day, 3 μg per day to about 1 mg per day, 5 μg per day to about 800 μg per day, 5 μg per day to about 600 μg per day, 5 μg per day to about 500 μg per day, 10 μg per day to about 500 μg per day, 12 μg per day to about 500 μg per day, 15 μg per day to about 500 μg per day, 20 μg per day to about 500 μg per day, 25 μg per day to about 500 μg per day. In some embodiments, an effective amount of the compound provide herein can range from about 25 μg per day to about 300 μg per day. In some embodiments, an effective amount of the compound provide herein can range from about 50 μg per day to about 150 μg per day.

In some embodiments, the pharmaceutical compositions of the present disclosure may be in a form of formulation for oral administration.

In certain embodiments, the pharmaceutical compositions of the present disclosure may be in the form of tablet formulations. Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

In certain embodiments, the pharmaceutical compositions of the present disclosure may be in a form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

In certain embodiments, the pharmaceutical compositions of the present disclosure may be in the form of aqueous suspensions, which generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

In certain embodiments, the pharmaceutical compositions of the present disclosure may be in the form of oily suspensions, which generally contain suspended active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

In certain embodiments, the pharmaceutical compositions of the present disclosure may be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

In certain embodiments, the pharmaceutical compositions provided herein may be in the form of syrups and elixirs, which may contain sweetening agents such as glycerol, propylene glycol, sorbitol, aspartame or sucrose, a demulcent, a preservative, a flavoring and/or coloring agent.

In some embodiments, the pharmaceutical compositions of the present disclosure may be in a form of formulation for injection administration.

In certain embodiments, the pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

In some embodiments, the pharmaceutical compositions of the present disclosure may be in a form of formulation for inhalation administration.

In certain embodiments, the pharmaceutical compositions of the present disclosure may be in the form of aqueous and nonaqueous (e.g., in a fluorocarbon propellant) aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols.

In some embodiments, the pharmaceutical compositions of the present disclosure may be in a form of formulation for topical or transdermal administration.

In certain embodiments, the pharmaceutical compositions provided herein may be in the form of creams, ointments, gels and aqueous or oily solutions or suspensions, which may generally be obtained by formulating an active ingredient with a conventional, topically acceptable excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In certain embodiments, the pharmaceutical compositions provided herein may be formulated in the form of transdermal skin patches that are well known to those of ordinary skill in the art.

Besides those representative dosage forms described above, pharmaceutically acceptable excipients and carriers are generally known to those skilled in the art and are thus included in the present disclosure. Such excipients and carriers are described, for example, in “Remingtons Pharmaceutical Sciences” Mack Pub. Co., New Jersey (1991), in “Remington: The Science and Practice of Pharmacy”, Ed. University of the Sciences in Philadelphia, 21^{st} Edition, LWW (2005), which are incorporated herein by reference.

In some embodiments, the pharmaceutical compositions of the present disclosure can be formulated as a single dosage form. The amount of the compounds provided herein in the single dosage form will vary depending on the subject treated and particular mode of administration.

In some embodiments, the pharmaceutical compositions of the present disclosure can be formulated as short-acting, fast-releasing, long-acting, and sustained-releasing. Accordingly, the pharmaceutical formulations of the present disclosure may also be formulated for controlled release or for slow release.

In a further aspect, there is also provided veterinary compositions comprising one or more molecules or compounds of the present disclosure or pharmaceutically acceptable salts thereof and a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

The pharmaceutical compositions or veterinary compositions may be packaged in a variety of ways depending upon the method used for administering the drug. For example, an article for distribution can include a container having deposited therein the compositions in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings. The compositions may also be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described.

In a further aspect, there is also provided pharmaceutical compositions comprise one or more compounds of the present disclosure, or a pharmaceutically acceptable salt thereof, as a first active ingredient, and a second active ingredient.

For pharmaceutical compositions that comprise a second active agent, an effective amount of the second active agent is between about 20% and 100% of the dosage normally utilized in a monotherapy regime using just that agent. Preferably, an effective amount is between about 70% and 100% of the normal monotherapeutic dose. The normal monotherapeutic dosages of these second active agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second active agents will act synergistically with the compound provided herein. When this occurs, it will allow the effective dosage of the second active agent and/or the compound provided herein to be reduced from that required in a monotherapy. This has the advantage of minimizing toxic side effects of either the second active agent or the compound provided herein, synergistic improvements in efficacy, improved ease of administration or use and/or reduced overall expense of compound preparation or formulation.

In some embodiments, the second active agent can include: (1) a cholesterol absorption inhibitor, (2) an HMG-CoA reductase inhibitor, (3) a bile acid sequestrant, (4) nicotinyl alcohol, nicotinic acid or a salt thereof, (5) a phenolic anti-oxidant, (6) an ACAT inhibitor, and (7) a CTEP inhibitor.

### Method of Treatment of Disease

In another aspect, the present disclosure provides the crystalline form of Compound 2 or the salt of Compound 2 of the present disclosure or the pharmaceutical composition of the present disclosure, for use in a method for the treatment and/or prophylaxis of diseases which are modulated by PPARα and/or PPARγ agonists.

In another aspect, the present disclosure provides a method for the treatment and/or prophylaxis of diseases which are modulated by PPARα and/or PPARγ agonists in a subject, comprising administering an effective amount of the crystalline form of Compound 2 or the salt of Compound 2 of the present disclosure or the pharmaceutical composition of the present disclosure.

In another aspect, the present disclosure provides the use of the crystalline form of Compound 2 or the salt of Compound 2 of the present disclosure or the pharmaceutical composition of the present disclosure in the manufacture of a medicament for the treatment and/or prophylaxis of diseases which are modulated by PPARα and/or PPARγ agonists.

As used herein, the terms “treat”, “treating” and “treatment” are meant to include improving, preventing, alleviating or abrogating a disorder; or alleviating, preventing or abrogating one or more of the symptoms associated with the disorder; and/or preventing, alleviating or eradicating the cause(s) of the disorder itself, i.e., causing a clinical symptom to not significantly develop in a mammal that may be predisposed to the disease but does not yet experience or display symptoms of the disease. This may include improving the subject's ability to perform activities of daily living, perform domestic chores, manage finances, and/or perform an occupation or reduce the level of care needed by the subject. Treat, treating or treatment may include improvement of the symptom by at least 20%, 30%, 50%, 80%, 90%, or by 100%. Symptoms associated with a specific disorder depend on the specific disorder at hand.

As used herein, the term “prophylaxis” or “prophylactic” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

As used herein, the term “disease” means any condition or disorder that damages or interferes with the normal function of a cell, tissue, or organ.

In some embodiments, the disease is diabetes, non-insulin dependent diabetes mellitus, elevated blood pressure, dyslipidemia, atherosclerotic diseases, metabolic syndrome, or diabetic nephropathy.

In some embodiments, the disease is non-insulin dependent diabetes mellitus, or diabetic nephropathy.

In some embodiments, the disease is diabetic nephropathy.

As used herein, the term “diabetes” means a disease in which a patient's ability to control glucose levels in blood is impaired, because of the partially lost the ability to respond properly to the action of insulin.

As used herein, the term “non-insulin dependent diabetes mellitus” also referred to type II diabetes (T2D), which afflicts 80-90% of all diabetic patients in developed countries, the Isles of Langerhans in the pancreas still produce insulin. However, the target organs, mainly muscle, liver and adipose tissue, exhibit a profound resistance to insulin stimulation, and the body compensates by producing unphysiologically high levels of insulin. In later stage of disease, however, insulin secretion decreases due to exhaustion of the pancreas.

As used herein, the term “atherosclerotic disease”, also known as arteriosclerotic vascular disease or ASVD, is a specific form of arteriosclerosis in which an artery wall thickens as a result of invasion and accumulation of white blood cells (foam cells) and proliferation of intimal-smooth-muscle cell creating an atheromatous (fibrofatty) plaque.

As used herein, the term “metabolic syndrome” means a cluster of conditions that occur together, increasing the risk of heart disease, stroke and type 2 diabetes. These conditions include increased blood pressure, high blood sugar, excess body fat around the waist, and abnormal cholesterol or triglyceride levels.

As used herein, the term “diabetic nephropathy” means a kidney disease that results from diabetes, which is the number one cause of kidney failure. Almost a third of people with diabetes develop diabetic nephropathy. There are often no symptoms with early diabetic nephropathy. As the kidney function worsens, symptoms may include: swelling of the hands, feet, and face; trouble sleeping or concentrating; poor appetite; nausea; weakness; itching (end-stage kidney disease) and extremely dry skin; drowsiness (end-stage kidney disease); abnormalities in the hearts' regular rhythm, because of increased potassium in the blood; and muscle twitching.

In some embodiments, the disease is renal injury. In some embodiments, the renal injury is caused by ureteral obstruction. In some embodiments, the renal injury is caused by unilateral ureteral obstruction.

As used herein, the term “PPARα/γ dual agonist”, refers to compounds that exhibit both significant PPARα and PPARγ agonism. In some embodiments, PPARα/γ dual agonist exhibits significant PPARα and/or PPARγ agonism, wherein the half-maximal concentration potencies (EC₅₀) for activation of hPPARγ and EC₅₀ for activation of hPPARα differ by less than 30-fold, 25-fold, 20-fold, 15-fold, 10-fold, 5-fold, or 3-fold. In some embodiments, PPARα/γ dual agonist exhibits significant PPARα and/or PPARγ agonism, wherein the half-maximal concentration potencies (EC₅₀) for activation of hPPARγ and EC₅₀ for activation of hPPARα differ by more than 30-fold, 25-fold, 20-fold, 15-fold, 10-fold, 5-fold, or 3-fold.

In certain embodiments, the dual agonist of PPARα and PPARγ is Compound 2 or its crystalline form provided herein.

In another aspect, the present disclosure provides a method for modulating PPARα and/or PPARγ in a subject in need thereof, comprising administering an effective amount of the crystalline form of Compound 2 or the salt of Compound 2 of the present disclosure or the pharmaceutical composition of the present disclosure to the subject.

### EXAMPLES

For the purpose of illustration, the following examples are included. However, it is to be understood that these examples do not limit the present disclosure and are only meant to suggest a method of practicing the present disclosure. Persons skilled in the art will recognize that the chemical reactions described may be readily adapted to prepare a number of other compounds of the present disclosure, and alternative methods for preparing the compounds of the present disclosure are deemed to be within the scope of the present disclosure. For example, the synthesis of non-exemplified compounds according to the present disclosure may be successfully performed by modifications apparent to those skilled in the art, e.g., by appropriately protecting interfering groups, by utilizing other suitable reagents and building blocks known in the art other than those described, and/or by making routine modifications of reaction conditions. Alternatively, other reactions disclosed herein or known in the art will be recognized as having applicability for preparing other compounds of the present disclosure.

### Example 1: Synthesis of Compound 2

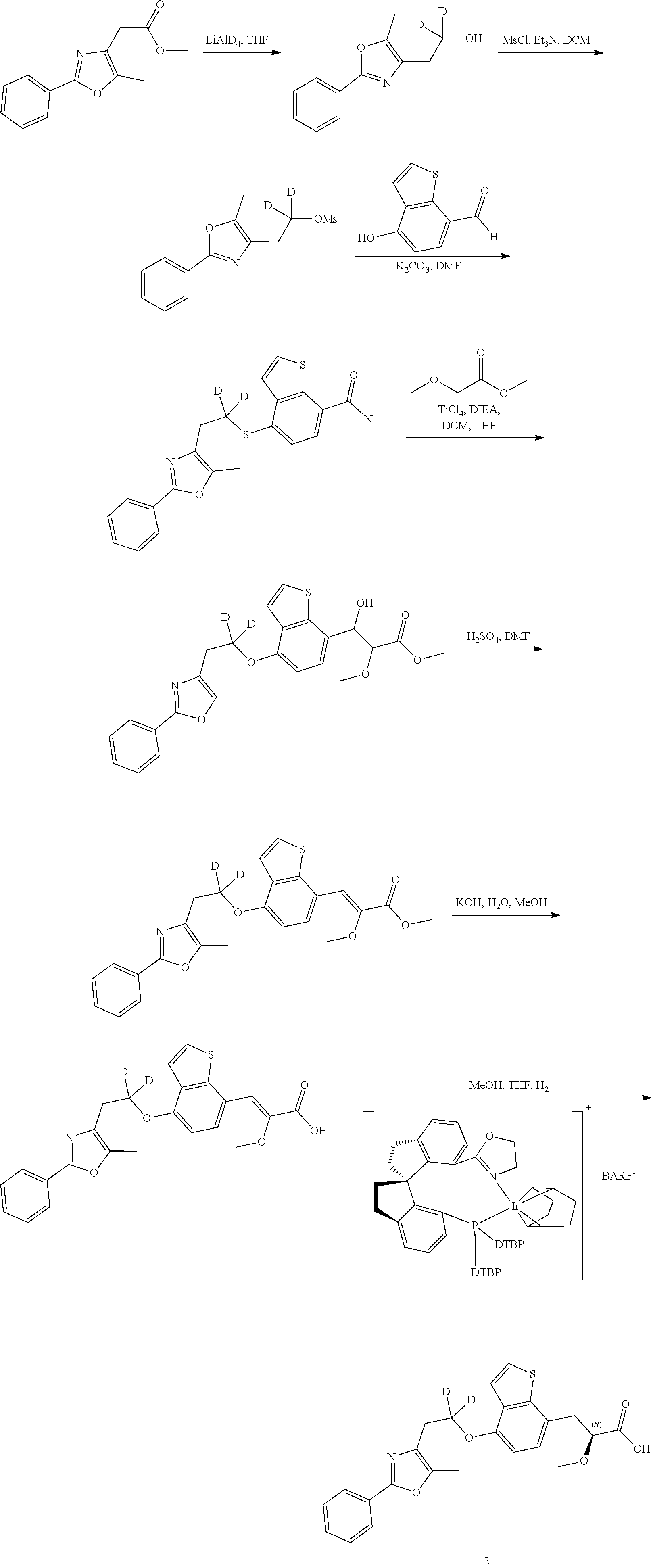

To a suspension of LiAlD4 (1.9 g, 45.4 mmol) in THF (40 mL) was added methyl 2-(5-methyl-2-phenyloxazol-4-yl)acetate (7.0 g, 30.3 mmol) in THF (60 mL) at 0° C. under N₂. The reaction was stirred at 0° C. for 2 hours, then quenched with water (3 mL). The resulting solid was filtered off. The filter cake was washed with EtOAc (500 mL), DCM/MeOH (10/1, 500 mL). The filtrate was concentrated under vacuum to afford 2-(5-methyl-2-phenyloxazol-4-yl) ethan-1,1-d₂₋₁-ol (5.0 g, yield 80.6%) as a yellow solid.

¹H NMR (400 MHz, CDCl₃) δ: 8.05-7.87 (m, 1H), 7.50-7.33 (m, 2H), 2.71 (s, 2H), 2.34 (s, 3H).

LC-MS (ESI⁺): m/z=206.2 ([M+H]⁺).

To a solution of 2-(5-methyl-2-phenyloxazol-4-yl) ethan-1,1-d₂₋₁-ol (5.0 g, 24.4 mmol) in DCM (100 mL) was added Et₃N (5.4 g, 53.7 mmol). The reaction mixture was cooled to 0° C., MsCl (5.6 g, 48.8 mmol) was added under N₂. The reaction was stirred at 0° C. for 2 hours, then poured into water. 1 N HCl (40 mL) was added and the mixture was extracted with DCM (100 mL×2). The combined organic layer was dried over Na₂SO₄, filtered and concentrated under vacuum. The residue was purified by column chromatography (Petroleum ether:EtOAc=20:1 to 10:1) to give 2-(5-methyl-2-phenyloxazol-4-yl)ethyl-1,1-d₂ methanesulfonate (5.0 g, yield 72.5%) as a white solid.

¹H NMR (400 MHz, CDCl₃) δ: 7.97 (dd, J=7.4, 2.2 Hz, 2H), 7.53-7.34 (m, 3H), 3.04-2.90 (m, 5H), 2.36 (s, 3H).

LC-MS (ESI⁺): m/z=284.0 ([M+H]).

To a solution of 4-hydroxybenzo[b]thiophene-7-carbaldehyde (3.2 g, 17.7 mmol) in DMF (30 mL) was added K₂CO₃ (2.9 g, 21.2 mmol). The reaction mixture was heated to 85° C. under N₂. 2-(5-methyl-2-phenyloxazol-4-yl)ethyl-1,1-d₂ methanesulfonate (5.0 g, 17.7 mmol) in DMF (15 mL) was added dropwise at this temperature. The reaction was stirred at 85° C. for 5 hours, then it was cooled to room temperature and poured into water, extracted with EtOAc (300 mL×2). The organic layer was washed with brine, dried over Na₂SO₄, filtered and concentrated. The residue was triturated with Petroleum ether/EtOAc=5/1 to give 4-(2-(5-methyl-2-phenyloxazol-4-yl) ethoxy-1,1-d₂)benzo[b]thiophene-7-carbaldehyde (5.7 g, yield 88.2%) as a brown solid.

¹H NMR (300 MHz, DMSO-d₆) δ: 10.05 (s, 1H), 8.07 (d, J=8.2 Hz, 1H), 8.00-7.86 (m, 2H), 7.82 (d, J=5.5 Hz, 1H), 7.62-7.38 (m, 4H), 7.23 (d, J=8.1 Hz, 1H), 3.06 (s, 2H), 2.40 (s, 3H).

LC-MS (ESI⁺): m/z=366.0 ([M+H]⁺).

To a solution of methyl 2-methoxyacetate (5.9 g, 57.2 mmol) in THF (40 mL) was added TiCl₄ (10.8 g, 57.2 mmol) at 0° C. under argon. The yellow solution was stirred at 0° C. for 15 min and DIEA (7.9 g, 61.6 mmol) was added. The black solution was stirred for another 15 min and 4-(2-(5-methyl-2-phenyloxazol-4-yl) ethoxy-1,1-d₂)benzo[b]thiophene-7-carbaldehyde (4.0 g, 11.0 mmol) in DCM (60 mL) was added dropwise. The reaction was stirred at 0° C. for 1 hour and warmed to room temperature overnight. Then it was cooled to 0° C. and quenched with water, extracted with DCM (200 mL×2). The organic layer was washed with brine, dried over Na₂SO₄, filtered and concentrated to give the crude methyl 3-hydroxy-2-methoxy-3-(4-(2-(5-methyl-2-phenyloxazol-4-yl) ethoxy-1,1-d₂)benzo[b]thiophen-7-yl) propanoate (7.7 g), which was directly used to the next step without further purification.

LC-MS (ESI⁺): m/z=470.2 ([M+H]⁺).

To a solution of methyl 3-hydroxy-2-methoxy-3-(4-(2-(5-methyl-2-phenyloxazol-4-yl) ethoxy-1,1-d₂)benzo[b]thiophen-7-yl) propanoate (7.7 g, crude) in DMF (40 mL) was added con. H₂SO₄ (10 mL) dropwise at ambient temperature. The reaction was stirred at 100° C. overnight, then it was diluted with EtOH (40 mL) and stirred at 0° C. for 1 hour. The solid was filtered, washed with EtOH (10 mL) and water (50 mL). The wet cake was dried to give methyl (Z)-2-methoxy-3-(4-(2-(5-methyl-2-phenyloxazol-4-yl) ethoxy-1,1-d₂)benzo[b]thiophen-7-yl) acrylate (2.3 g, 46.4% yield of 2 steps) as a yellow solid.

¹H NMR (400 MHz, CDCl₃) δ: 8.09 (d, J=8.4 Hz, 1H), 7.99 (dd, J=7.6, 1.8 Hz, 2H), 7.53-7.37 (m, 4H), 7.34 (d, J=5.5 Hz, 1H), 7.21 (s, 1H), 6.85 (d, J=8.4 Hz, 1H), 3.88 (s, 3H), 3.77 (s, 3H), 3.08 (s, 2H), 2.40 (s, 3H).

LC-MS (ESI⁺): m/z=452.2 ([M+H]⁺).

To a solution of methyl (Z)-2-methoxy-3-(4-(2-(5-methyl-2-phenyloxazol-4-yl) ethoxy-1,1-d₂)benzo[b]thiophen-7-yl) acrylate (2.3 g, 5.1 mmol) in MeOH (50 mL) was added KOH (1.7 g, 30.6 mmol) in H₂O (5 mL) at room temperature. The reaction was stirred at 80° C. for 2 hours. The reaction mixture was cooled to room temperature, diluted with H₂O (50 mL) and adjusted pH=3 with 6 N HCl. The mixture was cooled to 0° C. and the solid was filtered. The filter cake was suspended in EtOH (40 mL) at 80° C. for 1 hour, cooled to 0° C. and stirred for 1 hour. The solid was filtered and dried to afford (Z)-2-methoxy-3-(4-(2-(5-methyl-2-phenyloxazol-4-yl) ethoxy-1,1-d₂)benzo[b]thiophen-7-yl) acrylic acid (1.5 g, yield 68.2%) as a brown solid.

¹H NMR (400 MHz, CDCl₃) δ: 8.09 (d, J=8.4 Hz, 1H), 8.00 (dd, J=7.6, 1.9 Hz, 2H), 7.49 (d, J=5.5 Hz, 1H), 7.48-7.39 (m, 3H), 7.35 (t, J=2.7 Hz, 2H), 6.87 (d, J=8.4 Hz, 1H), 3.78 (s, 3H), 3.10 (s, 2H), 2.41 (s, 3H).

LC-MS (ESI⁺): m/z=438.2 ([M+H]⁺).

A 300 mL stainless steel autoclave was charged with (Z)-2-methoxy-3-(4-(2-(5-methyl-2-phenyloxazol-4-yl) ethoxy-1,1-d₂)benzo[b]thiophen-7-yl) acrylic acid (1.5 g, 3.4 mmol), (S)-phenylethylamine (82 mg, 0.68 mmol), MeOH (18 mL), THF (12 mL) and Ir-cat ([(S)-DTBSIPHOX)Ir(COD)]BArF, 9.3 mg, 0.001 eq). The autoclave was sealed and the hydrogenation was stirred at 70° C. under 30 bar of hydrogen for 16 hours. LCMS showed about half of starting material was remained, Ir-cat (10.3 mg) was added and the reaction was stirred for another 1 day. After the autoclave was opened and the yellowish solution was rotary evaporated to dryness (45° C.). The crude product was dissolved in EtOAc (150 mL) and was washed with 1 N HCl (40 mL×2). The organic layer was dried over Na₂SO₄, filtered and evaporated to dryness. The crude product was dissolved in isopropyl acetate at reflux and allowed to cool to 0° C. whereby crystallization started. The formed crystals were filtered off, washed with isopropyl acetate (50 mL) and dried to afford a yellow solid (920 mg), which was further purified by prep-HPLC (0.1% FA in CH₃CN and water) to give compound 2 (518 mg, yield 34.5%) as a white solid.

¹H NMR (400 MHz, CDCl₃) δ: 7.99 (dd, J=6.4, 2.4 Hz, 2H), 7.48 (d, J=5.6 Hz, 1H), 7.43-7.41 (m, 3H), 7.32 (d, J=5.6 Hz, 1H), 7.15 (d, J=8.0 Hz, 1H), 6.73 (d, J=8.0 Hz, 1H), 4.20 (dd, J=7.9, 4.7 Hz, 1H), 3.39-3.28 (m, 4H), 3.23-3.18 (m, 1H), 3.05 (s, 2H), 2.40 (s, 3H).

LC-MS (ESI⁺): m/z=440.2 ([M+H]⁺).

Chiral HPLC (Chiralpak AD-3 4.6 mm*250 mm 3 μm, 90% hexanes/9.99% EtOH/0.01% TFA, 210 nm): 99.57% ee.

### Example 2: Preparation of Crystalline Forms of Compound 2

### 2.1 Abbreviation for Solvents Used

The following abbreviations have been used in the examples:

TABLE 1
Solvent abbreviation list

| Abbreviation | Solvent |
|---|---|
| MeOH | Methanol |
| EtOH | Ethanol |
| IPA | Isopropyl alcohol |
| MIBK | 4-Methyl-2-pentanone |
| EtOAc | Ethyl acetate |
| IPAc | Isopropyl acetate |
| MTBE | Methyl tert-butyl ether |
| THF | Tetrahydrofuran |
| 2-MeTHF | 2-Methyltetrahydrofuran |
| ACN | Acetonitrile |
| CHCl₃ | Trichloromethane |
| DCM | Dichloromethane |
| DMAc | N,N-Dimethylacetamide |
| DMSO | Dimethylsulfoxide |
| NMP | N-Methyl pyrrolidone |
| MEK | Methylethyl ketone |
| CPME | Cyclopentyl methyl ether |
| / | / |

### 2.2 Characterization of Starting Materials

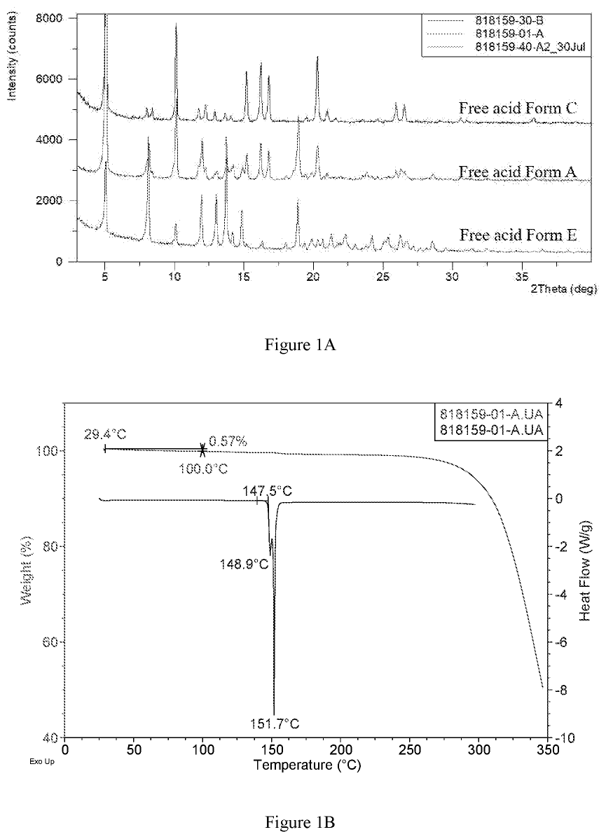

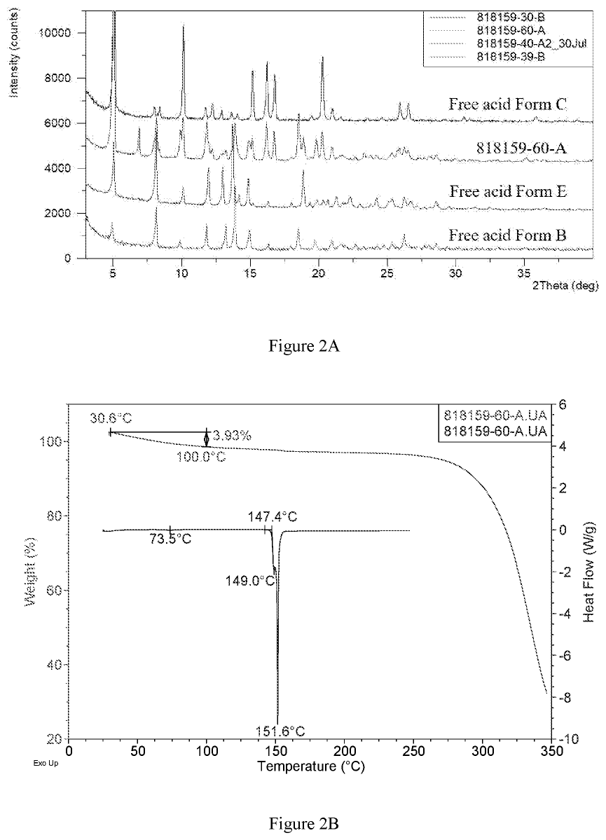

A total of two batches of AP-303 (i.e., Compound 2) free acid (batch No.: 01321040801) were received, respectively, and then characterized by XRPD, TGA and DSC. The results are summarized in Table 2. After further comparison, the first batch free acid material (818159-01-A) was a mixture of free acid Form C and E. The second batch free acid material (818159-60-A) was a mixture of free acid Form B/C/E. The XRPD patterns are displayed in FIG. 1A and FIG. 2A, respectively.

As shown in FIG. 1B, for the first batch free acid material, TGA result showed a weight loss of 0.9% up to 100° C., and DSC result showed two endothermic peaks at 148.9 and 151.7° C. (peak temperature). As shown in FIG. 2B, for the second batch free acid material, TGA result showed a weight loss of 3.9% up to 100° C., and DSC result showed three endothermic peaks at 73.5, 149.0 and 151.6° C. (peak temperature).

TABLE 2
Information and characterization results summary of starting materials

| | | | | TGA | Endotherm |
|---|---|---|---|---|---|
| Sample | | CP ID | | Weight (%, | (° C., |
| Name | Batch | (818159-) | Form | 100° C.) | peak) |
| AP-303 | 01321040801 | 01-A | Free acid | 0.6 | 148.9, 151.7 |
| | | | Form | | |
| | | | C + E | | |
| | 01321040801 | 60-A | Free acid | 3.9 | 73.5, 149.0, |
| | | | Form | | 151.6 |
| | | | C + E + B | | |

Approximate solubility of AP-303 (818159-01-A) were determined in 14 single solvents at RT (25±3° C.). Approximately 2 mg of sample was added into a 3-mL glass vial. Corresponding solvents were added stepwise (50 μL→50 μL→100 μL→200 μL→600 μL→1000 μL) into the vials until the solids were dissolved visually or a total volume of 2 mL was reached. The ranges of approximate solubility were calculated according to the mass of sample, volume of solvent and observation. Result summarized in Table 3 was used to guide the solvent selection in polymorph screening.

TABLE 3
Approximate solubility of AP-303 (818159-01-A) at RT

| Solvent | Solubility (mg/mL) | Solvent | Solubility (mg/mL) |
|---|---|---|---|
| MeOH | 1.9 < S < 4.8 | THF | S > 42.0 |
| EtOH | 4.8 < S < 9.5 | 1,4-Dioxane | S > 46.0 |
| IPA | 2.0 < S < 5.0 | ACN | 2.0 < S < 5.0 |
| Acetone | 12.5 < S < 25.0 | DCM | 11.5 < S < 23.0 |
| MEK | 19.0 < S < 38.0 | n-Heptane | S < 1.3 |
| EtOAc | 10.0 < S < 20.0 | DMSO | S > 44.0 |
| MTBE | 2.1 < S < 5.3 | H₂O | S < 1.2 |

### 2.3 Polymorph Screening Experiments

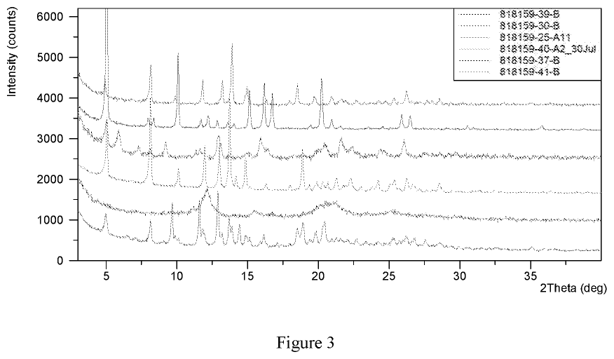

Polymorph screening was performed under 100 experimental conditions starting with free acid (818159-01-A) material. A total of 10 methods were used, including anti-solvent addition/reverse anti-solvent addition, slurry at RT, slurry at 50° C., solid vapor diffusion, liquid vapor diffusion, slow evaporation, slow cooling, temperature cycling, polymer-induced crystallization and humidity induced crystallization. The results of polymorph screening are summarized in Table 4. As XRPD results shown in FIG. 3, six free acid forms were obtained, namely free acid Form B~G.

TABLE 4
Summary of polymorph screening

| | No. of | |
|---|---|---|
| Method | Experiments | Crystal Form |
| Slow cooling | 6 | Free acid Form E/F, E + C, |
| | | B + peaks, low crystallinity |
| Liquid vapor | 6 | Free acid Form F, B + E, |
| diffusion | | amorphous, low crystallinity |
| Anti-solvent | 12 | Free acid Form C/E/F/G, B + C + E, |
| addition | | C + E, amorphous, low crystallinity |
| Reverse anti- | 8 | Free acid Form C/E/F, B + E, |
| solvent addition | | amorphous, low crystallinity |
| Slow evaporation | 5 | Free acid Form C/F, B + E, E + peaks |
| Slurry at 50° C. | 17 | Free acid Form E, E + peaks, E + B |
| Temperature | 9 | Free acid Form B + E |
| cycling | | |
| Solid vapor | 8 | Free acid Form E + B + C, B + E, |
| diffusion | | C + E, F + peaks |
| Humidity induced | 5 | Free acid Form C + E + B |
| Polymer-induced | 7 | Free acid Form E/F, B + E |
| crystallization | | |
| Slurry at RT | 17 | Free acid Form B/D/E, B + E |
| Total | 100 | Free acid Form B/C/D/E/F/G, |
| | | B + E, B + peaks, E + peaks, |
| | | F + peaks, C + E, B + C + E |
| | | amorphous, low crystallinity |

### 2.3.1 Slow Cooling

Slow cooling experiments were conducted in 6 solvent systems. About 20 mg of starting material (818159-01-A) was dissolved in 1.0 mL of solvent or solvent mixture at 50° C. and filtered to a new vial using a 0.45 μm PTFE membrane. Filtrates were slowly cooled down from 50° C. to 5° C. at a rate of 0.05° C./min. The obtained solids were kept isothermal at 5° C. or −20° C. before isolated for XRPD analysis. Slow evaporation was conducted if no solid observed. Result summarized in Table 5 shows that free acid Form E/F, Form E+C, Form B+peaks (unidentified peaks except Form B) and low crystallinity were obtained.

TABLE 5
Summary of slow cooling experiments

| Sample ID | Solvent (v/v) | Results |
|---|---|---|
| 818159-15-A1 | EtOH | Low crystallinity |
| 818159-15-A2 | ACN | Free acid Form B + peaks |
| 818159-15-A3 | Acetone/H₂O (1:3) | Limited solids |
| 818159-15-A4 | EtOAc/ | Free acid Form |
| | Methylcyclohexane (2:1) | E (major) + C |
| 818159-15-A5 | 2-Me THF/n-heptane (1:1) | Free acid Form F |
| 818159-15-A6 | CHCl₃/toluene (1:1) | Free acid Form E |

### 2.3.2 Liquid Vapor Diffusion

Liquid vapor diffusion experiments were conducted under 6 conditions. About 20 mg of starting material (818159-01-A) was dissolved in 0.2~0.8 mL solvent to obtain a clear solution in a 3-mL vial and filtered to a new vial using a 0.45 μm PTFE membrane. Filtrate was transferred into a 3-mL vial, which was then placed into a 20-mL vial with 3 mL of volatile solvents. The 20-mL vial was sealed with a cap and kept at RT allowing sufficient time for organic vapor to interact with the solution. If the samples were still clear, slow evaporation was conducted at RT. The precipitates were isolated for XRPD analysis. The result summarized in Table 6 shows that free acid Form F, Form B+E, low crystallinity and amorphous were obtained.

TABLE 6
Summary of liquid vapor diffusion experiments

| Sample ID | Solvent | Anti-solvent | Results |
|---|---|---|---|
| 818159-16-A1 | DMSO | H₂O | Amorphous |
| 818159-16-A2 | | MTBE | Low crystallinity |
| 818159-16-A3 | MEK | MTBE | Free acid Form B + E |
| 818159-16-A4 | | n-Heptane | Free acid Form B + E |
| 818159-16-A5 | 1,4- | H₂O | Free acid Form F |
| 818159-16-A6 | dioxane | n-Pentane | Free acid Form F |

### 2.3.3 Anti-Solvent Addition

Anti-solvent addition experiments were conducted under 12 conditions. About 20 mg of starting material (818159-01-A) was dissolved in 0.4~1.0 mL solvent to obtain a clear solution. The solution was magnetically stirred followed by addition of 0.2 mL anti-solvent per step until precipitate appeared or the total amount of anti-solvent reached 5 mL. Clear solutions were transferred to stir at 5° C. or −20° C. to induce precipitate. If the samples were still clear, slow evaporation was conducted at RT. The obtained precipitate was isolated for XRPD analysis. Result in Table 7 shows that free acid Form C/E/F/G, Form C+E, Form B+C+E, amorphous and low crystallinity were observed.

TABLE 7
Summary of anti-solvent addition experiments

| Sample ID | Solvent | Anti-solvent | Results |
|---|---|---|---|
| 818159-17-A1 | DMSO | H₂O | Amorphous |
| 818159-17-A2 | | MTBE | Free acid Form G |
| 818159-17-A3 | MEK | m-Xylene | Free acid Form C |
| 818159-17-A4 | | n-Heptane | Free acid Form E |
| 818159-17-A5 | EtOAc | n-Hexane | Free acid Form E |
| 818159-17-A6 | | MTBE | Free acid Form |
| | | | B + C + E |
| 818159-17-A7 | 2-Me THF | CPME | Free acid Form F |
| 818159-17-A8 | | Toluene | Free acid Form C+E |
| 818159-17-A9 | 1,4-dioxane | H₂O | Low crystallinity |
| 818159-17-A10 | | Toluene | Free acid Form C |
| 818159-17-A11 | DMAc | H₂O | Amorphous |
| 818159-17-A12 | NMP | H₂O | Amorphous |

### 2.3.4 Reverse Anti-Solvent Addition

Reverse anti-solvent addition experiments were conducted under 8 conditions, respectively. About 20 mg of starting material (818159-01-A) was dissolved in 0.4~1.0 mL solvent to obtain a clear solution. The solution was added to anti-solvent. Clear solutions were transferred to stir at 5° C. or −20° C. to induce precipitate. If the samples were still clear, slow evaporation was conducted at RT. The obtained precipitate was isolated for XRPD analysis. Result in Table 8 shows that free acid Form C/E/F, Form B+E, amorphous and low crystallinity were observed.

TABLE 8
Summary of reverse anti-solvent addition experiments

| Sample ID | Anti-solvent | Solvent | Results |
|---|---|---|---|
| 818159-18-A1 | n-heptane | DCM | Free acid Form B + E |
| 818159-18-A2 | | EtOAc | Free acid Form E |
| 818159-18-A3 | | THF | Free acid Form E |
| | | | (major) + B |
| 818159-18-A4 | Toluene | Acetone | Free acid Form C |
| 818159-18-A5 | | EtOAc | Free acid Form F |
| 818159-18-A6 | | CHCl₃ | Low crystallinity |
| 818159-18-A7 | H₂O | ACN | Low crystallinity |
| 818159-18-A8 | | DMF | Amorphous |

### 2.3.5 Slow Evaporation

Slow evaporation experiments were conducted in 5 solvent systems. About 20 mg of starting material (818159-01-A) was dissolved in 0.6~3.0 mL of solvent or solvent mixture and filtered to a new vial using a 0.45 μm PTFE membrane. Filtrates were slowly evaporated at RT and the obtained solids were isolated for XRPD analysis. Result summarized in Table 9 shows that free acid Form C/F, Form B+E and Form E+peaks were obtained.

TABLE 9
Summary of slow evaporation experiments

| Sample ID | Solvent (v/v) | Results |
|---|---|---|
| 818159-19-A1 | Acetone | Free acid Form B + E |
| 818159-19-A2 | EtOAc | Free acid Form F |
| 818159-19-A3 | DCM | Free acid Form C |
| 818159-19-A4 | MTBE | Free acid Form E + peaks |
| 818159-19-A5 | THF/H₂O (4:1) | Free acid Form F |

### 2.3.6 Slurry at 50° C.

Slurry conversion experiments were conducted at 50° C. in 17 solvent systems. About 20 mg of starting material (818159-01-A) was suspended in 0.5 mL of solvent at 50° C. for 2 days. The remaining solids were isolated for XRPD analysis. Result summarized in Table 10 indicated that free acid Form E, Form B+E, Form E+C and Form E+peaks were obtained.

TABLE 10
Summary of slurry conversion experiments at 50° C.

| Sample ID | Solvent (v/v) | Results |
|---|---|---|
| 818159-20-A1 | m-xylene | Free acid Form E (major) + B |
| 818159-20-A2 | Cumene | Free acid Form E (major) + B |
| 818159-20-A3 | n-heptane | Free acid Form E (major) + C |
| 818159-20-A4* | CPME | Free acid Form E + B |
| 818159-20-A5 | MIBK | Free acid Form E |
| 818159-20-A6 | IPA | Free acid Form E (major) + B |
| 818159-20-A7 | Isobutanol | Free acid Form E (major) + B |
| 818159-20-A8 | ACN | Free acid Form E + B |
| 818159-20-A9 | THF/n-Heptane (1:4) | Free acid Form E |
| 818159-20-A10 | IPAc/Toluene (1:3) | Free acid Form E (major) + B |
| 818159-20-A11 | 1,4-dioxane/H₂O (1:9) | Free acid Form E (major) + B |
| 818159-20-A12 | CHCl₃/m-Xylene (1:4) | Free acid Form E (major) + B |
| 818159-20-A13 | ACN/H₂O (a_{w}~0.2, | Free acid Form E (major) + B |
| | 0.99: 0.01) | |
| 818159-20-A14 | ACN/H₂O (a_{w}~0.4, | Free acid Form B + E |
| | 0.978: 0.022) | |
| 818159-20-A15 | ACN/H₂O (a_{w}~0.6, | Free acid Form B + E |
| | 0.96: 0.04) | |
| 818159-20-A16 | ACN/H₂O (a_{w}~0.8, | Free acid Form E (major) + B |
| | 0.925: 0.075) | |
| 818159-20-A17 | H₂O | Free acid Form E + peaks |

*The sample was transferred to 5° C. for 6 days due to limited solids; a_{w}: water activity.

### 2.3.7 Temperature Cycling

Temperature cycling experiments were conducted under 9 conditions, respectively. About 20 mg of starting material (818159-01-A) was suspended in 0.5 mL of solvent. Then the suspensions were magnetically stirred from 50° C. to 5° C. for two cycles with a heating/cooling rate at 0.1° C./min. The remaining solids were isolated for XRPD analysis. Result summarized in Table 11 showed that free acid Form B+E was obtained.

TABLE 11
Summary of temperature cycling experiments

| Sample ID | Solvent (v/v) | Results |
|---|---|---|
| 818159-21-A1 | Toluene | Free acid Form B |
| | | (major) + E |
| 818159-21-A2 | n-Hextane | Free acid Form E |
| | | (major) + B |
| 818159-21-A3 | P-Cymene | Free acid Form E + B |
| 818159-21-A4 | Acetone/H₂O (1:2) | Free acid Form B + E |
| 818159-21-A5 | NMP/H₂O (1:4) | Free acid Form B + E |
| 818159-21-A6 | IPA/n-Heptane (1:1) | Free acid Form B |
| | | (major) + E |
| 818159-21-A7 | CHCl₃/n-Heptane (1:3) | Free acid Form B |
| | | (major) + E |
| 818159-21-A8 | MEK/m-Xylene(1:9) | Free acid Form B |
| | | (major) + E |
| 818159-21-A9 | MeOH/MTBE (1:1) | Free acid Form E |
| | | (major) + B |

### 2.3.8 Solid Vapor Diffusion

Solid vapor diffusion experiments were conducted using 8 different solvents systems. About 20 mg of starting material (818159-01-A) was weighed into a 3-mL vial, which was placed into a 20-mL vial with 3.0 mL of volatile solvent. The 20-mL vial was sealed with a cap and kept at RT for 10 days allowing solvent vapor to interact with sample. The solids were tested by XRPD. Result summarized in Table 12 shows that free acid Form E+B, Form C+E, Form C+E+B and Form F+ peaks were obtained.

TABLE 12
Summary of solid vapor diffusion experiments

| Sample ID | Solvent | Results |
|---|---|---|
| 818159-22-A1 | Toluene | Free acid Form C + E |
| 818159-22-A2 | ACN | Free acid Form B + E + C |
| 818159-22-A3 | MeOH | Free acid Form E + B |
| 818159-22-A4 | EtOH | Free acid Form B + E + C |
| 818159-22-A5 | EtOAc | Free acid Form B + E + C |
| 818159-22-A6* | THF | Free acid Form F + peaks |
| 818159-22-A7 | Acetone | Free acid Form B + E + C |
| 818159-22-A8 | DMSO | Free acid Form B + E |

*Solids were obtained after evaporation at RT.

### 2.3.9 Humidity Induced Crystallization

Humidity induced crystallization experiments were conducted under 5 conditions, respectively. About 20 mg of starting material (818159-01-A) was weighed into a 3-mL vial, which was placed into a 20-mL vial with 2 mL of saturated inorganic salt aqueous solution. The 20-mL vial was sealed with a cap and kept at RT for 9 days allowing water vapor to interact with sample. The solids were tested by XRPD. The result summarized in Table 13 shows that free acid Form C+E+B was obtained.

TABLE 13
Summary of humidity induced crystallization experiments

| Sample ID | Solvent | Results |
|---|---|---|
| 818159-23-A1 | ~20% RH | Free acid Form C + E + B |
| 818159-23-A2 | ~40% RH | Free acid Form C + E + B |
| 818159-23-A3 | ~60% RH | Free acid Form C + E + B |
| 818159-23-A4 | ~80% RH | Free acid Form C + E + B |
| 818159-23-A5 | ~100% RH | Free acid Form C + E + B |

### 2.3.10 Polymer Induced Crystallization

Polymer induced crystallization experiments were performed with two sets of polymer mixtures in different solvent systems. About 20 mg of starting material (818159-01-A) was dissolved in 0.4-2.0 mL of solvent in a 3-mL glass vial. About 2 mg of polymer mixture was added into the 3-mL glass vial. All solutions and suspensions were filtered using a 0.45 μm PTFE membrane. The resulting solutions were subjected to evaporation at RT with vials sealed by Parafilm® for slow evaporation. The solids were isolated for XRPD analysis. Results summarized in Table 14 shows that free acid Form E/F and Form B+E were obtained.

TABLE 14
Summary of polymer induced crystallization experiments

| Sample ID | Solvent | Polymer | Results |
|---|---|---|---|
| 818159-24-A1 | EtOH | A | Free acid Form E |
| 818159-24-A2 | MEK | | Free acid Form F* |
| 818159-24-A3 | CHCl₃ | | Free acid Form E (major) + B |
| 818159-24-A4 | THF/H₂O (4:1) | | Free acid Form F |
| 818159-24-A5 | Acetone | B | Free acid Form B + E |
| 818159-24-A6 | 2-Me THF | | Free acid Form F |
| 818159-24-A7 | EtOAc | | Free acid Form F |

Polymer mixture A: polyvinyl pyrrolidone (PVP), polyvinyl alcohol (PVA), polyvinylchloride (PVC), polyvinyl acetate (PVAC), hypromellose (HPMC), methyl cellulose (MC) (mass ratio of 1:1:1:1:1:1).
Polymer mixture B: polycaprolactone (PCL), polyethylene glycol (PEG), polymethyl methacrylate (PMMA) sodium alginate (SA), and hydroxyethyl cellulose (HEC) (mass ratio of 1:1:1:1:1).
*color change to red brown.

### 2.3.11 Slurry at RT

Slurry conversion experiments were conducted at RT in 17 solvent systems. About 20 mg of starting material (818159-01-A) was suspended in 0.5 mL of solvent. The samples were stirred at RT for 7 days. The remaining solids were isolated for XRPD analysis. Result summarized in Table 15 shows that free acid Form B/D/E and Form B+E were obtained.

TABLE 15
Summary of slurry conversion experiments at RT

| Sample ID | Solvent (v/v) | Results |
|---|---|---|
| 818159-25-A1 | n-Pentane | Free acid Form B |
| | | (major) + E |
| 818159-25-A2 | MeOH | Free acid Form E |
| 818159-25-A3 | Cyclohexane | Free acid Form B |
| 818159-25-A4 | MTBE | Free acid Form B |
| 818159-25-A5 | Anisole | Free acid Form B |
| 818159-25-A6 | EtOH/H₂O(4:1) | Free acid Form B |
| 818159-25-A7 | DMF/H₂O (1:5) | Free acid Form B |
| | | (major) + E |
| 818159-25-A8 | EtOAc/n-Heptane (2:1) | Free acid Form B |
| 818159-25-A9 | Acetone/n-Heptane (1:4) | Free acid Form B |
| | | (major) + E |
| 818159-25-A10 | 2 Me THF/n-Hexane (1:3) | Free acid Form B |
| | | (major) + E |
| 818159-25-A11 | DCM/Cyclohexane (2:1) | Free acid Form D |
| 818159-25-A12 | MEK/Toluene (1:1) | Free acid Form B |
| 818159-25-A13 | MeOH/H₂O (a_{w}~0.2, 0.932: 0.065) | Free acid Form E |
| 818159-25-A14 | MeOH/H₂O (a_{w}~0.4, 0.84: 0.16) | Free acid Form E |
| 818159-25-A15 | MeOH/H₂O (a_{w}~0.6, 0.7: 0.3) | Free acid Form E |
| | | (major) + B |
| 818159-25-A16 | MeOH/H₂O (a_{w}~0.8, 0.42: 0.58) | Free acid Form E |
| | | (major) + B |
| 818159-25-A17 | H₂O | Free acid Form |
| | | B + E |

a_{w}: water activity.

### Example 3: Characterization of Crystalline Forms of Compound 2

### General Methods

### X-ray Powder Diffraction (XRPD)

For XRPD analysis, PANalytical X'pert 3 X-ray powder diffractometers were used. Sample was spread on the middle of a zero-background Si holder. The XRPD parameters used are listed in Table 16.

TABLE 16
Parameters for XRPD test

| Model | X′ Pert3 | Empyrean (VT) |
|---|---|---|
| X-Ray wavelength | Cu, Kα, | Cu, Kα, |
| | Kα1 (Å):1.540598, | Kα1 (Å):1.540598, |
| | Kα2 (Å):1.544426 | Kα2 (Å):1.544426 |
| | Kα2/Kα1 intensity | Kα2/Kα1 intensity |
| | ratio:0.50 | ratio:0.50 |
| X-Ray tube setting | 45 kV, 40 mA | 45 kV, 40 mA |
| Divergence slit | 1/8° | 1/8° |
| Scan mode | Continuous | Continuous |
| Scan range (°2TH) | 3-40 | 3-40 |
| Scan step time (s) | 46.7 | 46.7 |
| Step size (°2TH) | 0.0263 | 0.0167 |
| Test Time (s) | ~5 min | ~10 min |

### Thermal Gravimetric Analyses (TGA) and Differential Scanning Calorimetry (DSC)

TGA data were collected using a TA Q5000 TGA from TA Instruments and DSC was performed using a TA 2500 DSC from TA Instruments. Detailed parameters used are listed in Table 17.

TABLE 17
Parameters for TGA and DSC test

| Parameters | TGA | DSC |
|---|---|---|
| Method | Ramp | Ramp |
| Sample pan | Aluminum, open | Aluminum, crimped |
| Temperature | RT-desired temperature | 25° C.-desired temperature |
| Heating rate | 10° C./min | 10° C./min |
| Purge gas | N₂ | N₂ |

### Dynamic Vapor Sorption (DVS)

DVS was measured via a SMS (Surface Measurement Systems) DVS Intrinsic. The relative humidity at 25° C. were calibrated against deliquescence point of LiCl, Mg(NO₃)₂ and KCl. Parameters for DVS test were listed in Table 18.

TABLE 18
Parameters for DVS test

| Item | Value | |
|---|---|---|
| Temperature | 25° | C. |
| Sample size | 10~20 | mg |
| Gas and flow rate | N₂, 200 | mL/min |
| dm/dt | 0.002%/min | |
| Min. dm/dt stability duration | 10 | min |
| Max. equilibrium time | 180 | min |

| | |
|---|---|
| RH range | 0% RH-95% RH-0% RH |
| | 0% (0% RH-95% RH, 95% RH-0% RH) |
| RH step size | 30% (30% RH-95% RH, |
| | 95% RH-0% RH, 0% RH-90% RH) |
| | 40% (40% RH-95% RH, 95% |
| | RH-0% RH, 0% RH-90% RH) |

### Solution ¹H NMR

Solution proton NMR was collected on Bruker 400M NMR Spectrometer using DMSO-d6 or MeOD.

### HPLC/IC

An Agilent 1260 HPLC with DAD/VWD detector was utilized and detailed chromatographic conditions for HPLC analysis are listed in Table 19. IC method for counter-ion content measurement is listed in Table 20 below.

TABLE 19
Chromatographic conditions for purity and solubility analysis

| HPLC | Agilent 1260 Detector | |
|---|---|---|
| Column | Poroshell 120 EC C18, | |
| | 3.0 mm/50 mm/2.7 μm | |
| | A: 0.1% H₃PO₄ in H₂O | |
| | B: Acetonitrile | |
| | Time (min) | % B |
| Gradient table | 0.0 | 5 |
| | 5.0 | 95 |
| | 8.0 | 95 |
| | 8.1 | 5 |
| | 10.0 | 5 |
| Run time | 10 | min |
| Flow rate | 1.2 | mL/min |
| Injection volume | 3 | μL |
| Detector wavelength | UV at 210 nm | |
| Column temperature | 40° | C. |

| | |
|---|---|
| Sampler temperature | RT |
| Diluent | Acetonitrile/H₂O = 1:1 (v/v) |

TABLE 20
IC method for counter-ion content measurement

| IC | ThermoFisher ICS-1100 |
|---|---|
| Column | IonPac CS12A Analytical Column (4 × 250 mm) |
| Mobile Phase | 25 mM methanesulfonic acid |
| Injection volume | 25 μL |
| Flow rate | 1.0 mL/min |
| Cell temperature | 35° C. |
| Column temperature | 35° C. |
| Current | 80 mA |
| Run Time | 7.0 min |

### 3.1 Crystalline Form B of Compound 2

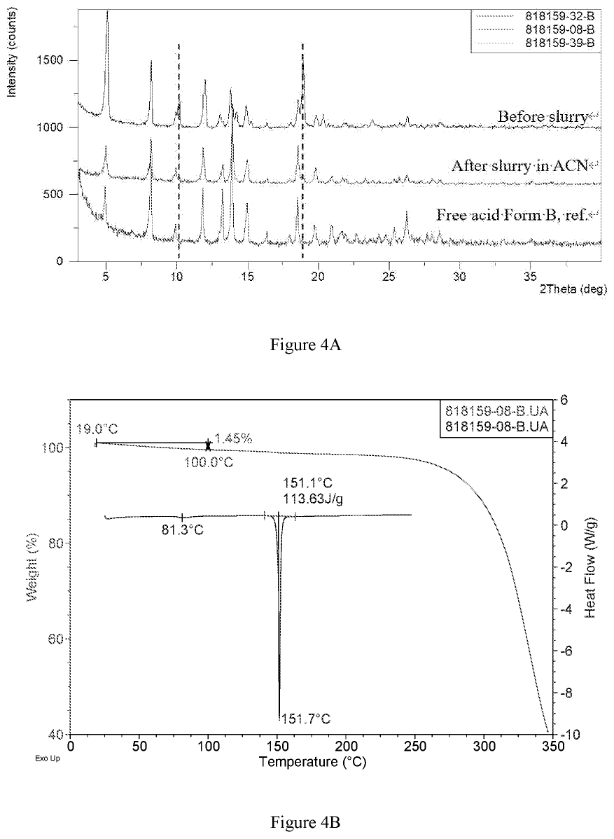

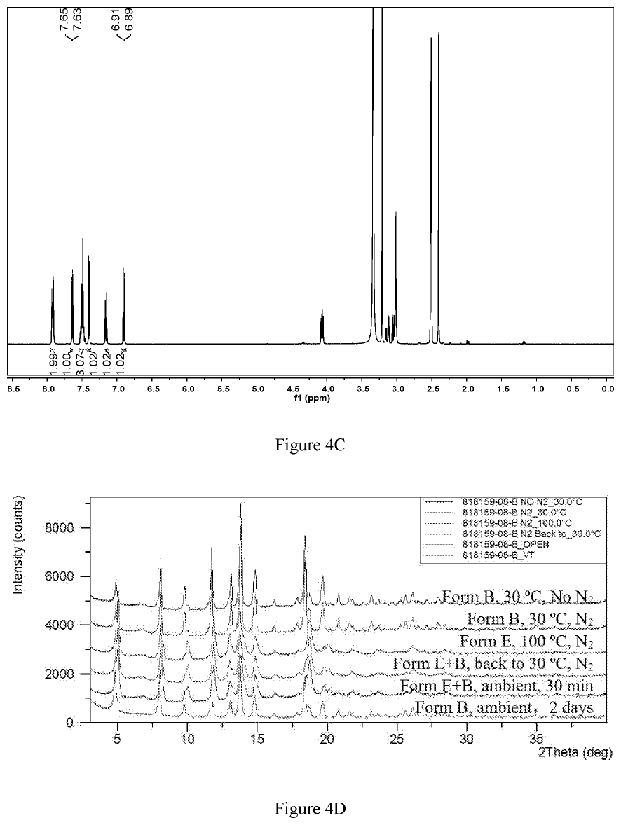

Free acid Form B (818159-08-B) was obtained via slurry starting material (818159-01-A) in EtOAc, as XRPD displayed in FIG. 4A. As shown in FIG. 4B, TGA result showed a weight loss of 1.5% up to 100° C., and DSC result showed two endothermic peaks at 81.3° C. (peak temperature) and 151.1° C. (onset temperature). No residual EtOAc was detected based on ¹H NMR results (FIG. 4C).

As XRPD displayed in FIG. 5A and Table 21, free acid Form B (818159-39-B) was purified via slurry free acid Form B (818159-08-B) in ACN at RT. As shown in FIG. 5B, TGA result showed a weight loss of 1.9% up to 100° C., and DSC result showed two endothermic peaks at 84.7° C. (peak temperature) and 150.4° C. (onset temperature).

To investigate form origin, variable-temperature XRPD (VT XRPD) experiment was conducted for free acid Form B (818159-08-B) as shown in FIG. 4D. VT XRPD results showed: 1) no form change was observed after N₂ bleeding at 30° C.; 2) it converted to free acid Form E after heating to 100° C. under N₂ protection; 3) it converted back to free acid Form B partially after cooling to 30° C. under N₂ protection; 4) it converted back to free acid Form B completely after storage at ambient for two days. Based on VT XRPD results, free acid Form B and Form E are speculated to be anhydrate. The endothermic peak at 84.7° C. (peak temperature) is speculated to relate to form transition and the endothermic peak at 150.4° C. (onset temperature) is consistent with that of Form E.

TABLE 21
XRPD of free acid Form B (818159-39-B)

| Pos. | Height | FWHM | d-spacing | Rel. |
|---|---|---|---|---|
| [2θ] | [cts] | Left [°2θ] | [Å] | Int. [%] |
| 4.94 | 284.19 | 0.1023 | 17.90 | 26.76 |
| 8.16 | 669.82 | 0.1023 | 10.84 | 63.07 |
| 9.89 | 125.94 | 0.1023 | 8.94 | 11.86 |
| 11.83 | 407.27 | 0.1023 | 7.48 | 38.35 |
| 13.23 | 420.41 | 0.1023 | 6.69 | 39.58 |
| 13.90 | 1062.12 | 0.1023 | 6.37 | 100.00 |
| 14.97 | 324.08 | 0.1279 | 5.92 | 30.51 |
| 16.29 | 56.10 | 0.3070 | 5.44 | 5.28 |
| 18.54 | 369.08 | 0.1279 | 4.79 | 34.75 |
| 19.74 | 140.37 | 0.2047 | 4.50 | 13.22 |
| 20.92 | 152.50 | 0.1791 | 4.25 | 14.36 |
| 21.63 | 96.56 | 0.3070 | 4.11 | 9.09 |
| 22.71 | 73.12 | 0.1535 | 3.92 | 6.88 |
| 25.35 | 109.61 | 0.1535 | 3.51 | 10.32 |
| 26.24 | 264.27 | 0.1023 | 3.40 | 24.88 |
| 28.55 | 84.82 | 0.2047 | 3.13 | 7.99 |

### 3.2 Crystalline Form C of Compound 2

Free acid Form C (818159-30-B) was obtained via slow evaporation in DCM. XRPD and TGA/DSC are displayed in FIG. 6A and Table 22, and FIG. 6B, respectively. TGA result showed a weight loss of 2.8% up to 100° C., and DSC result showed one endothermic peak at 147.6° C. (onset temperature) which was speculated to be melting point. ¹H NMR results (FIG. 6C) showed no residual DCM was detected. Considering low TGA weight loss and neat DSC, free acid Form C is speculated to be anhydrate.

TABLE 22
XRPD of free acid Form C (818159-30-B)

| Pos. | Height | FWHM | d-spacing | Rel. |
|---|---|---|---|---|
| [2θ] | [cts] | Left [°2θ] | [Å] | Int. [%] |
| 5.06 | 7154.61 | 0.0768 | 17.46 | 100.00 |
| 7.99 | 197.67 | 0.1023 | 11.06 | 2.76 |
| 8.37 | 224.95 | 0.0768 | 10.56 | 3.14 |
| 10.09 | 1847.99 | 0.1023 | 8.76 | 25.83 |
| 11.71 | 229.88 | 0.1023 | 7.56 | 3.21 |
| 12.22 | 307.46 | 0.1023 | 7.24 | 4.30 |
| 12.87 | 191.97 | 0.1023 | 6.88 | 2.68 |
| 13.60 | 149.74 | 0.1279 | 6.51 | 2.09 |
| 14.04 | 101.99 | 0.1535 | 6.31 | 1.43 |
| 15.15 | 961.53 | 0.1279 | 5.85 | 13.44 |
| 16.18 | 1184.01 | 0.1279 | 5.48 | 16.55 |
| 16.75 | 878.70 | 0.1279 | 5.29 | 12.28 |
| 19.43 | 78.83 | 0.1535 | 4.57 | 1.10 |
| 20.24 | 1292.21 | 0.1279 | 4.39 | 18.06 |
| 20.96 | 234.70 | 0.1023 | 4.24 | 3.28 |
| 25.89 | 367.01 | 0.1023 | 3.44 | 5.13 |
| 26.47 | 315.71 | 0.1791 | 3.37 | 4.41 |
| 30.56 | 92.37 | 0.1535 | 2.93 | 1.29 |
| 35.80 | 99.55 | 0.1535 | 2.51 | 1.39 |

### 3.3 Crystalline Form D of Compound 2

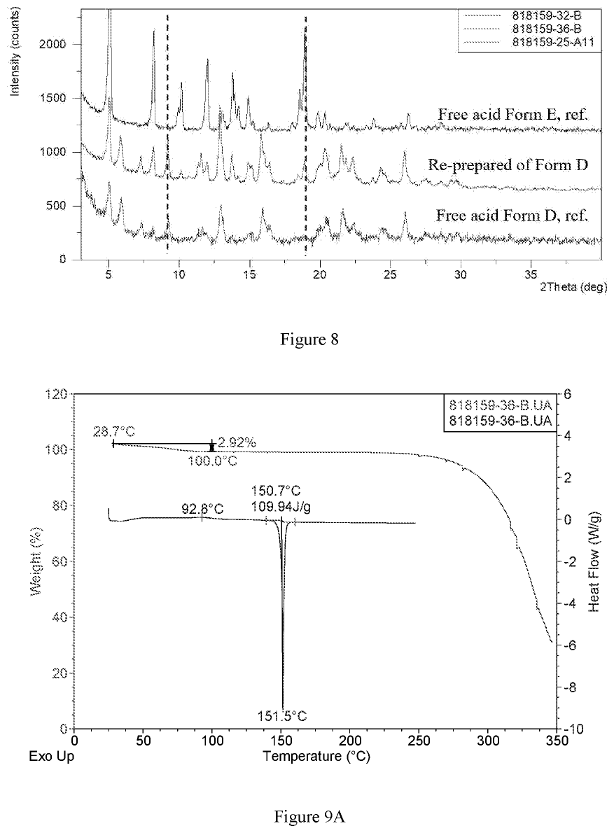

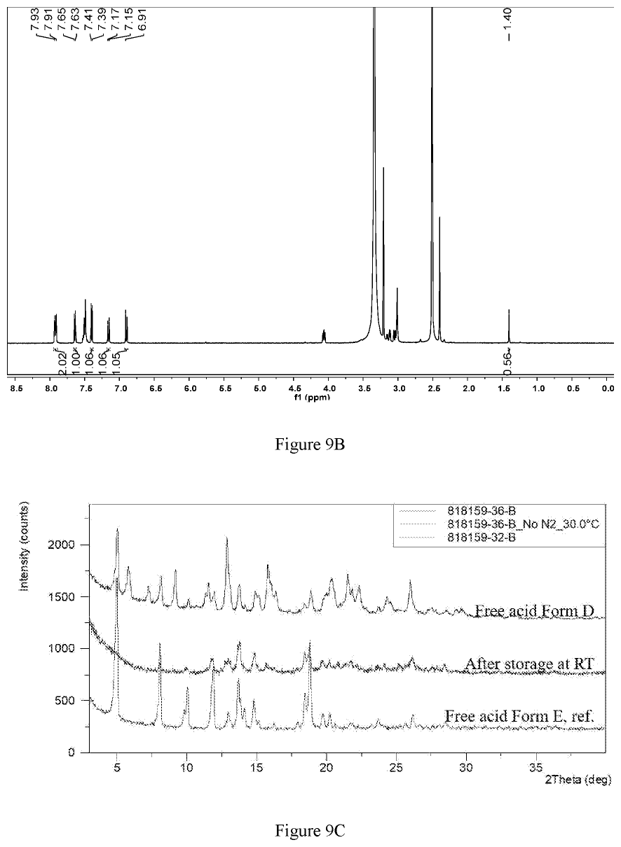

Free acid Form D (818159-25-A11) was obtained via slurry starting material (818159-01-A) in DCM/cyclohexane (2:1, v/v) at RT. XRPD results are displayed in FIG. 7 and Table 23. Free acid Form D (818159-36-B) was re-prepared using the same method for characterization. XRPD and TGA/DSC results are displayed in FIG. 8 and FIG. 9A. TGA result showed a weight loss of 2.9% up to 100° C., and DSC result (FIG. 9A) showed one endothermic peak at 150.7° C. (one temperature) and one exotherm at 92.8° C. (peak temperature). ¹H NMR results (FIG. 9B) showed the molar ratio of cyclohexane to API was 0.05 (0.9 wt %). As shown in FIG. 9C, it converted to free acid Form E after storage at RT for 1 month. Based on characterization results, free acid Form D is speculated be hygroscopic free acid anhydrate or hydrate (theoretical weight loss of monohydrate is 2.1%).

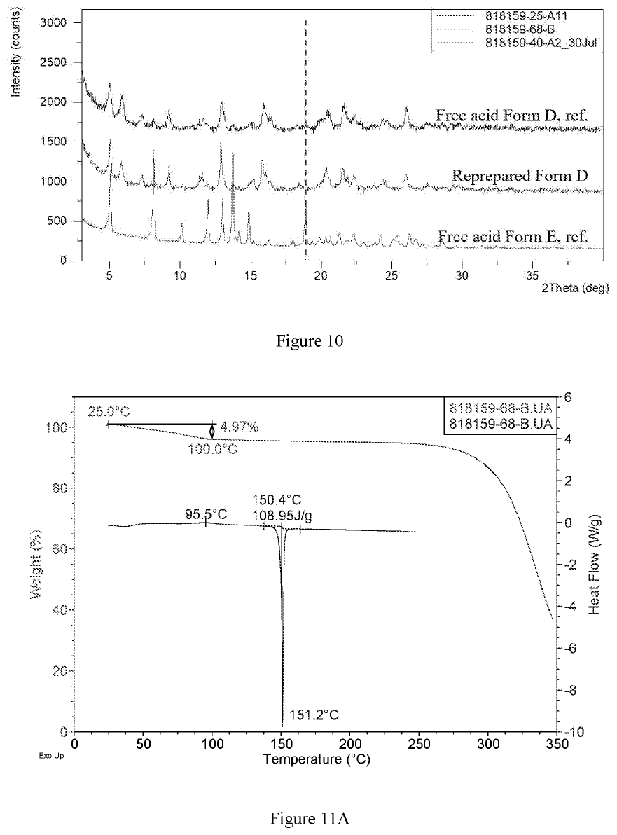

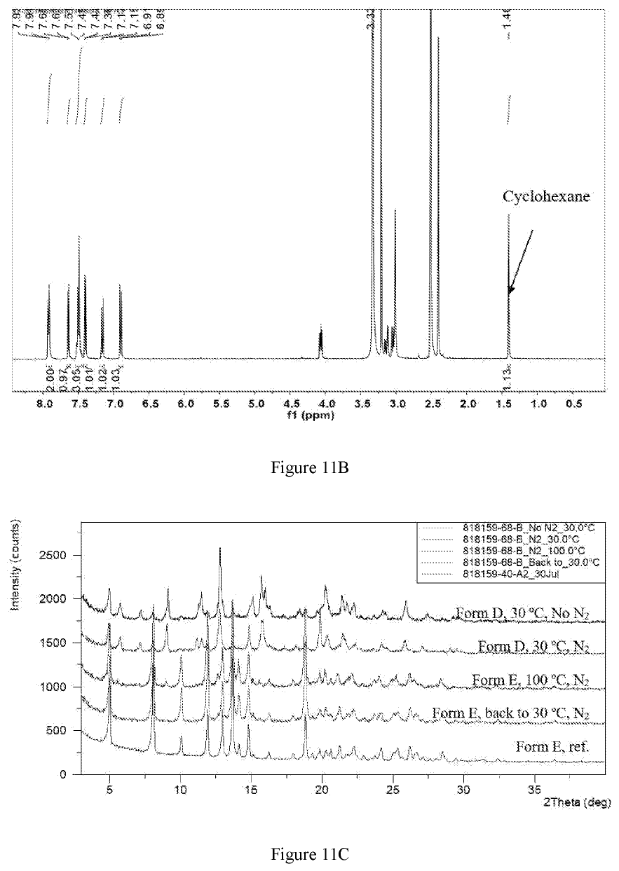

Another batch free acid Form D (818159-68-B) was re-prepared for form identification using the same method. As shown in FIG. 10, the re-prepared free acid Form D was a mixture of Form D and minor Form E. TGA result (FIG. 11A) showed a weight loss of 5.0% up to 100° C., and DSC result (FIG. 11A) showed one endothermic peak at 150.4° C. (one temperature) and one exotherm at 95.5° C. (peak temperature). ¹H NMR results (FIG. 11B) showed the molar ratio of cyclohexane to API was 0.09 (1.8 wt %). To investigate form origin, VT XRPD was conducted. As shown in FIG. 11C, 1) peak shift was observed after N₂ bleeding at 30° C.; 2) it converted to free acid Form E after heating to 100° C. under N₂ protection; 3) no form change was observed after cooling to 30° C. under N₂ protection. Combined characterization and VT results, it is speculated to be hydrate. Combined with VT XRPD results, the exotherm at 95.5° C. (peak temperature) is speculated to from transition, and the endothermic peak at 150.4° C. (one temperature) is consistent with that of Form E.

TABLE 23
XRPD of free acid Form D (818159-25-A11)

| Pos. | Height | FWHM | d-spacing | Rel. |
|---|---|---|---|---|
| [2θ] | [cts] | Left [°2θ] | [Å] | Int. [%] |
| 5.03 | 250.43 | 0.1535 | 17.56 | 100.00 |
| 5.90 | 178.97 | 0.2558 | 14.97 | 71.47 |
| 9.21 | 154.48 | 0.1535 | 9.61 | 61.69 |
| 11.76 | 54.20 | 0.6140 | 7.53 | 21.64 |
| 12.98 | 242.82 | 0.1791 | 6.82 | 96.96 |
| 15.04 | 49.29 | 0.4093 | 5.89 | 19.68 |
| 15.91 | 210.24 | 0.2558 | 5.57 | 83.95 |
| 20.47 | 149.51 | 0.2558 | 4.34 | 59.70 |
| 21.58 | 196.32 | 0.2047 | 4.12 | 78.39 |
| 22.38 | 110.96 | 0.3070 | 3.97 | 44.31 |
| 24.47 | 75.71 | 0.4093 | 3.64 | 30.23 |
| 26.08 | 178.89 | 0.2047 | 3.42 | 71.43 |

### 3.4 Crystalline Form E of Compound 2

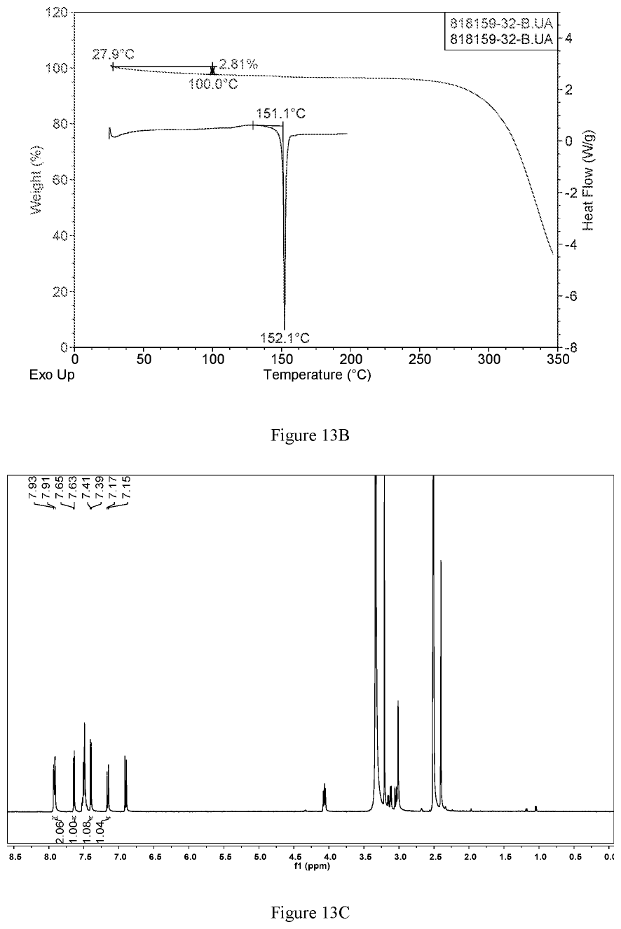

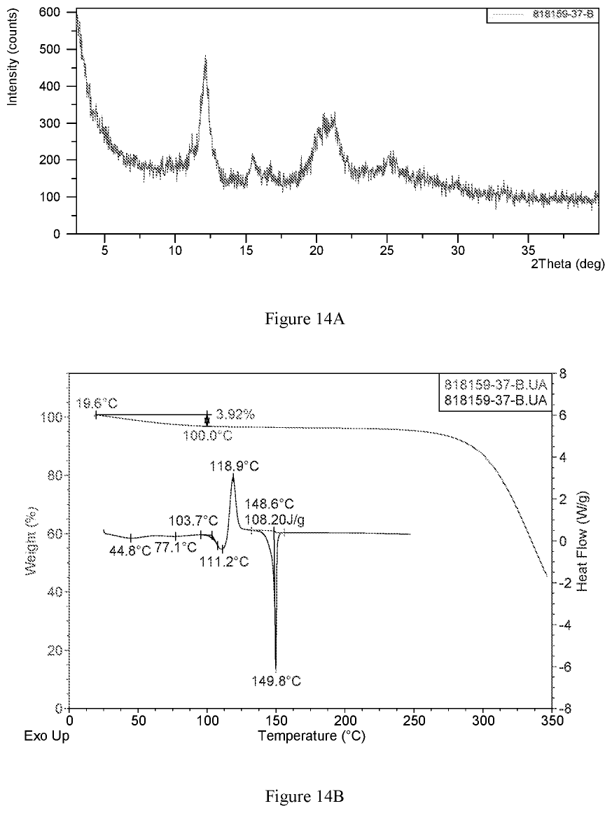

Free acid Form E was observed in few 50° C. slurry experiments. While pure free acid Form E (818159-40-A2_30Jul) was obtained via slurry competition experiment in MIBK at 50° C. XRPD is displayed in FIG. 12 and Table 24. Free acid Form E (818159-32-B) was prepared via slurry starting material in IPA at 50° C., as shown in FIG. 13A. After further comparison, minor Form B was observed in free acid Form E (818159-32-B). TGA result (FIG. 13B) showed a weight loss of 2.8% up to 100° C., and DSC result (FIG. 13B) showed one endothermic peak at 151.1° C. (onset temperature) which was speculated to be melting. ¹H NMR results (FIG. 13C) showed no residual IPA was observed. Considering low TGA weight loss and neat DSC, it is speculated to be anhydrate.

TABLE 24
XRPD of free acid Form E (818159-40-A2_30Jul)

| Pos. | Height | FWHM | d-spacing | Rel. |
|---|---|---|---|---|
| [2θ] | [cts] | Left [°2θ] | [Å] | Int. [%] |
| 5.06 | 2249.89 | 0.1023 | 17.46 | 62.44 |
| 8.14 | 3411.39 | 0.1023 | 10.86 | 94.68 |
| 10.12 | 699.92 | 0.1023 | 8.74 | 19.43 |
| 11.97 | 1578.24 | 0.1279 | 7.39 | 43.80 |
| 13.02 | 1675.07 | 0.1023 | 6.80 | 46.49 |
| 13.73 | 3603.18 | 0.1023 | 6.45 | 100.00 |
| 14.19 | 508.48 | 0.1023 | 6.24 | 14.11 |
| 14.87 | 1205.68 | 0.1023 | 5.96 | 33.46 |
| 16.32 | 247.25 | 0.1023 | 5.43 | 6.86 |
| 18.00 | 192.01 | 0.1023 | 4.93 | 5.33 |
| 18.89 | 1650.00 | 0.1279 | 4.70 | 45.79 |
| 19.36 | 217.86 | 0.1023 | 4.58 | 6.05 |
| 19.89 | 341.60 | 0.1023 | 4.46 | 9.48 |
| 20.31 | 313.74 | 0.1023 | 4.37 | 8.71 |
| 20.67 | 356.03 | 0.1023 | 4.30 | 9.88 |
| 21.30 | 523.24 | 0.1791 | 4.17 | 14.52 |
| 22.33 | 502.17 | 0.1791 | 3.98 | 13.94 |
| 23.01 | 146.35 | 0.1535 | 3.87 | 4.06 |
| 23.78 | 179.70 | 0.1023 | 3.74 | 4.99 |
| 24.23 | 462.77 | 0.1279 | 3.67 | 12.84 |
| 25.07 | 260.42 | 0.1535 | 3.55 | 7.23 |
| 25.40 | 428.60 | 0.1535 | 3.51 | 11.90 |
| 26.23 | 526.81 | 0.1791 | 3.40 | 14.62 |
| 26.74 | 307.98 | 0.2047 | 3.33 | 8.55 |
| 27.18 | 139.64 | 0.1535 | 3.28 | 3.88 |
| 27.68 | 88.63 | 0.1535 | 3.22 | 2.46 |
| 28.57 | 343.68 | 0.2047 | 3.12 | 9.54 |
| 29.51 | 111.78 | 0.1535 | 3.03 | 3.10 |
| 31.40 | 101.86 | 0.2047 | 2.85 | 2.83 |
| 32.47 | 109.68 | 0.1535 | 2.76 | 3.04 |
| 36.45 | 92.19 | 0.2047 | 2.47 | 2.56 |
| 39.05 | 26.13 | 0.3070 | 2.31 | 0.73 |

Peak in blue is the characteristic peak different from crystalline form B.

### 3.5 Crystalline Form F of Compound 2

Free acid Form F (818159-37-B) was obtained via evaporation in THF/H₂O (4:1, v/v) at RT, as shown in FIG. 14A and Table 25. Free acid Form F was also observed in residual solids from kinetic solubility starting from different salts. TGA result (FIG. 14B) showed a weight loss of 3.9% up to 100° C., and DSC result (FIG. 14B) showed four endothermic peaks at 44.8° C., 77.1° C., 111.2° C. and 149.8° C. (peak temperature) and one exotherm at 118.9° C. (peak temperature). ¹H NMR results (FIG. 14C) showed no residual THF was observed. To investigate form origin, VT XRPD was conducted. As shown in FIG. 14D, 1) no form change was observed after N₂ bleeding at 30° C.; 2) it converted to free acid Form E after heating to 130° C. under N₂ protection; 3) no form change was observed after cooling to 30° C. under N₂ protection. Combined characterization and preparation conditions, it is speculated to be hydrate. The endotherms before 100° C. are speculated to be dehydrate and the exotherm at 118.9° C. (peak temperature) is speculated to be form transition. The endothermic peak at 149.8° C. (peak temperature) is consistent with that of Form E.

TABLE 25
XRPD of free acid Form F (818159-37-B)

| Pos. | Height | FWHM | d-spacing | Rel. |
|---|---|---|---|---|
| [2θ] | [cts] | Left [°2θ] | [Å] | Int. [%] |
| 12.12 | 302.99 | 0.3070 | 7.30 | 100.00 |
| 15.50 | 72.25 | 0.5117 | 5.72 | 23.85 |
| 20.21 | 132.75 | 0.8187 | 4.39 | 43.81 |
| 21.23 | 154.02 | 0.6140 | 4.19 | 50.83 |

### 3.6 Crystalline Form G of Compound 2

Free acid Form G (818159-41-B) was obtained via evaporation in DMSO/MTBE at RT. XRPD and TGA/DSC results are displayed in FIG. 15A and Table 26, and FIG. 15B, respectively. TGA result showed a weight loss of 36.7% up to 150° C., and DSC result showed two endothermic peaks at 72.4° C. and 83.1° C. (peak temperature). ¹H NMR (FIG. 15C) was tested using CD₃OD as solvent and the results showed the molar ratio of DMSO/free acid was 1.7 (23.0 wt %). To further investigate form origin, heating experiment was conducted. As a result, melting was observed after heating to 110° C. and then cooling to ambient. Based on characterization and heating experiments results, free acid Form G is speculated to be DMSO solvate. The endotherms are speculated to be desolvation.

TABLE 26
XRPD of free acid Form G (818159-41-B)

| Pos. | Height | FWHM | d-spacing | Rel. |
|---|---|---|---|---|
| [2θ] | [cts] | Left [°2θ] | [Å] | Int. [%] |
| 4.95 | 564.55 | 0.1535 | 17.84 | 31.91 |
| 8.16 | 668.52 | 0.0768 | 10.84 | 37.79 |
| 9.67 | 1336.43 | 0.1023 | 9.14 | 75.54 |
| 11.60 | 1423.52 | 0.1023 | 7.63 | 80.47 |
| 11.83 | 543.62 | 0.1023 | 7.48 | 30.73 |
| 12.90 | 1769.08 | 0.1023 | 6.86 | 100.00 |
| 13.22 | 405.43 | 0.0768 | 6.70 | 22.92 |
| 13.69 | 911.34 | 0.1023 | 6.47 | 51.51 |
| 13.91 | 637.15 | 0.0768 | 6.37 | 36.02 |
| 14.43 | 703.50 | 0.1023 | 6.14 | 39.77 |
| 14.84 | 342.06 | 0.1791 | 5.97 | 19.34 |
| 16.15 | 357.74 | 0.1535 | 5.49 | 20.22 |
| 18.51 | 580.67 | 0.1023 | 4.79 | 32.82 |
| 18.93 | 786.03 | 0.1535 | 4.69 | 44.43 |
| 19.84 | 458.42 | 0.1535 | 4.47 | 25.91 |
| 20.47 | 772.43 | 0.1023 | 4.34 | 43.66 |
| 22.70 | 222.18 | 0.1279 | 3.92 | 12.56 |
| 25.28 | 247.65 | 0.2047 | 3.52 | 14.00 |
| 26.24 | 329.44 | 0.1535 | 3.40 | 18.62 |
| 26.81 | 266.57 | 0.2047 | 3.33 | 15.07 |
| 27.59 | 213.71 | 0.1535 | 3.23 | 12.08 |
| 28.60 | 161.94 | 0.3582 | 3.12 | 9.15 |
| 29.94 | 75.13 | 0.1535 | 2.98 | 4.25 |
| 35.09 | 119.77 | 0.2047 | 2.56 | 6.77 |
| 35.93 | 59.60 | 0.3070 | 2.50 | 3.37 |

### Example 4: Inter-Conversion Relationship Study

### 4.1 Slurry Competition of Anhydrates

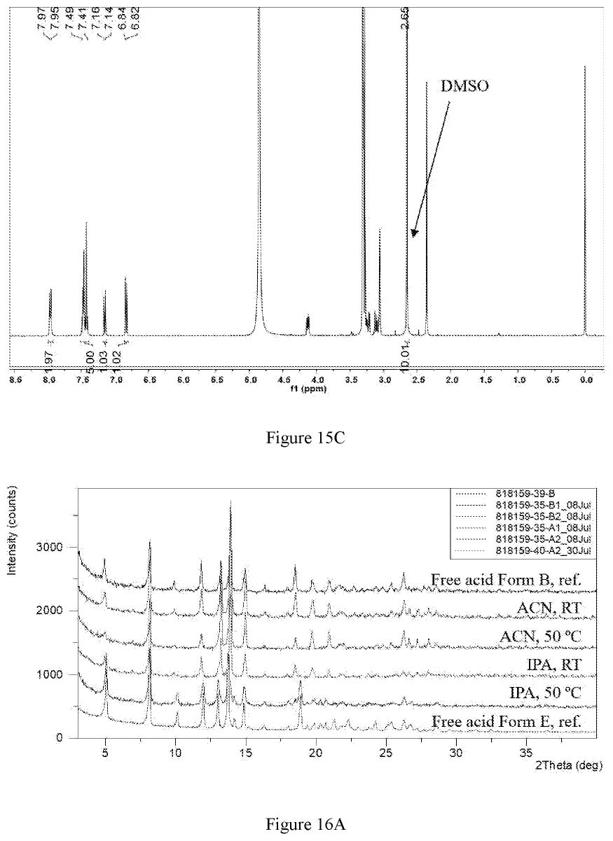

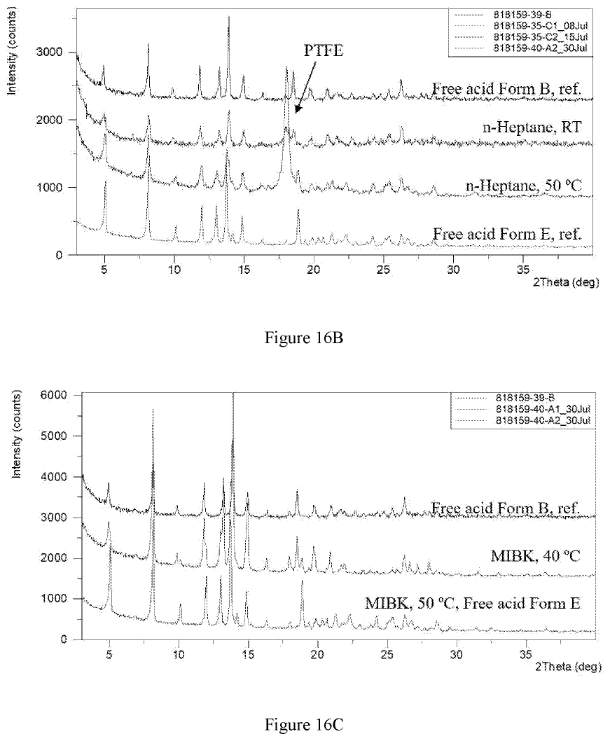

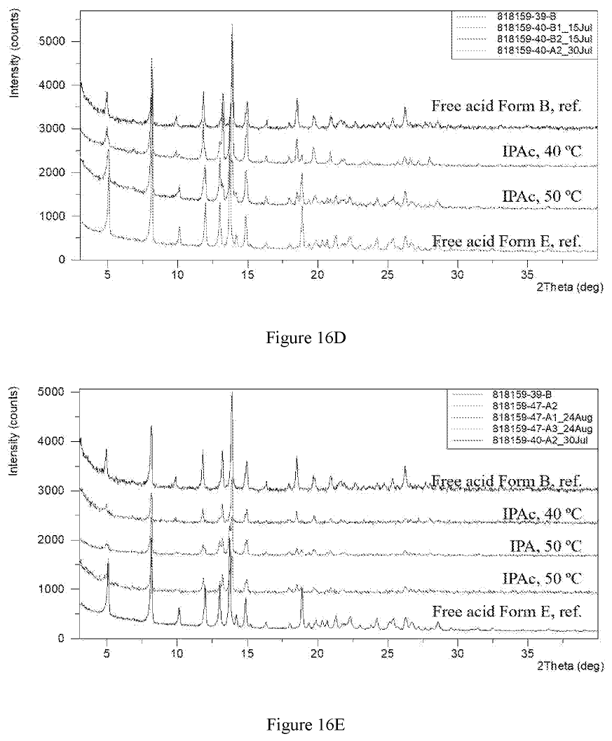

In order to investigate inter-conversion relationship among free acid anhydrates Form B/C/E, slurry competitive experiments were performed in IPA, ACN and n-heptane at RT and 50° C. Procedure: a mixture of corresponding forms with approximately equal mass was added into pre-saturated solutions and stirred at corresponding temperatures. The residual solids were extracted for XRPD analysis. The results were summarized in Table 27 and XRPD results were displayed in FIGS. 16A-16E. Based on the results, Form B was obtained in all tested solvents at RT and Form E was observed in IPA and n-heptane at 50° C. While, Form B was observed in ACN both at RT and 50° C., possibly due to solvent effect. To further investigate the transition temperature of Form B and Form E, slurry completion experiments were conducted in MIBK and IPAc at 40 and 50° C., resulting in Form B (major) at 40° C. and Form E (major) at 50° C. In following experiments (these experiments were conducted before Form D was identified), Form D material was added to IPA at 50° C. and IPAc at 40 and 50° C. The results showed Form B was observed at 40° C. and a mixture of Form B and E was observed at 50° C.

Above all, Form B and E are enantiotropic with Form B being the low temperature stable form. The transition temperature between Form B and E is between 40~50° C.

TABLE 27
Summary of slurry competition among anhydrate

| | Starting | | Temperature | |
|---|---|---|---|---|
| ID | Material | Solvent | (° C.) | Results |
| 818159-35-A1 | Form B/C/E | IPA | RT | Form B |
| 818159-35-A2 | | | 50 | Form E (Major) |
| 818159-35-B1 | | ACN | RT | Form B |
| 818159-35-B2 | | | 50 | Form B |
| 818159-35-C1 | | n-Heptane | RT | Form B (Major) |
| 818159-35-C2 | | | 50 | Form E (Major) |
| 818159-40-A1 | Form B/E | MIBK | 40 | Form B (Major) |
| 818159-40-A2 | | | 50 | Form E |
| 818159-40-B1 | | IPAc | 40 | Form B (Major) |
| 818159-40-B2 | | | 50 | Form E (Major) |
| 818159-47-A1 | Form B/D/E | IPA | 50 | Form B + E |
| 818159-47-A2 | | IPAc | 40 | Form B |
| 818159-47-A3 | | | 50 | Form B + E |

### 4.2 Slurry Competition of Anhydrates and Hydrates

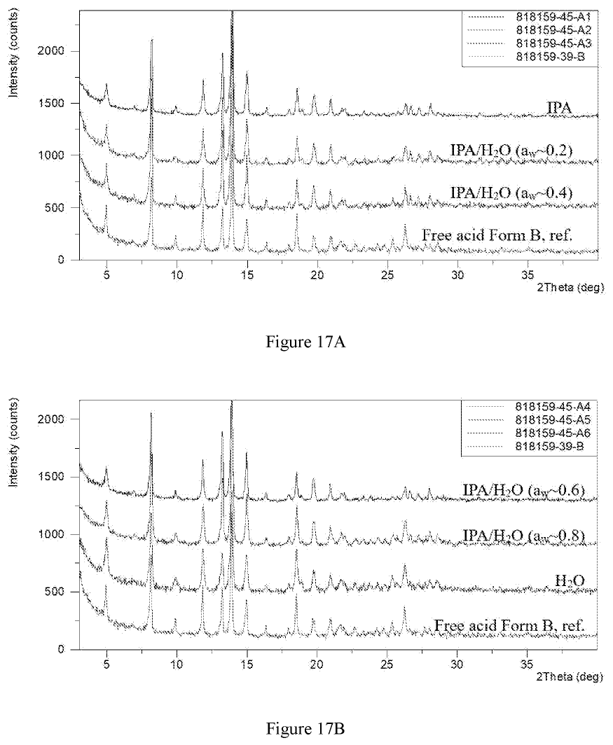

In order to investigate the inter-conversion relationship of free acid anhydrate Form B and hydrate Form D/F, the slurry competition was conducted at different water activity at RT in IPA/water. Procedure: a mixture of Form B/D/F with approximately equal mass was added into pre-saturated solutions and stirred at RT. The residual solids were extracted for XRPD analysis. The results were summarized in Table 28 and XRPD results were displayed in FIG. 17A and FIG. 17B. Based on the results, free acid Form B was obtained in all tested conditions.

TABLE 28
Summary of slurry competition at different water activities

| | Starting | | Temperature | |
|---|---|---|---|---|
| ID | Material | Solvent (v/v) | (° C.) | Results |
| 818159-45-A1 | Form | IPA | RT | Free acid |
| | B/D/F | | | Form B |
| 818159-45-A2 | | IPA/H₂O | | Free acid |
| | | (a_{w}~0.2, 0.982/0.018) | | Form B |
| 818159-45-A3 | | IPA/H₂O | | Free acid |
| | | (a_{w}~0.4, 0.956/0.044) | | Form B |
| 818159-45-A4 | | IPA/H₂O | | Free acid |
| | | (a_{w}~0.6, 0.92/0.08) | | Form B |
| 818159-45-A5 | | IPA/H₂O | | Free acid |
| | | (a_{w}~0.8, 0.847/0.153) | | Form B |
| 818159-45-A6 | | H₂O | | Free acid |
| | | | | Form B |

a_{w}: water activity

### Example 5: Salt Screening and Characterization

### 5.1 Salt Screening

Starting with free acid Form A (818159-01-A), 30 salt screening experiments were conducted using 10 bases and three solvent systems according to pKa (acidic, 3.36) and approximate solubility. Approximate 20 mg free acid and equimolar base was added in 0.5 mL solvent at RT. The solids were centrifuged for XRPD. As the results summarized in Table 29, a total of six salt hits were observed. All hits were characterized by TGA, DSC, ¹H NMR or HPLC/IC and the results are summarized in Table 30.

TABLE 29
Summary of salt screening

| | | | | C |
|---|---|---|---|---|
| | | A | B | Acetone/H₂O |
| # | Base* | EtOH | EtOAc | (19:1, v/v) |
| 0 | Blank | Free acid Form | Free acid Form | Free acid |
| | | B + E | B + E | Form B + E |
| 1 | NaOH | Free acid Form | Sodium salt | Amorphous |
| | | A | Form A | |
| 2 | KOH | K salt Form A + | K salt Form A | K salt Form |
| | | peaks | | B |
| 3 | L(+)- | Amorphous | Arginine salt | Oil |
| | Arginine | | Form A | |
| 4 | Ca (OH)₂ | Free acid Form | Free acid Form | Ca (OH)₂ |
| | | A + base + | A + base | |
| | | peaks | | |
| 5 | Mg (OH)₂ | Free acid Form | Free acid Form | Magnesium |
| | | A + base | A + base | salt Form A |
| 6 | Choline | Free acid Form | Free acid Form B | Low |
| | | A + B | | crystallinity |
| 7 | NH₃H₂O | Free acid Form | Free acid Form | Oil |
| | | A + B | B + peaks | |
| 8 | Meglumine | Gel | Gel (amorphous) | Gel |
| 9 | L-Lysine | Free acid Form | Free acid Form B | Low |
| | | A + B | | crystallinity |
| 10 | Tris | Gel | Tris salt Form A | Gel |

*Molar ratio of free acid/base is 1.0.

TABLE 30
Characterization summary of salt hits with from salt screening

| | | | | Stoichio- | HPLC |
|---|---|---|---|---|---|
| | | Weight | | metry | purity |
| | | Loss (%, | Endotherm | (Base/free | (area |
| Salt Form | ID | 150° C.) | (° C., peak) | acid) | %) |
| Sodium salt | 818159-03- | 4.5 (220) | 155.7*, 166.3^{#}, | 1.1 | 99.70 |
| Form A | B1 | | 201.8*, 288.7 | | |
| K salt | 818159-03- | 3.6 | 91.1, 135.8, | 0.9 | 99.93 |
| Form A | B2 | | 147.7 | | |
| Arginine salt | 818159-03- | 4.0 (180) | 186.9* | 1.0 | / |
| Form A | B3 | | | | |
| Magnesium | 818159-03- | 6.9 | 90.7, 114.9, | 0.8 | 99.72 |
| salt | | | | | |
| Form A | C5 | | 134.0, 154.2 | | |
| Tris salt | 818159-03- | 6.5 | 71.8, 76.6, | 1.0 | / |
| Form A | B10 | | 113.9, 213.1 | | |

*Endotherm (onset temperature);
^{#}Exothermic peak (peak temperature).

### 5.1.1 Na Salt Form A

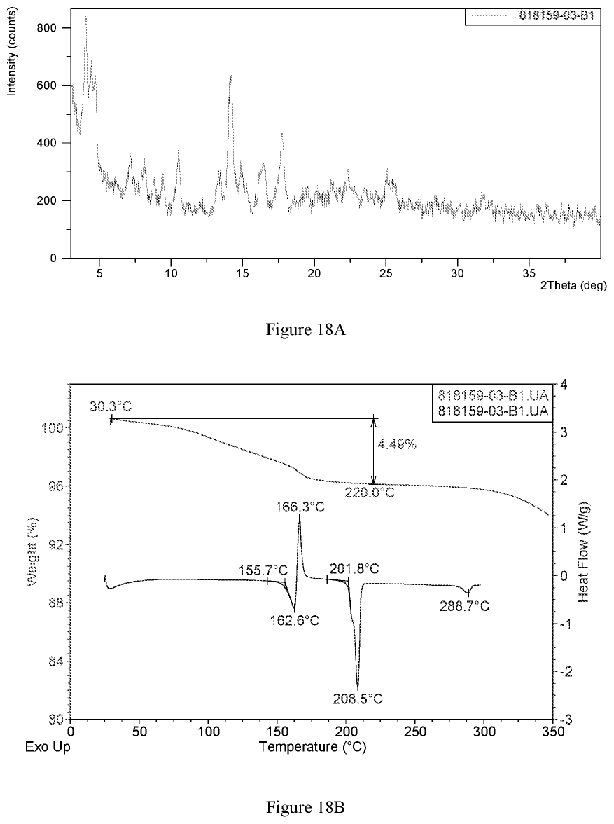

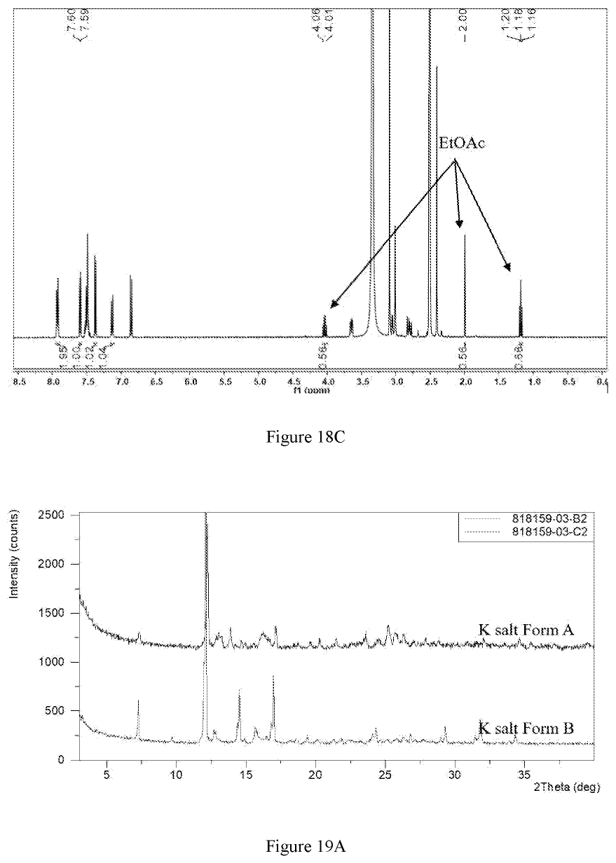

Na salt Form A (818159-03-B1) was prepared via slurry free acid Form A (818159-01-A) and NaOH with molar ratio of 1:1 (base/acid) in EtOAc at RT for 3 days, followed by centrifugation and vacuum drying at 50° C. XRPD and TGA/DSC results are displayed in FIG. 18A and FIG. 18B. TGA result showed a weight loss of 4.5% up to 220° C., and DSC result showed three endothermic peaks at 155.7, 201.8° C. (onset temperature) and 288.7° C. (peak temperature) and one exotherm at 166.3° C. (peak temperature). ¹H NMR (FIG. 18C) results showed the molar ratio of EtOAc/free acid was 0.19 (0.8 wt %). HPLC/IC showed the molar ratio of base/acid was 1.1.

### 5.1.2 K Salt Form A/B

K salt Form A/B (818159-03-B2/C2) was prepared via slurry free acid Form A (818159-01-A) and KOH with molar ratio of 1:1 (base/acid) in EtOAc and acetone/H₂O at RT for 3 days, followed by centrifugation and vacuum drying at 50° C., as displayed in FIG. 19A. For K salt Form A, TGA/DSC results are displayed in FIG. 19B. TGA result showed a weight loss of 3.6% up to 150° C., and DSC result showed three endothermic peaks at 91.1, 135.8 and 147.7° C. (peak temperature). 1H NMR (FIG. 19C) results showed no EtOAc was detected. HPLC/IC showed the molar ratio of base/acid was 1.1 for K salt Form A. The characterization of K salt Form B was not collected due to limited material.

### 5.1.3 Arginine Salt Form A

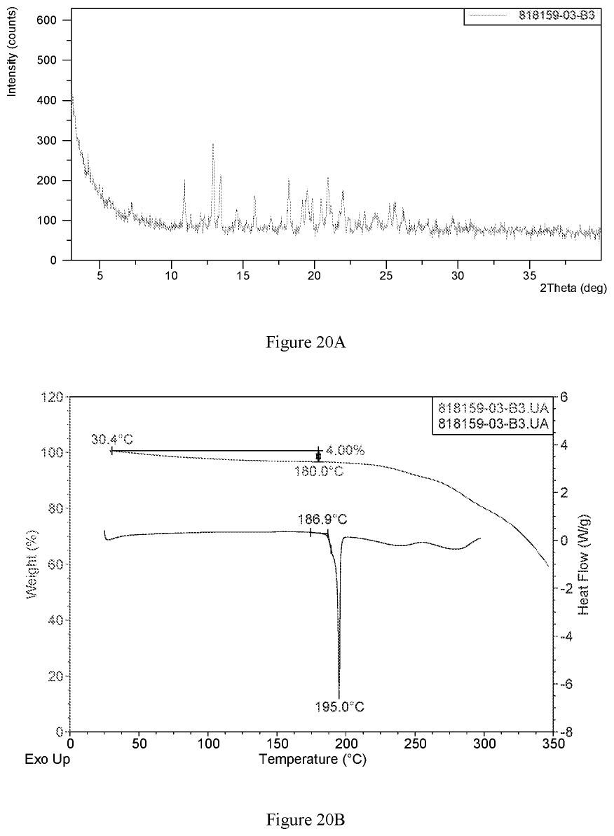

Arginine salt Form A (818159-03-B3) was prepared via slurry free acid Form A (818159-01-A) and arginine with molar ratio of 1:1 (base/acid) in EtOAc at RT for 3 days, followed by centrifugation and vacuum drying at 50° C. XRPD and TGA/DSC results are displayed in FIG. 20A and FIG. 20B. TGA result showed a weight loss of 4.0% up to 180° C., and DSC result showed one endothermic peak at 186.9° C. (onset temperature). ¹H NMR (FIG. 20C) results showed the molar ratio of arginine/free acid was 1.0 and the molar ratio of EtOAc/free acid was 0.12 (2.3 wt %).

### 5.1.4 Mg Salt Form A

Mg salt Form A (818159-03-C5) was prepared via slurry free acid Form A (818159-01-A) and MgOH with molar ratio of 1:1 (base/acid) in acetone at RT for 3 days, followed by centrifugation and vacuum drying at 50° C. XRPD and TGA/DSC results are displayed in FIG. 21A and FIG. 21B. TGA result showed a weight loss of 6.9% up to 150° C., and DSC result showed four endothermic peaks at 90.7, 114.9, 134.0 and 154.2° C. (peak temperature). ¹H NMR (FIG. 21C) results showed the molar ratio of acetone/free acid was 0.01 (0.2 wt %). HPLC/IC showed the molar ratio of base/acid was 0.8.

### 5.1.5 Tris Salt Form A

Tris salt Form A (818159-03-B10) was prepared via slurry free acid Form A (818159-01-A) and tris with molar ratio of 1:1 (base/acid) in EtOAc at RT for 3 days, followed by centrifugation and vacuum drying at 50° C. XRPD and TGA/DSC results are displayed in FIG. 22A and FIG. 22B. TGA result showed a weight loss of 6.5% up to 150° C., and DSC result showed four endothermic peaks at 71.8, 76.6, 113.9 and 213.1° C. (peak temperature). ¹H NMR (FIG. 22C) results showed the molar ratio of tris/API was 1.0 and the molar ratio of EtOAc/API was 0.26 (4.9 wt %).

### 5.2 Salt Leads Re-Preparation

Based on characterization results, it is speculated that arginine salt Form A is anhydrate and the other salts are hydrate or solvate. Considering the safety of salt co-former, dehydration temperature, Na salt Form A, K salt Form A and arginine salt Form A are selected as salt leads for re-preparation and evaluation. A new K salt (K salt Form C) was obtained during re-preparation, and the other salts were successfully re-prepared. The detailed preparation procedures and characterization results are summarized in Table 31 and Table 32.

TABLE 31
Re-preparation procedures of salt leads

| Salt Form | | Procedures |
|---|---|---|
| Na salt Form A | 1. | Mixture ~300.4 mg free acid (818159-01-A) |
| (818159-09-B) | | and 27.4 mg NaOH in 20-mL vial and add |
| | | 7 mL EtOAc to form suspension. |
| | 2. | Slurry at RT for six days followed by |
| | | filtration and vacuum drying at 50° C. for |
| | | 3.5 hrs. |
| K salt Form C | 1. | Mixture ~300.1 mg free acid (818159-01-A) |
| (818159-10-B) | | and 38.9 mg KOH in 20-mL vial and add |
| | | 7 mL EtOAc to form suspension. |
| | 2. | Slurry at RT for six days followed by |
| | | filtration and vacuum drying at 50° C. for |
| | | 3.5 hrs. |
| Arginine salt | 1. | Mixture ~299.5 mg free acid (818159-01-A) |
| Form A | | and 119.5 mg arginine in 20-mL vial and add |
| (818159-11-B) | | 7 mL EtOAc to form suspension. |
| | 2. | Slurry at RT for six days followed by |
| | | filtration and vacuum drying at 50° C. for |
| | | 3.5 hrs. |

TABLE 32
Characterization summary of salt leads

| | | Weight | | Stoichiometry |
|---|---|---|---|---|
| | | Loss (%, | Endotherm | (Base/free |
| Salt Form | ID | temp.) | (° C., peak) | acid) |
| Na salt Form A | 818159-09-B | 6.2 (220) | 157.4*, 166.5^{#}, | 0.9 |
| | | | 204.4 | |
| K salt Form C | 818159-10-B | 5.5 (200) | 108.1, 140.2, | 0.9 |
| | | | 161.3 | |
| Arginine salt | 818159-11-B | 1.3 (150) | 194.0* | 1.0 |
| Form A | | | | |

*onset temperature;
^{#}Exothermic peak.

### 5.2.1 Na Salt Form A

Na salt Form A (818159-09-B) was re-prepared, as XRPD results displayed in FIG. 24A, FIG. 23 and Table 33. A new form was observed in wet sample and converted to Na salt Form A. TGA/DSC results are displayed in FIG. 24B. TGA result showed a weight loss of 6.2% up to 220° C., and DSC result showed two endothermic peaks at 157.4° C. (onset temperature) and 204.0° C. (peak temperature) and one exotherm at 166.5° C. (peak temperature). ¹H NMR (FIG. 24C) results showed the molar ratio of EtOAc/free acid was 0.31 (5.8 wt %). HPLC/IC showed the molar ratio of base/acid was 0.9.

TABLE 33
XRPD of Na salt Form A (818159-09-B)

| Pos. [°2θ] | Height [cts] | FWHM Left [°2θ] | d-spacing [Å] | Rel. Int. [%] |
|---|---|---|---|---|
| 4.09 | 253.11 | 0.2047 | 21.62 | 100.00 |
| 4.62 | 169.81 | 0.3070 | 19.13 | 67.09 |
| 14.21 | 202.87 | 0.3070 | 6.23 | 80.15 |
| 16.43 | 98.11 | 0.5117 | 5.39 | 38.76 |
| 17.72 | 103.49 | 0.3070 | 5.00 | 40.89 |

### 5.2.2 K Salt Form C

A new K salt, assigned as K salt Form C (818159-10-B) was observed during re-preparation, as displayed in FIG. 26A, FIG. 25 and Table 34. TGA/DSC results are displayed in FIG. 26B. TGA result showed a weight loss of 5.5% up to 200° C., and DSC result showed three endothermic peaks at 108.1, 140.2 and 161.3° C. (peak temperature). ¹H NMR (FIG. 26C) results showed no EtOAc was detected. HPLC/IC showed the molar ratio of base/acid was 0.9.

TABLE 34
XRPD of K salt Form C (818159-10-B)

| Pos. [°2θ] | Height [cts] | FWHM Left [°2θ] | d-spacing [Å] | Rel. Int. [%] |
|---|---|---|---|---|
| 3.92 | 157.22 | 0.1535 | 22.53 | 32.29 |
| 6.90 | 74.02 | 0.3070 | 12.81 | 15.20 |
| 7.79 | 144.16 | 0.1279 | 11.35 | 29.60 |
| 12.91 | 486.96 | 0.1279 | 6.86 | 100.00 |
| 14.12 | 203.02 | 0.1535 | 6.27 | 41.69 |
| 14.96 | 337.98 | 0.1535 | 5.92 | 69.41 |
| 16.05 | 70.67 | 0.3070 | 5.52 | 14.51 |
| 16.83 | 67.03 | 0.3070 | 5.27 | 13.76 |
| 18.08 | 166.61 | 0.2558 | 4.91 | 34.21 |
| 19.11 | 91.29 | 0.5117 | 4.65 | 18.75 |
| 20.68 | 199.20 | 0.1791 | 4.30 | 40.91 |
| 21.23 | 259.04 | 0.1279 | 4.19 | 53.20 |
| 22.48 | 184.84 | 0.1023 | 3.96 | 37.96 |
| 23.28 | 114.92 | 0.2047 | 3.82 | 23.60 |
| 24.70 | 161.50 | 0.1279 | 3.60 | 33.16 |
| 25.17 | 231.59 | 0.1791 | 3.54 | 47.56 |
| 25.81 | 162.36 | 0.2047 | 3.45 | 33.34 |
| 26.46 | 201.70 | 0.1535 | 3.37 | 41.42 |
| 30.84 | 80.36 | 0.3070 | 2.90 | 16.50 |

### 5.2.3 Arginine Salt Form A

Arginine salt Form A (818159-11-B) was re-prepared as shown in FIG. 28A, FIG. 27 and Table 35. TGA result (FIG. 28B) showed a weight loss of 1.3% up to 150° C., and DSC result (FIG. 28B) showed one endothermic peak at 194.0° C. (onset temperature). ¹H NMR (FIG. 28C) results showed the molar ratio of arginine/free acid was 1.0 and no EtOAc was detected.

TABLE 35
XRPD of Arginine salt Form A (818159-11-B)

| Pos. [°2θ] | Height [cts] | FWHM Left [°2θ] | d-spacing [Å] | Rel. Int. [%] |
|---|---|---|---|---|
| 7.25 | 96.76 | 0.1535 | 12.19 | 11.98 |
| 10.91 | 268.53 | 0.1023 | 8.11 | 33.25 |
| 11.34 | 105.61 | 0.1535 | 7.80 | 13.08 |
| 12.93 | 807.61 | 0.1023 | 6.85 | 100.00 |
| 13.43 | 486.31 | 0.0768 | 6.60 | 60.22 |
| 14.57 | 184.96 | 0.1023 | 6.08 | 22.90 |
| 15.83 | 325.96 | 0.1279 | 5.60 | 40.36 |
| 16.94 | 132.95 | 0.1023 | 5.23 | 16.46 |
| 18.21 | 432.06 | 0.1279 | 4.87 | 53.50 |
| 19.14 | 269.13 | 0.1023 | 4.64 | 33.32 |
| 19.49 | 337.40 | 0.1791 | 4.55 | 41.78 |
| 19.84 | 264.71 | 0.1023 | 4.48 | 32.78 |
| 20.45 | 253.40 | 0.1279 | 4.34 | 31.38 |
| 20.93 | 554.07 | 0.1023 | 4.24 | 68.61 |
| 21.15 | 268.54 | 0.1023 | 4.20 | 33.25 |
| 22.02 | 303.18 | 0.1279 | 4.04 | 37.54 |
| 23.46 | 111.06 | 0.1535 | 3.79 | 13.75 |
| 24.10 | 112.79 | 0.3070 | 3.69 | 13.97 |
| 25.22 | 173.22 | 0.1535 | 3.53 | 21.45 |
| 25.63 | 196.63 | 0.1535 | 3.48 | 24.35 |
| 26.15 | 193.41 | 0.1791 | 3.41 | 23.95 |
| 27.24 | 115.08 | 0.1535 | 3.27 | 14.25 |
| 27.81 | 79.92 | 0.2047 | 3.21 | 9.90 |
| 29.67 | 70.23 | 0.3070 | 3.01 | 8.70 |
| 30.90 | 58.60 | 0.4093 | 2.89 | 7.26 |
| 33.07 | 64.32 | 0.3070 | 2.71 | 7.96 |

### Example 6: Evaluation

Based on salt screening and characterization results, Na salt Form A, K salt Form C and arginine salt Form A were selected for evaluation using free acid Form B as control, including kinetic solubility, hygroscopicity and solid stability.

### 6.1 Kinetic Solubility Evaluation

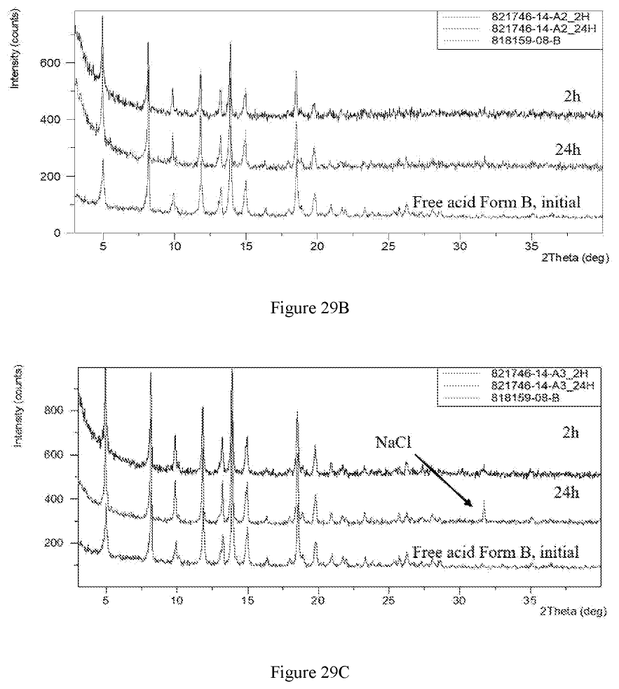

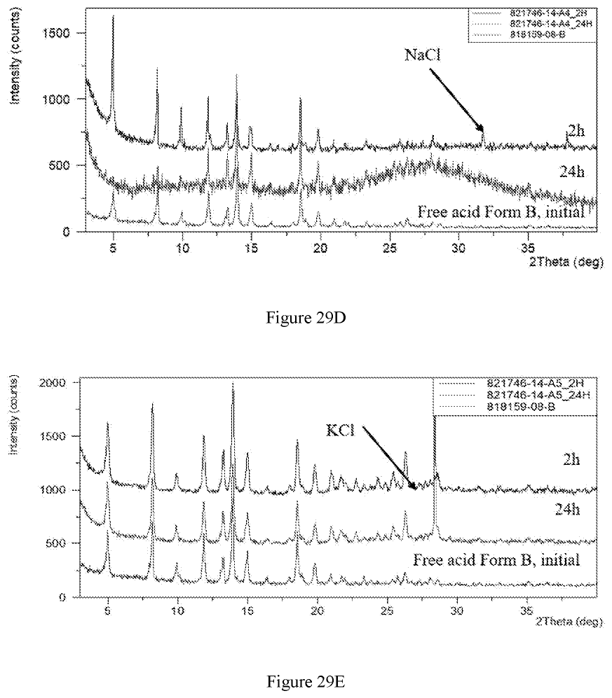

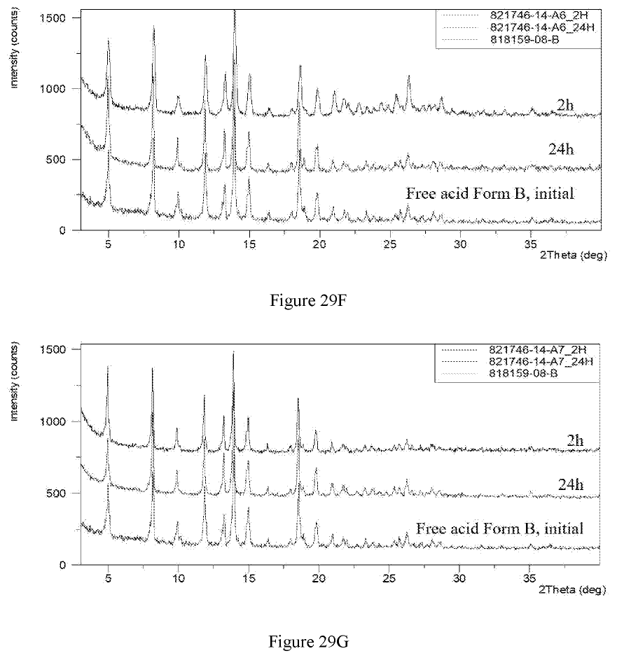

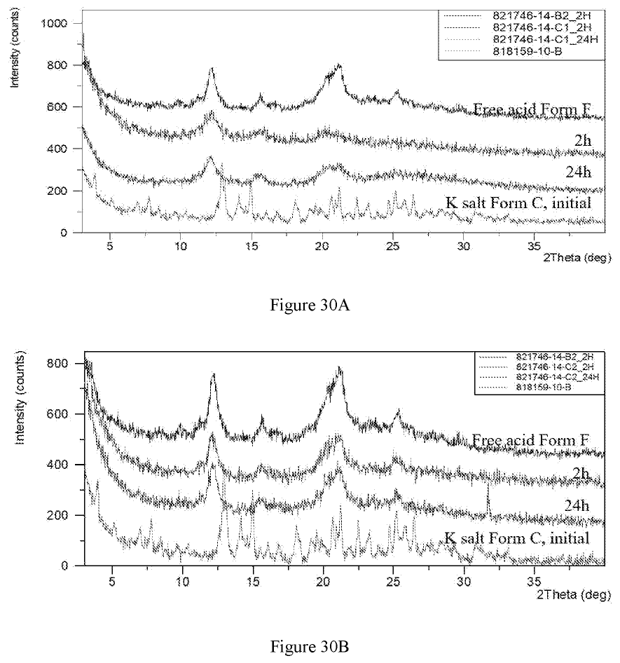

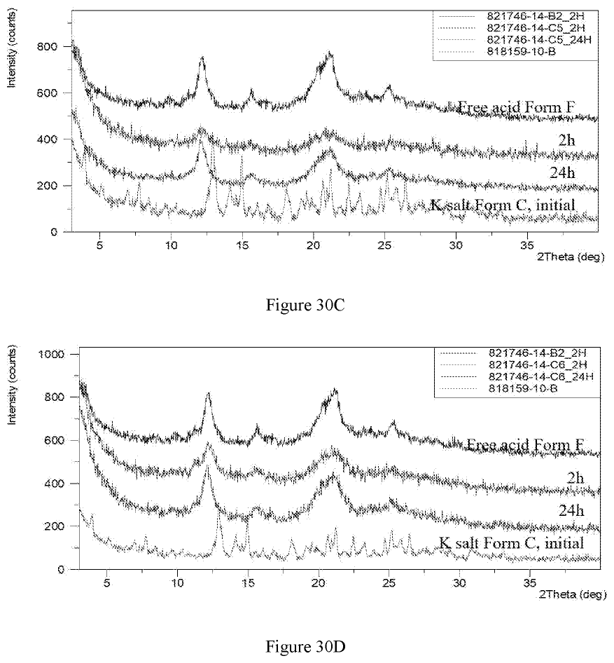

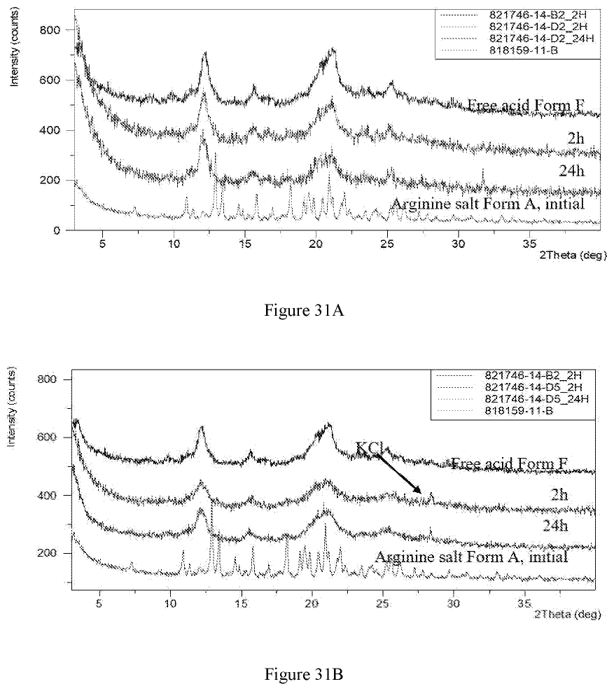

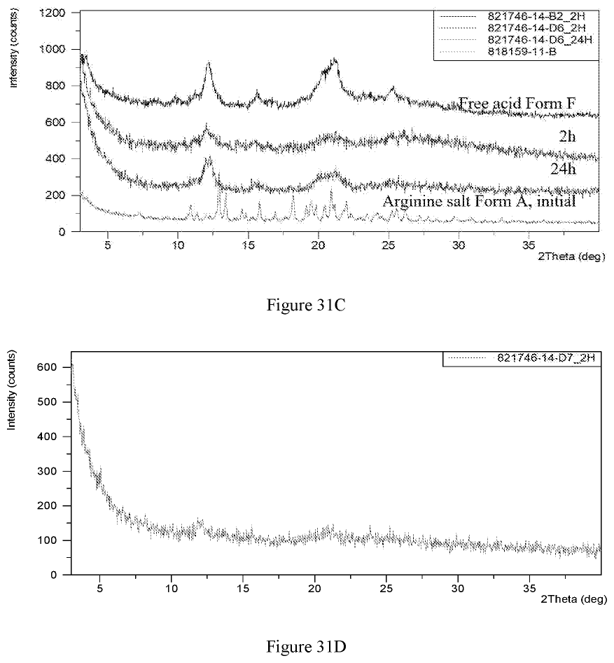

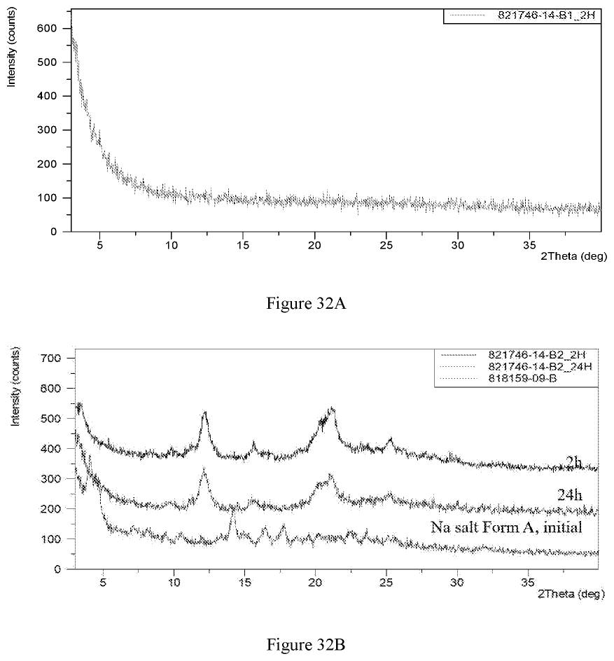

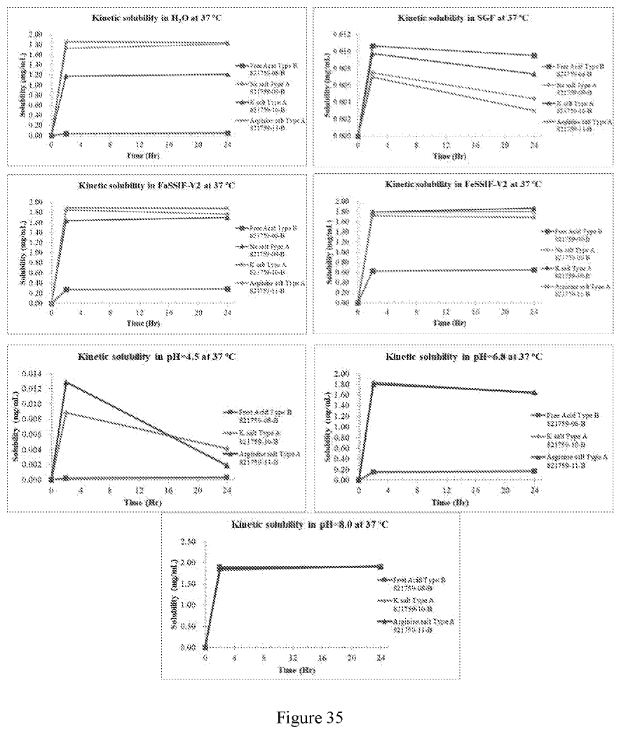

Kinetic solubility profiles were measured in water, SGF, FaSSIF-V2, FeSSIF-V2 and four pH buffers (pH 1.2, 4.5, 6.8 and 8.0 buffers) at 37° C. with solid loading of ~2 mg/mL (calculated as free acid). Na salt Form A was only measured in water, SGF, FaSSIF-V2 and FeSSIF-V2. In detail, salt solids were suspended into media and the suspensions were agitated on a rolling incubator. Sample at 2 and 24 hours, respectively. Supernatant was extracted via centrifugation before filtration through a 0.45 μm PTFE membrane, and used for solubility and pH measurement. Residual solids were collected for XRPD characterization (FIG. 29-FIG. 32). The results are summarized in Table 36, and the kinetic solubility profiles are displayed in FIG. 35. The results showed 1) Na salt Form A, K salt Form C and arginine salt Form A exhibited comparable solubility in water and three bio-relevant medias, with improved solubility compared with free acid Form B; 2) K salt Form C and arginine salt Form A exhibit comparable solubility in pH 4.5 and 6.8 buffers with improved solubility compared with free acid Form B; 3) no form change was observed after solubility test for free acid Form B, while, form change to free acid Form F or amorphous after solubility test for Na salt Form A, K salt Form C and arginine salt Form A.

TABLE 36
Kinetic solubility results summary at 37° C.

| | | 2 hours | | | 24 hours | | |
|---|---|---|---|---|---|---|---|
| Starting | | | | Form | | | Form |
| Material | Solvent | S | pH | change | S | pH | change |
| Free acid Form | H₂O | 0.036 | 8.3 | No | 0.051 | 6.5 | No |
| B | SGF | 0.011 | 1.9 | No | 0.0096 | 1.9 | No |
| (818159-08-B) | FaSSIF-V2 | 0.28 | 6.6 | No | 0.29 | 6.7 | No |
| | FeSSIF-V2 | 0.63 | 5.8 | No | 0.65 | 5.9 | No |
| | pH = 1.2 (KCl) | <LOD | 1.5 | No | <LOD | 1.5 | No |
| | pH = 4.5 (KAc) | 0.0003 | 4.8 | No | 0.0004 | 4.8 | No |
| | pH = 6.8* | 0.15 | 7.0 | No | 0.17 | 7.0 | No |
| | pH = 8.0* | 1.9 | 7.9 | NA | 1.9 | 8.0 | NA |
| K salt Form C | H₂O | 1.2 | 6.7 | Form F | 1.2 | 7.1 | Form F |
| (818159-10-B) | SGF | 0.0099 | 1.9 | Form F | 0.0075 | 2.1 | Form F |
| | FaSSIF-V2 | 1.9 | 6.5 | NA | 1.9 | 6.7 | NA |
| | FeSSIF-V2 | 1.8 | 5.8 | NA | 1.9 | 6.0 | NA |
| | pH = 1.2 (KCl) | <LOD | 1.4 | Form F | <LOD | 1.6 | Form F |
| | pH = 4.5 (KAc) | 0.089 | 4.8 | Form F | 0.0042 | 5.0 | Form F |
| | pH = 6.8* | 1.8 | 7.0 | NA | 1.6 | 7.2 | NA |
| | pH = 8.0* | 1.8 | 8.1 | NA | 1.9 | 8.3 | NA |
| Arginine salt | H₂O | 1.9 | 7.4 | NA | 1.8 | 7.9 | NA |
| Form A | SGF | 0.0071 | 2.0 | Form F | 0.0030 | 2.3 | Form F |
| (818159-11-B) | FaSSIF-V2 | 1.9 | 6.7 | NA | 1.8 | 7.0 | NA |
| | FeSSIF-V2 | 1.8 | 5.8 | NA | 1.8 | 6.1 | NA |
| | pH = 1.2 (KCl) | <LOD | 1.4 | Form F | <LOD | 1.7 | Form F |
| | pH = 4.5 (KAc) | 0.013 | 4.8 | Form F | 0.0020 | 5.1 | Form F |
| | pH = 6.8* | 1.8 | 7.0 | amorphous | 1.6 | 7.3 | NA |
| | pH = 8.0* | 1.9 | 8.2 | NA | 1.9 | 8.4 | NA |
| Sodium salt | H₂O | 1.7 | 6.6 | amorphous | 1.8 | 8.1 | N/A |
| Form A | SGF | 0.0076 | 2.0 | Form F | 0.0046 | 2.1 | Form F |
| (818159-09-B) | FaSSIF-V2 | 1.6 | 6.7 | NA | 1.7 | 6.9 | NA |
| | FeSSIF-V2 | 1.7 | 5.8 | NA | 1.7 | 6.0 | NA |

S: solubility (mg/mL);
LOD: 0.04 μg/mL;
N/A: not available due to limited solids;
*K₂HPO₃ + KH₂PO₃;
Form F: Free acid FormF.
All samples were suspension during solubility test.

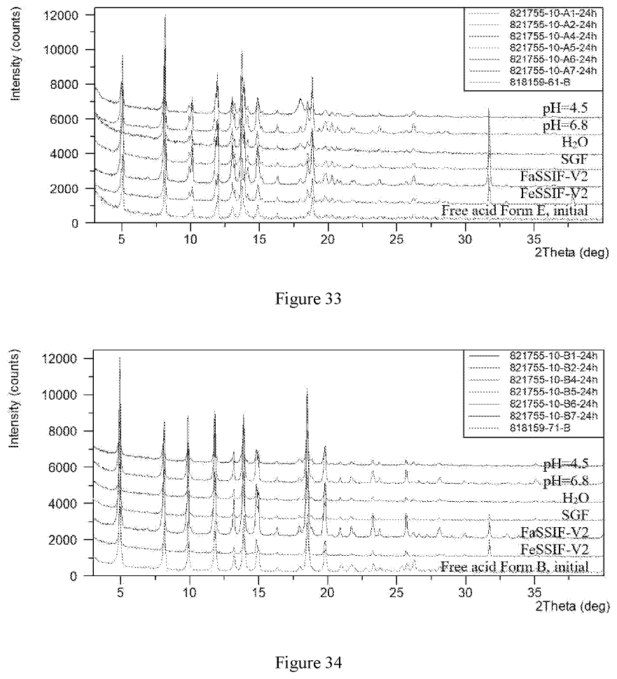

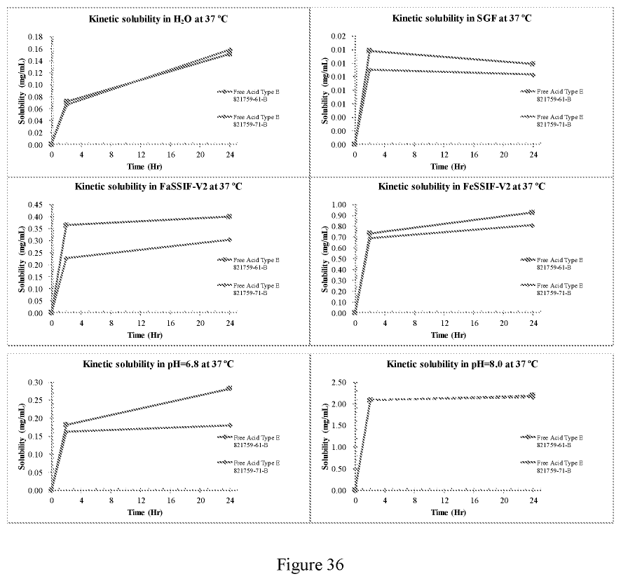

Based on characterization of different free acid forms and slurry competition results, free acid Form B and Form E are anhydrates and enantiotropic with Form B being the low temperature stable form. The transition temperature between Form B and E is between 40~50° C. To evaluate the differences on solubility, solubility tests were conducted in water, SGF, FaSSIF-V2, FeSSIF-V2 and four pH buffers (pH 1.2, 4.5, 6.8 and 8.0 buffers) at 37° C. with solid loading of ~2 mg/mL. In detail, salt solids were suspended into media and the suspensions were agitated on a rolling incubator. Sample at 2 and 24 hours, respectively. Supernatant was extracted via centrifugation before filtration through a 0.45 μm PTFE membrane, and used for solubility and pH measurement. Residual solids were collected for XRPD characterization (FIGS. 33 and 34). The results are summarized in Table 37, and the kinetic solubility profiles are displayed in FIG. 36. The results showed 1) free acid Form B and E showed comparable solubility in water and pH 8.0 (almost clear); 2) free acid Form E showed slightly higher solubility than Form B in the other medias; 3) no form change was observed after solubility tests.

TABLE 37
Kinetic solubility of free acid Form B and Form E at 37° C.

| | Material | | | | | | | | | |
|---|---|---|---|---|---|---|---|---|---|---|
| | Free acid Form E | | | | | Free acid Form B | | | | |
| | (818159-61-B) | | | | | (818159-71-B) | | | | |
| | | | 24 hours | | | | | 24 hours | | |
| | 2 hours | | | | Form | 2 hours | | | | Form |
| Solvent | S | pH | S | pH | change | S | pH | S | pH | change |
| H₂O | 0.072 | 8.7 | 0.15 | 8.4 | No | 0.066 | 8.6 | 0.16 | 8.4 | No |
| SGF | 0.014 | 1.8 | 0.012 | 1.8 | No | 0.011 | 1.8 | 0.010 | 1.8 | No |
| FaSSIF-V2 | 0.37 | 6.5 | 0.40 | 6.5 | No | 0.23 | 6.6 | 0.30 | 6.5 | No |
| FeSSIF-V2 | 0.73 | 5.8 | 0.93 | 5.8 | No | 0.69 | 5.8 | 0.81 | 5.8 | No |
| pH = 4.5 | <LOD | 4.6 | <LOD | 4.5 | No | <LOD | 4.6 | <LOD | 4.6 | No |
| (KAc) | | | | | | | | | | |
| pH = 6.8* | 0.18 | 6.7 | 0.28 | 6.7 | No | 0.16 | 6.7 | 0.18 | 6.7 | No |
| pH = 8.0* | 2.1 | 7.7 | 2.2 | 7.7 | N/A | 2.1 | 7.7 | 2.2 | 7.7 | N/A |

S: solubility (mg/mL);
LOD: 0.5~0.6 μg/mL;
N/A: not available due to clear solution.
*K₂HPO₃ + KH₂PO₃

### 6.2 Hygroscopicity Evaluation

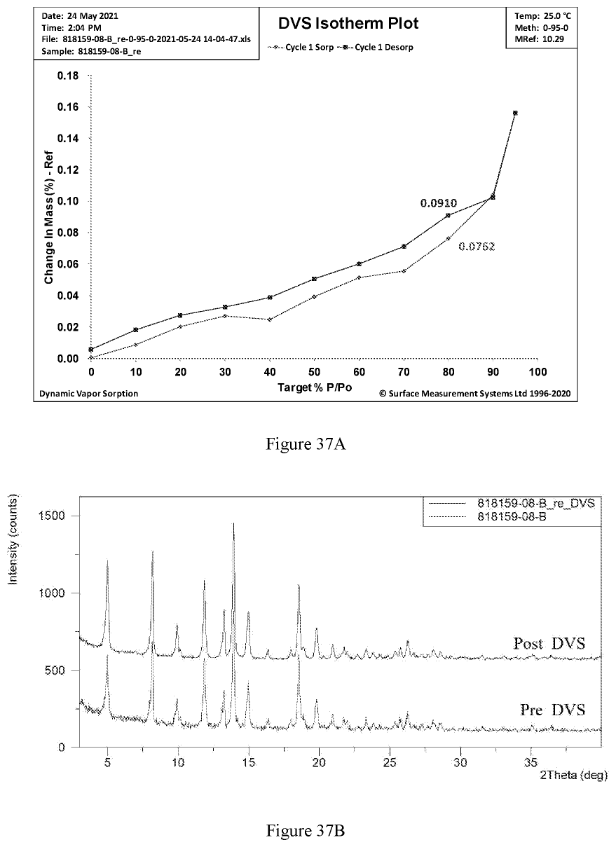

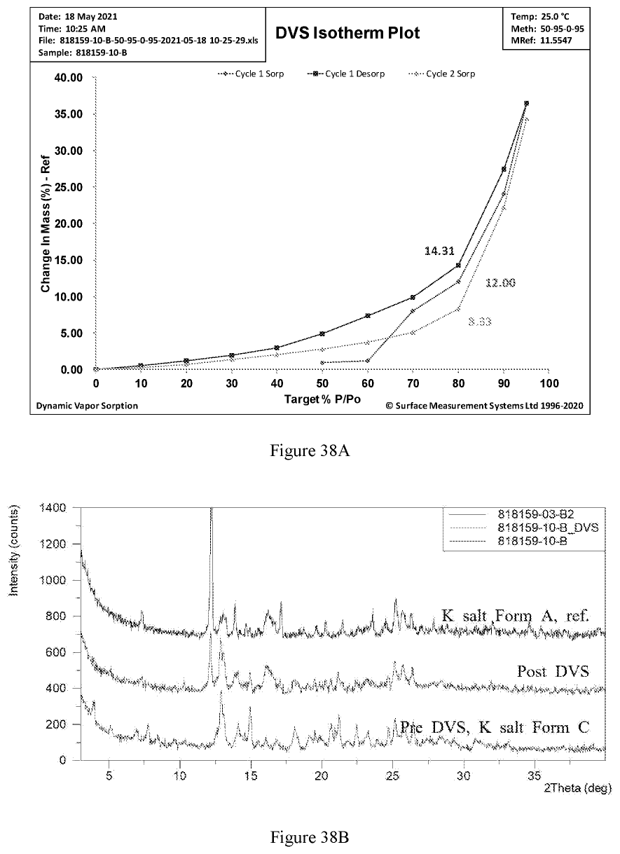

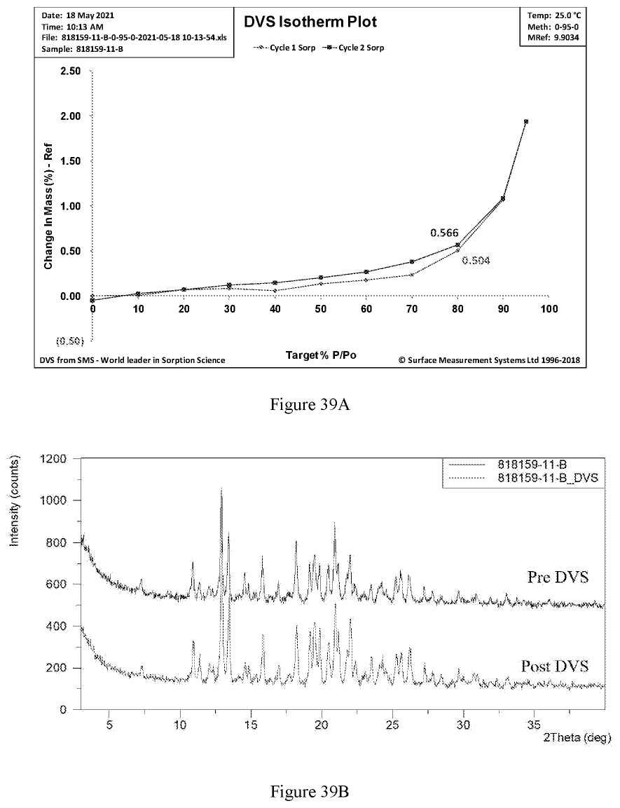

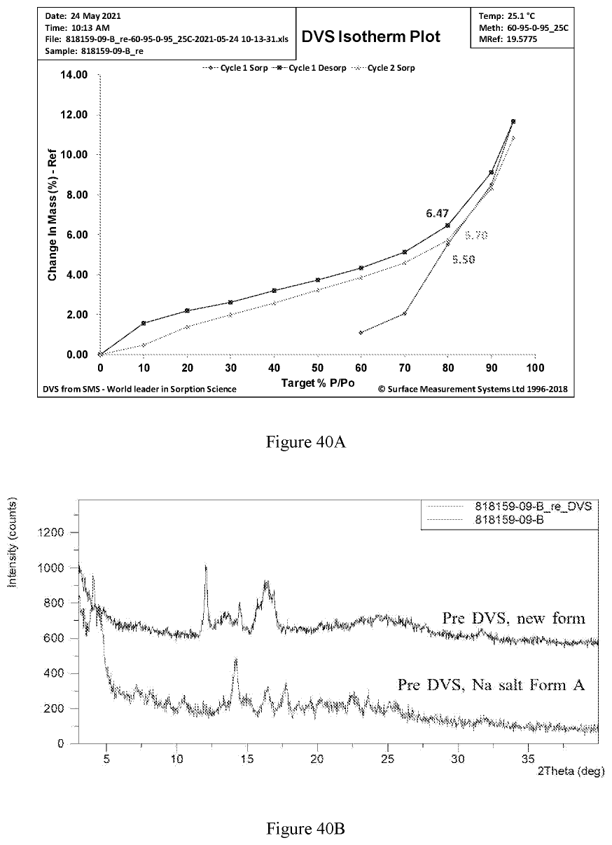

DVS isotherm plot of free acid Form B, Na salt Form A, K salt Form C and arginine salt Form A was collected at 25° C. to investigate the solid form stability as a function of humidity (0% RH, 50% RH and 60% RH). DVS plot and XRPD after DVS tests are displayed in FIGS. 37-40. A water uptake of 0.07% and 0.5% up to 80% RH for free acid Form B and arginine salt Form A, respectively, indicating that free acid Form B is non-hygroscopic and arginine salt Form A is slightly hygroscopicity. No form change was observed for free acid Form B and arginine salt Form A. A water uptake of 5.5% and 12.0% up to 80% RH for Na salt Form A and K salt Form C and form change was observed after DVS tests.

### 6.3 Solid Stability Evaluation

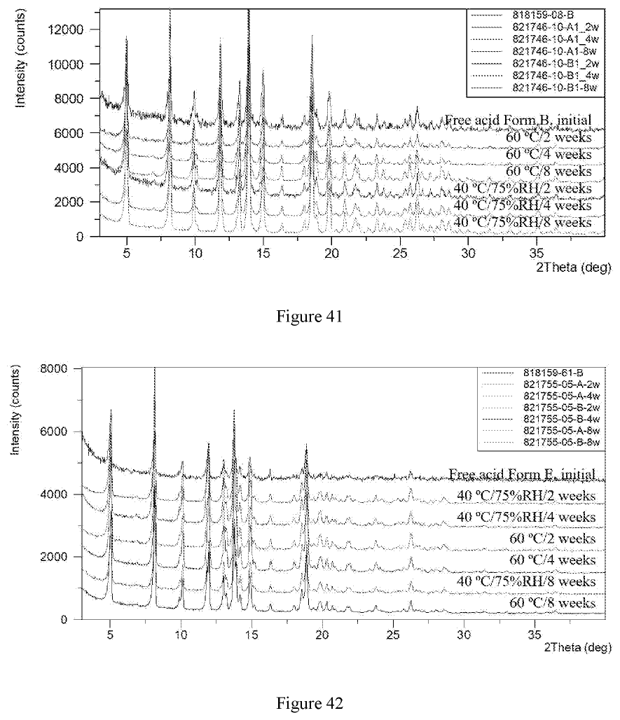

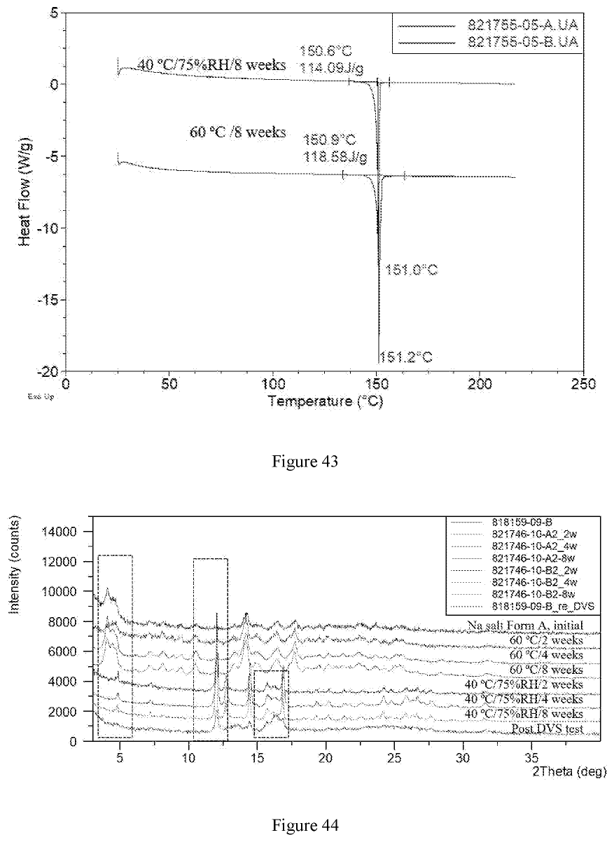

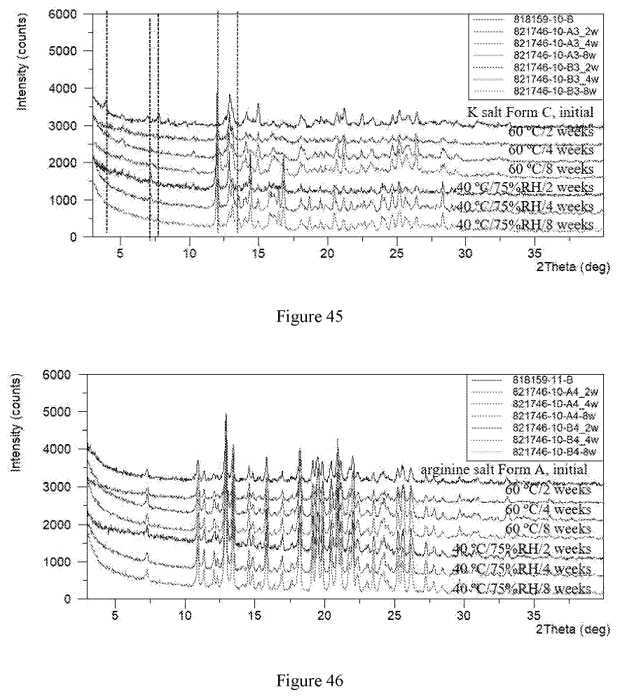

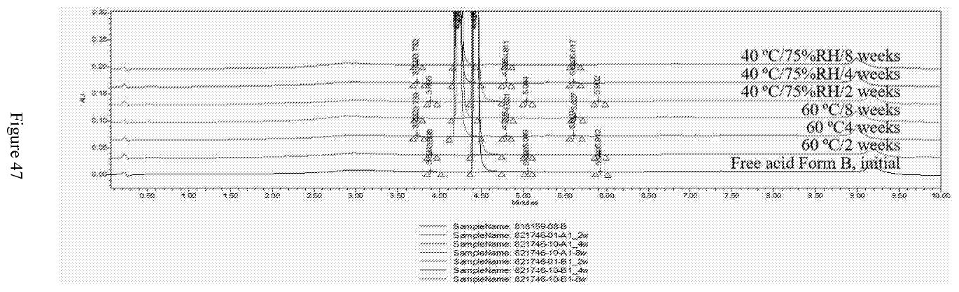

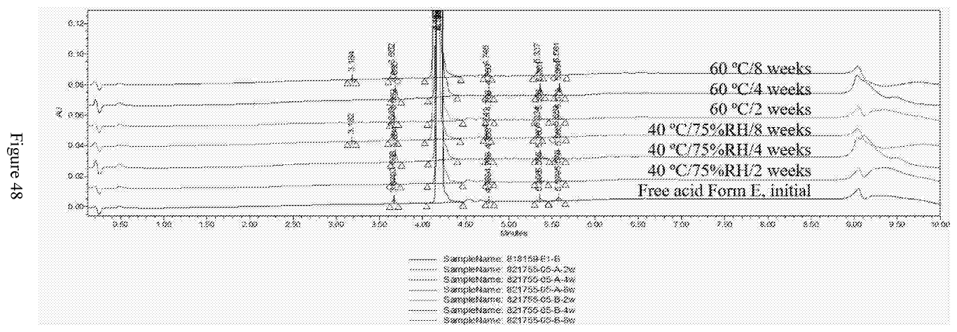

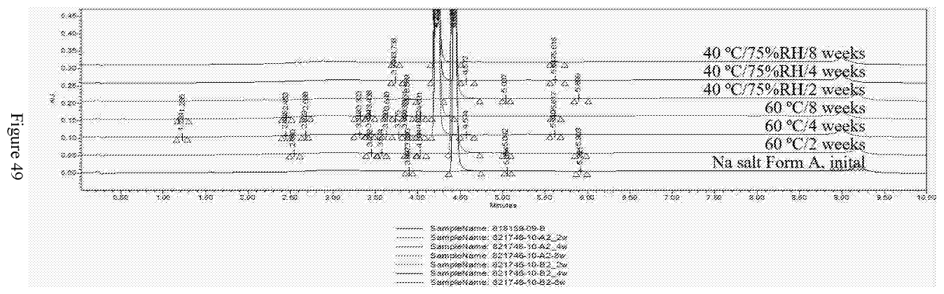

To understand solid stability, free acid Form B (Table 39)/E (Table 40), Na salt Form A (Table 41), K salt Form C (Table 42) and arginine salt Form A (Table 43) were evaluated for physical and chemical stability under 60° C./close and 40° C./75% RH/open for 2/4/8 weeks. The results are summarized in Table 38. XRPD results are displayed in FIG. 41, FIG. 42, FIG. 44, FIG. 45 and FIG. 46. The Form E samples after solid stability were tested by DSC (FIG. 43), and no significant difference was observed. HPLC results are displayed in FIGS. 47-51. No form or purity decrease was observed for free acid Form B/E and arginine salt Form A. No purity decrease but form change was observed for K salt Form C after storage under tested conditions. Slightly purity decrease and form change was observed for Na salt Form A after storage at 60° C. for 8 weeks.

TABLE 38
Solid stability evaluation summary

| | | | | HPLC | |
|---|---|---|---|---|---|
| | | | | Reults | |
| Form | | | Purity | of initial | |
| (ID) | Condition | | (area %) | (%) | Form |
| Free acid | Initial | | 99.47 | — | — |
| Form B* | 60° C./close | 2 weeks | 99.49 | 100.02 | Free acid |
| (818159- | | 4 weeks | 99.47 | 100.00 | Form B |
| 08-B) | | 8 weeks | 99.51 | 100.04 | |
| | 40° C./75% | 2 weeks | 99.50 | 100.03 | |
| | RH/open | 4 weeks | 99.47 | 100.00 | |
| | | 8 weeks | 99.52 | 100.05 | |
| Free acid | Initial | | 99.01 | — | — |
| Form E** | | 2 weeks | 99.04 | 100.03 | Free acid |
| (818159- | 60° C./close | 4 weeks | 98.89 | 99.88 | Form E |
| 61-B) | | 8 weeks | 98.96 | 99.95 | |
| | 40° C./75% | 2 weeks | 99.08 | 100.07 | |
| | RH/open | 4 weeks | 99.00 | 99.99 | |
| | | 8 weeks | 99.03 | 100.02 | |
| Na salt | Initial | | 99.68 | — | — |
| Form A | 60 ° C./close | 2 weeks | 99.33 | 99.65 | Sodium salt |
| (818159- | | 4 weeks | 98.91 | 99.23 | Form A |
| 09-B) | | 8 weeks | 98.43 | 98.75 | |
| | 40° C./75% | 2 weeks | 99.71 | 100.03 | New form |
| | RH/open | 4 weeks | 99.69 | 100.02 | |
| | | 8 weeks | 99.71 | 100.03 | |
| K salt | | | 99.75 | — | — |
| Form C | Initial | | 99.57 | 99.82 | New diffraction |
| (818159- | 60° C./close | 2 weeks | 99.64 | 99.89 | peaks with |
| 10-B) | | 4 weeks | 99.67 | 99.92 | some |
| | | 8 weeks | 99.76 | 100.01 | diffraction |
| | 40° C./75% | 2 weeks | 99.75 | 100.00 | peaks |
| | RH/open | 4 weeks | 99.75 | 100.00 | disappeared |
| Arginine | | 8 weeks | 99.72 | — | — |
| salt | Initial | | 99.76 | 100.04 | Arginine salt |
| Form A | 60 ° C./close | 2 weeks | 99.73 | 100.01 | Form A |
| (818159- | | 4 weeks | 99.71 | 99.99 | |
| 11-B) | | 8 weeks | 99.59 | 99.87 | |
| | 40° C./75% | 2 weeks | 99.70 | 99.98 | |
| | RH/open | 4 weeks | 99.74 | 100.02 | |
| | | 8 weeks | | | |

*Minor Form E was observed after further comparison;
**Minor Form B was observed after further comparison.

TABLE 39
Purity profile of free acid Form B (818159-08-B)
before and after solid stability evaluation

| | | Area (%) | | | | | | |
|---|---|---|---|---|---|---|---|---|
| | | | 60° C. | | | 40° C./75% RH | | |
| | | | 2 | 4 | 8 | 2 | 4 | 8 |
| #Peak | RRT | Initial | weeks | weeks | weeks | weeks | weeks | weeks |
| 1 | 0.88 | 0.23 | 0.20 | 0.22 | 0.20 | 0.20 | 0.23 | 0.20 |
| 2 | 1.00 | 99.47 | 99.49 | 99.47 | 99.51 | 99.50 | 99.47 | 99.52 |
| 3 | 1.14 | 0.10 | 0.11 | 0.11 | 0.10 | 0.11 | 0.11 | 0.09 |
| 4 | 1.34 | 0.20 | 0.20 | 0.20 | 0.20 | 0.20 | 0.19 | 0.19 |

TABLE 40
Purity profile of free acid Form E (818159-61-B)
before and after solid stability evaluation

| | | Area (%) | | | | | | |
|---|---|---|---|---|---|---|---|---|
| | | | 60° C. | | | 40° C./75% RH | | |
| | | | 2 | 4 | 8 | 2 | 4 | 8 |
| #Peak | RRT | Initial | weeks | weeks | weeks | weeks | weeks | weeks |
| 1 | 0.88 | 0.31 | 0.32 | 0.38 | 0.34 | 0.33 | 0.35 | 0.32 |
| 2 | 1.00 | 99.01 | 99.04 | 98.89 | 98.96 | 99.08 | 99.00 | 99.03 |
| 3 | 1.14 | 0.16 | 0.16 | 0.17 | 0.14 | 0.15 | 0.15 | 0.13 |
| 4 | 1.28 | 0.26 | 0.23 | 0.26 | 0.23 | 0.18 | 0.23 | 0.20 |
| 5 | 1.33 | 0.25 | 0.25 | 0.29 | 0.27 | 0.25 | 0.27 | 0.27 |

TABLE 41
Purity profile of Na salt Form A (818159-09-B)
before and after solid stability evaluation

| | | Area (%) | | | | | | |
|---|---|---|---|---|---|---|---|---|
| | | | 60° C. | | | 40° C./75% RH | | |
| | | | 2 | 4 | 8 | 2 | 4 | 8 |
| #Peak | RRT | Initial | weeks | weeks | weeks | weeks | weeks | weeks |
| 1 | 0.29 | — | — | 0.07 | 0.11 | — | — | — |
| 2 | 0.58 | — | 0.10 | 0.16 | 0.25 | — | — | — |
| 3 | 0.63 | — | — | 0.05 | 0.12 | — | — | — |
| 4 | 0.79 | — | 0.07 | 0.13 | 0.19 | — | — | — |
| 5 | 0.81 | — | 0.10 | 0.20 | 0.35 | — | — | — |
| 6 | 0.86 | — | — | 0.06 | 0.06 | — | — | — |
| 7 | 0.89 | 0.05 | 0.08 | 0.08 | — | 0.04 | 0.07 | 0.05 |
| 8 | 0.91 | — | 0.05 | 0.10 | 0.21 | — | — | — |
| 9 | 0.95 | — | — | 0.05 | 0.09 | — | — | — |
| 10 | 1.00 | 99.68 | 99.33 | 98.91 | 98.43 | 99.71 | 99.69 | 99.71 |
| 11 | 1.14 | 0.05 | 0.05 | — | — | 0.03 | — | — |
| 12 | 1.34 | 0.22 | 0.22 | 0.20 | 0.20 | 0.23 | 0.23 | 0.24 |

TABLE 42
Purity profile of K salt Form C (818159-10-B)
before and after solid stability evaluation

| | | Area (%) | | | | | | |
|---|---|---|---|---|---|---|---|---|
| | | | 60° C. | | | 40° C./75% RH | | |
| | | | 2 | 4 | 8 | 2 | 4 | 8 |
| #Peak | RRT | Initial | weeks | weeks | weeks | weeks | weeks | weeks |
| 1 | 0.88 | — | 0.10 | 0.08 | 0.09 | — | — | — |
| 2 | 1.00 | 99.75 | 99.57 | 99.64 | 99.67 | 99.76 | 99.75 | 99.75 |
| 3 | 1.14 | — | 0.07 | 0.04 | — | — | — | — |
| 4 | 1.34 | 0.25 | 0.26 | 0.24 | 0.24 | 0.24 | 0.25 | 0.25 |

TABLE 43
Purity profile of arginine salt Form A (818159-11-B)
before and after solid stability evaluation

| | | Area (%) | | | | | | |
|---|---|---|---|---|---|---|---|---|
| | | | 60° C. | | | 40° C./75% RH | | |
| | | | 2 | 4 | 8 | 2 | 4 | 8 |
| #Peak | RRT | Initial | weeks | weeks | weeks | weeks | weeks | weeks |
| 1 | 0.81 | — | — | — | 0.05 | — | — | — |
| 2 | 0.88 | 0.07 | 0.06 | 0.09 | 0.06 | 0.14 | 0.11 | 0.07 |
| 3 | 1.00 | 99.72 | 99.76 | 99.73 | 99.71 | 99.59 | 99.70 | 99.74 |
| 4 | 1.14 | — | — | — | — | 0.06 | — | — |
| 5 | 1.34 | 0.20 | 0.18 | 0.18 | 0.18 | 0.22 | 0.19 | 0.19 |

### Example 7: Evaluation of Compound 2 on PPARα/PPARγ Activity with Luciferase Reporter System

HEK293T cell culture followed ATCC culture guides. Experiments were performed when cells were in the exponential phase of growth. Total 6×10⁶ cells were seeded into a 60 mm cell culture dish and cultured overnight in 37° C. and 5% CO₂. Lipofectamine® 3000, the transfection reagent, was mixed with plasmid combination (a mixture of pGL4.35 [luc2P/9XGAL4 UAS/Hygro], pBIND-RXRα, and pBIND-PPPARα or a mixture of pGL4.35 [luc2P/9XGAL4 UAS/Hygro], pBIND-RXRα, and pBIND-PPPARγ), and then added to the dish. After incubated for 5 hours at 37° C. and 5% CO₂, the cells was trypsinized and seeded into a 384-well assay plate, followed by incubation with the test compound at the serial concentrations for overnight in 37° C. and 5% CO₂. At the following day, Cells were lyzed and luciferase was activated with the Steady-Glo™ Luciferase Assay System. Luminescence signals from the luciferase assay were measured by Envision HTS/2105. Since peroxisome proliferator-activated transcription regulates the expression of luciferase, the agonist activities of the test compounds can be qualified by the luminescence intensity. The EC₅₀ value of test compounds was calculated for the PPARα/γ agonistic potency by using Graphpad 8.0, and the results were shown in Table 44. Selectivity of compounds towards PPARα or PPARγ was expressed as PPARγ EC₅₀/PPARα EC₅₀.

TABLE 44
Results of PPARα/γ agonism testing

| Compound ID | PPARα EC₅₀ (nM) | PPARγ EC₅₀ (nM) | Selectivity |
|---|---|---|---|
| Aleglitazar | 3.14 | 3.36 | 1.07 |
| Compound 2 | 2.52 | 3.81 | 1.51 |

*Selectivity = PPARγ EC₅₀ (nM)/PPARα EC₅₀ (nM)

### Example 8: The Effect of Compound 2 on the Rat Model of Hyperlipidemia Induced by High Cholesterol Diet

### 8.1 Experimental Materials:

30 male Sprague-Dawley rats aged 6-8 weeks; source: SPF (Beijing) Biotechnology Co., Ltd.; animal certificate number: 110324201104469613.

### 8.2 Experimental Methods:

8.2.1 High cholesterol diet (ASHF4) was used to induce an animal model of hyperlipidemia in the SD rats.

8.2.2 The male SD rats were fed with high cholesterol diet (ASHF4, Dyet, China) for 14 days. On the day before the start of the administration (Day 0), the animals were divided into 5 groups based on body weights and serum indicators and were continually fed with the high-cholesterol diet. The treatment groups were orally dosed with either compound or vehicle while continuing the high-cholesterol diet for a total of 1 week. The animals were weighed daily before the treatment, and the compounds were given at 9:00-9:30 in the morning based on the body weight of the day. The specific grouping and dosing regimen were shown in Table 45.

TABLE 45
Grouping and dosing of the experimental animals

| | | | Dosing | | Dosing | Num- |
|---|---|---|---|---|---|---|
| | | Dosage | volume | Dosing | fre- | bers of |
| Group | Drugs | (mg/kg) | (mL/kg) | route | quency | Animals |
| 1 | High | — | 5 | Oral | QDx7 | 6 |
| | Cholesterol | | | | days | |
| | Diet + | | | | | |
| | Vehicle | | | | | |
| 2 | High | 0.2 | 5 | Oral | QDx7 | 6 |
| | Cholesterol | | | | days | |
| | Diet + | | | | | |
| | Aleglitazar | | | | | |
| 3 | High | 0.6 | 5 | Oral | QDx7 | 6 |
| | Cholesterol | | | | days | |
| | Diet + | | | | | |
| | Aleglitazar | | | | | |
| 4 | High | 0.2 | 5 | Oral | QDx7 | 6 |
| | Cholesterol | | | | days | |
| | Diet + | | | | | |
| | Compound 2 | | | | | |
| 5 | High | 0.6 | 5 | Oral | QDx7 | 6 |
| | Cholesterol | | | | days | |
| | Diet + | | | | | |
| | Compound 2 | | | | | |

Formulation. The formulation was prepared twice weekly. 1. Vehicle: 0.5% sodium carboxymethyl cellulose. 5 g sodium carboxymethyl cellulose was added into 900 ml ddH2O and stirred until dissolved completely, then filled into 1000 ml with ddH2O. 2. Working solution for 0.6 mg/kg administration: 0.12 mg/ml working solution. 12 mg compound was added into 100 ml of 0.5% sodium carboxymethyl cellulose, and then vortexed until well suspended. 3. Working solution for 0.2 mg/kg administration: 0.04 mg/ml working solution. 30 ml of 0.12 mg/ml compound solution was mixed with 60 ml of 0.5% sodium carboxymethyl cellulose, and then vortexed until well suspended.

8.2.3 Animal blood was collected, and serum was separated for the analysis of serum lipid indicators on the day before the treatment and at the end of the last day for the experiment.

8.2.4 Serum indicators of triglyceride (TG) and free fatty acid (NEFA) were determined by an automatic blood biochemical analyzer.

### 8.3 Results:

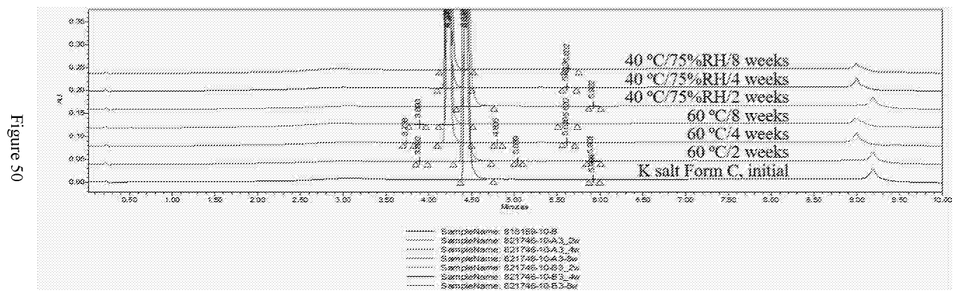

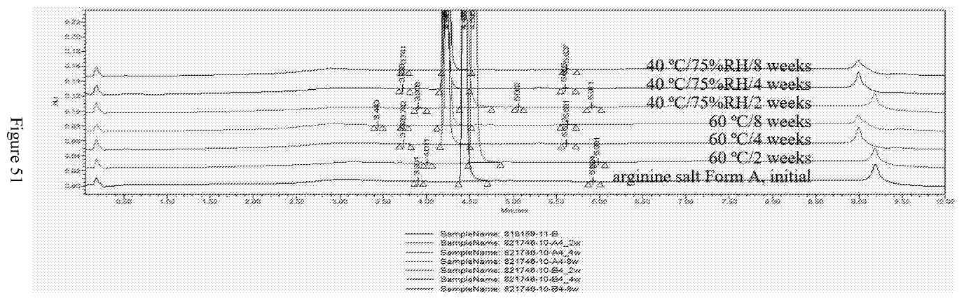

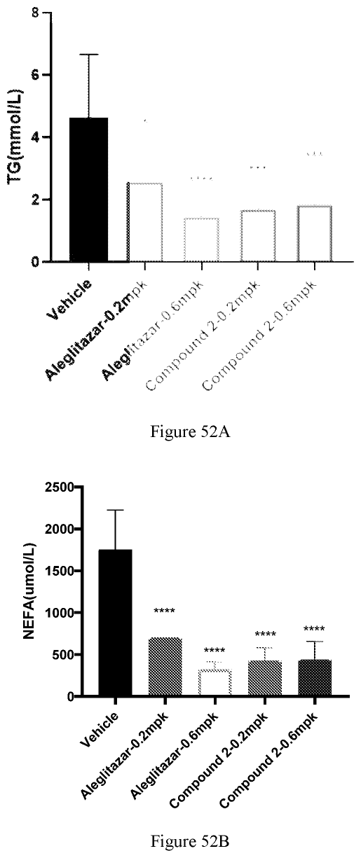

Serum lipid analysis showed that, after 7 days of treatment (Day 8), when compared with the vehicle group, Aleglitazar, and Compound 2 can all significantly reduce serum TG and NEFA levels at dosage levels of both 0.2 mg/kg and 0.6 mg/kg. The animal serum TG and NEFA were shown in FIGS. 52A and 52B, and their numerical values were shown in Table 46 and 47, respectively. During the experiment, there were no abnormal clinical observations. For comparative purposes, the GraphPad 8.0 software package was used to carry out T-test statistical analysis on the results of each compound at a dosage of 0.2 mg/kg. At dose level of 0.2 mg/kg, Compound 2 can significantly reduce TG and NEFA (P<0.05), when compared with Aleglitazar.

TABLE 46
Changes of serum TG in each group (mean ± standard deviation)

| | | TG (mmol/L) | |
|---|---|---|---|
| Group | Dosing Regimen | Day 0 | Day 8 |
| 1 | High Cholesterol Diet + Vehicle | 4.00 ± 1.75 | 4.63 ± 2.02 |
| 2 | High Cholesterol Diet + Aleglitazar | 4.24 ± 1.18 | 2.86 ± 0.86* |
| 3 | High Cholesterol Diet + Aleglitazar | 4.09 ± 2.17 | 1.47 ± 0.46**** |
| 4 | High Cholesterol Diet + Compound 2¹ | 4.27 ± 1.61 | 1.71 ± 0.72*** |
| 5 | High Cholesterol Diet + Compound 2 | 4.11 ± 1.10 | 1.86 ± 0.99*** |

Note:
Each group of data was analyzed by GraphPad 8.0 software package, and the statistical method is One-way ANOVA. Compared with the first group,
*p < 0.05,
**p < 0.01,
***p < 0.001,
****p < 0.0001.
¹TG of Group 4 (compound 2, 0.2 mg/kg) was statistically significantly lower (p < 0.05) when compared with that of Group 2 (Aleglitazar, 0.2 mg/kg)

TABLE 47
Changes of serum NEFA in each group (mean ± standard deviation)

| | | NEFA (umol/L) |
|---|---|---|
| Group | Dosing Regimen | Day 8 |
| 1 | High Cholesterol Diet + Vehicle | 1753.50 ± 472.12 |
| 2 | High Cholesterol Diet + Aleglitazar | 706.00 ± 190.15 |
| 3 | High Cholesterol Diet + Aleglitazar | 327.83 ± 90.73 |
| 4 | High Cholesterol Diet + Compound 2¹ | 431.50 ± 151.63 |
| 5 | High Cholesterol Diet + Compound 2 | 444.33 ± 119.12 |

Note:
Each group of data was analyzed by GraphPad 8.0 software package, and the statistical method is One-way ANOVA. Compared with the first group,
****p < 0.0001.
¹NEFA of Groups 4 (compound 2, 0.2 mg/kg) was statistically significantly lower (p < 0.05) when compared with that of Group 2 (Aleglitazar, 0.2 mg/kg).

### Example 9: Effect of 7-Day Repeated Oral Gavage on the Body Weights of ICR Mice

### 9.1 Experimental Materials:

50 male ICR mice aged 6-8 weeks; source: Laboratory Animal Business Department, Shanghai Institute of Planned Parenthood Research.

### 9.2 Methods:

After 3 days of acclimatization, ICR mice were grouped according to their body weights. The day of grouping is designated as Day 0. After grouping, they were dosed with either vehicle or compound by oral gavage for 7 consecutive days, once a day. The dosage and the grouping were shown in Table 48. The animals were weighed and recorded daily.

TABLE 48
Grouping and dosing of the experimental animals

| | | | Dosing | Dosing | Concen- | Number |
|---|---|---|---|---|---|---|
| | | Dosage | fre- | volume | tration | of |
| Group | Test agent | (mg/kg) | quency | (ml/kg) | (mg/mL) | Animals |
| 1 | Vehicle^{a} | 0 | QDx7 | 10 | 0 | 10 |
| | | | days | | | |
| 2 | Aleglitazar | 0.2 | QDx7 | 10 | 0.02 | 10 |
| | | | days | | | |
| 3 | Aleglitazar | 1 | QDx7 | 10 | 0.1 | 10 |
| | | | days | | | |
| 4 | Compound 2 | 0.2 | QDx7 | 10 | 0.02 | 10 |
| | | | days | | | |
| 5 | Compound 2 | 1 | QDx7 | 10 | 0.1 | 10 |
| | | | days | | | |

^{a}0.5% sodium carboxymethyl cellulose aqueous solution

Formulation. The formulation was prepared twice weekly. 1. Vehicle: 0.5% sodium carboxymethyl cellulose. 2.5 g sodium carboxymethyl cellulose was weighed and mixed with 500 ml ddH₂O until dissolved completely. 2. Working solution for 1 mg/kg administration: 0.1 mg/ml working solution. 3 mg of compound was added into 30 ml of 0.5% sodium carboxymethyl cellulose, and then vortexed until well suspended. 3. Working solution for 0.2 mg/kg administration: 0.02 mg/ml working solution. 6 ml of 0.1 mg/ml compound solution was mixed with 24 ml of 0.5% sodium carboxymethyl cellulose, and then stirred until well suspended.

### 9.3 Results:

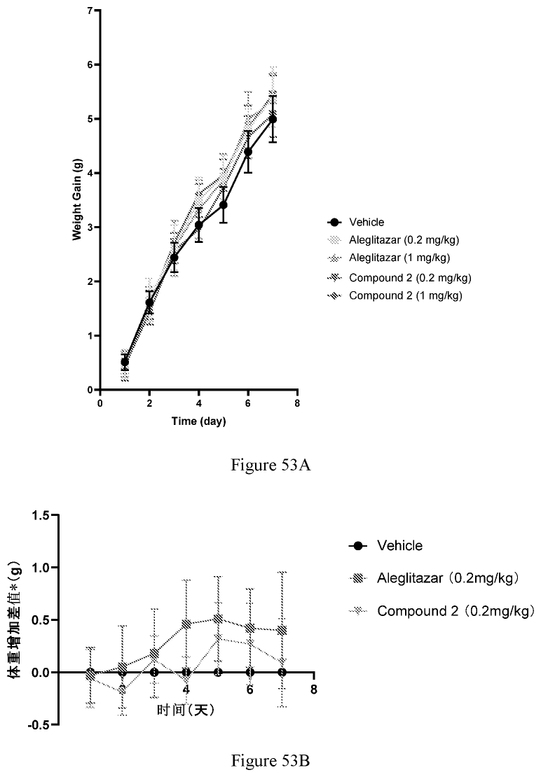

The effects of the compounds on the body weight changes of ICR mice were shown in Table 49 and FIG. 53A. During the experiment, the body weights of the animals gradually increased over time. The daily average weight gain of Aleglitazar groups at a dosage of 0.2 mg/kg (Day 4 and Day 5) and 1 mg/kg (Day 6) was significantly higher than that of the vehicle group. Compared with the vehicle group, the daily weight gain of Compound 2 group at a low dosage of 0.2 mg/kg had no significant difference throughout the study. The average daily weight gain of Compound 2 group at a high dosage of 1 mg/kg (Day 4 and 5) was significantly higher than that of the vehicle group. The full data was shown in Table 49. For the comparative purpose, the net weight gain of the treatment, which was derived from subtracting the average weight gain of the vehicle group from that of each treatment group, was calculated and shown in FIG. 53B.

TABLE 49
Changes in body weight gain of each group of animals compared with Day 0

| Compound | Weight Gain (g) n = 10 | | | | | | |
|---|---|---|---|---|---|---|---|
| ID | Day 1 | Day 2 | Day 3 | Day 4 | Day 5 | Day 6 | Day 7 |
| Vehicle | 0.51 ± 0.14 | 1.61 ± 0.2 | 2.44 ± 0.24 | 3.04 ± 0.31 | 3.4 ± 0.33 | 4.4 ± 0.38 | 5.0 ± 0.43 |
| Aleglitazar | 0.48 ± 0.27 | 1.66 ± 0.39 | 2.62 ± 0.42 | 3.50 ± 0.42 * | 3.92 ± 0.4 * | 4.81 ± 0.37 | 5.39 ± 0.56 |
| (0.2 mg/kg) | | | | | | | |
| Aleglitazar | 0.5 ± 0.2 | 1.64 ± 0.26 | 2.61 ± 0.51 | 3.33 ± 0.54 | 3.86 ± 0.49 | 4.98 ± 0.52 * | 5.34 ± 0.50 |
| (1 mg/kg) | | | | | | | |
| Compound 2 | 0.45 ± 0.28 | 1.42 ± 0.22 | 2.56 ± 0.23 | 2.96 ± 0.23 | 3.73 ± 0.34 | 4.66 ± 0.39 | 5.08 ± 0.42 |
| (0.2 mg/kg) | | | | | | | |
| Compound 2 | 0.4 ± 0.17 | 1.57 ± 0.26 | 2.71 ± 0.19 | 3.61 ± 0.19** | 3.96 ± 0.29 * | 4.84 ± 0.4 | 5.44 ± 0.36 |
| (1 mg/kg) | | | | | | | |

Note:
Each group of data was analyzed by GraphPad 8.0 software package, and the statistical method is One-way ANOVA.
Compared with vehicle,
* p < 0.05,
**p < 0.01,
*** p < 0.001,
**** p < 0.0001.

### Example 10: The Pharmacodynamics Study of Compound 2 on the db/db Type 2 Diabetes Model

### 10.1 Experimental Materials:

| Species | db/db mice | wild mice |
|---|---|---|
| Grade | SPF animal | SPF animal |
| Age when ordered | 6 weeks | 6 weeks |
| Age at the beginning of | 8 weeks | 8 weeks |
| the experiment | | |
| Weight range | 35~45 g | 18~25 g |
| Gender | Male | Male |
| Supplier | Jiangsu | Jiangsu |
| | GemPharmatech, | GemPharmatech, |
| | Co., Ltd. | Co., Ltd. |
| Address of the supplier | Jiangsu, China | Jiangsu, China |
| Identification method | Kept a mouse | Kept a mouse |
| | in a single | in a single |
| | numbered cage | numbered cage |
| Number of ordered | 55 | 8 |
| animals | | |
| Number of used animals | 45 | 6 |

### 10.2 Experimental Methods:

10.2.1 Grouping in the experiment: 6 Wild mice were used as the control group (Group 1). 45 Db/db mice were evenly divided into 5 groups before the initiation of treatment based on the body weights, serum triglyceride (TG) levels, and random blood glucose levels.

10.2.2 Formulation. The formulation was prepared twice weekly. 1. Vehicle: 0.5% sodium carboxymethyl cellulose. 2.5 g sodium carboxymethyl cellulose was weighed and mixed with 500 ml ddH2O until dissolved completely. 2. Working solution for 1 mg/kg administration: 0.1 mg/ml working solution. 3 mg of compound was added into 30 ml of 0.5% sodium carboxymethyl cellulose, and then vortexed until well suspended. 3. Working solution for 0.2 mg/kg administration: 0.02 mg/ml working solution. 6 ml of 0.1 mg/ml compound solution was mixed with 24 ml of 0.5% sodium carboxymethyl cellulose, and then stirred until well suspended. 4. Working solution for 0.05 mg/kg administration: 0.005 mg/ml working solution. 6 ml of 0.02 mg/ml compound solution was mixed with 18 ml of 0.5% sodium carboxymethyl cellulose, and then stirred until well suspended.

10.2.3 Dosing: The animals were orally dosed according to their grouping: Vehicle (Group 2), Aleglitazar at the dosage level of 0.2 mg/kg (Group 3), and Compound 2 at dosage levels of either 0.05 mg/kg (Group 4), 0.2 mg/kg (Group 5), or 1 mg/kg (Group 6). All animals were weighed daily before each dosing and treated daily for 14 consecutive days.

10.2.4 The study results include daily body weights throughout the study; serum TG levels before dosing at the 6^{th} and 12^{th} day of the study; the random blood glucose levels before dosing at the 7^{th} and 14^{th} day of the study; and results from oral glucose tolerance test (OGTT) performed at the 14^{th} day of the study.

### 10.3 Data Analysis:

All the data was imported into an Excel file and presented as Mean±SEM. Graphpad Prism 7.0 software was used for data statistical analysis by One-way or Two-way ANOVA, with P<0.05 as the criterion of significant differences.

### 10.4 Experimental Results:

Both Aleglitazar and all groups of Compound 2 significantly decreased the level of lipid, free fatty acid and blood glucose, and significantly increased body weight when compared with vehicle group.

### 10.4.1 Animal's Body Weight

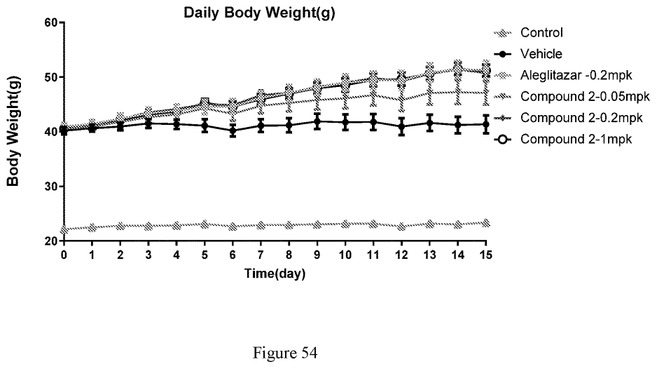

The body weight changes of db/db model animals treated with Aleglitazar and different dosages of Compound 2 are shown in FIG. 54. As shown in FIG. 54, animals' body weight in the dosage group of Aleglitazar (0.2 mg/kg) and dosage groups of Compound 2 (0.05 mg/kg, 0.2 mg/kg, 1 mg/kg) gradually increased over time during the experiment, and the average daily body weights (from Day 10 to Day 15) were all significantly higher than those of the vehicle group.

### 10.4.2 Animals' Blood Biochemical Indicators

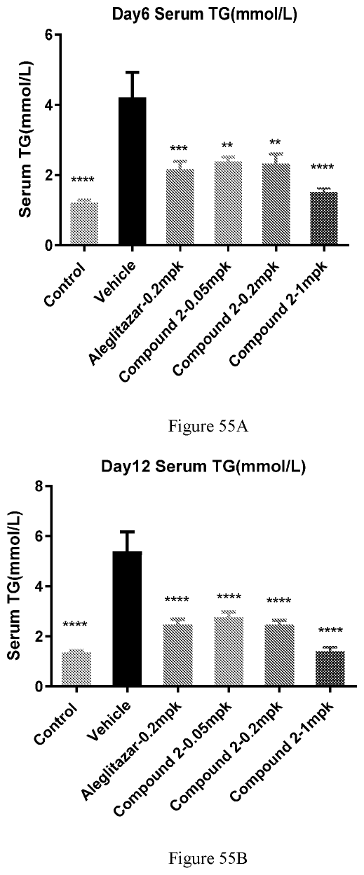

The animals' blood biochemical indicator TG was measured on Day 6 and Day 12, and the results were shown in FIGS. 55A and 55B. As shown in FIGS. 55A and 55B, the serum TG levels of groups treated with either Aleglitazar or Compound 2 at different dosages were significantly lower than those of the vehicle group on Day 6 and Day 12, where the largest effect was observed at Group 6 (Compound 2, 1 mg/kg).

### 10.4.3 Random Blood Glucose

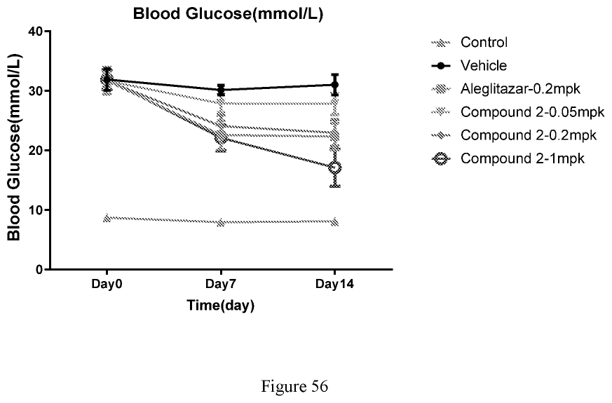

The effects of Aleglitazar and different dosages of Compound 2 on the random blood glucose of db/db model animals during the experimental period were shown in FIG. 56. Compared with the vehicle group, the random blood glucose levels of animals treated with either Aleglitazar or Compound 2 with different dosages were reduced on Day 7. Such reduction reached statistical significance at Group 3 (Aleglitazar, 0.2 mg/kg) and Group 6 (Compound 2, 1 mg/kg) but not in Groups 4 and 5 (Compound 2, 0.05 mg/kg & 0.2 mg/kg). On Day 14, more pronounced effect of blood glucose lowering was observed in Group 6 (Compound 2, 1 mg/kg) vs Group 2 (vehicle) when compared with Day 7, while Groups 3 and 5 (Aleglitazar, 0.2 mg/kg, and Compound 2, 0.2 mg/kg) showed similar effect. On the other hand, such effect in Group 3 (Compound 2, 0.05 mg/kg) still did not reach statistical significance. Thus Compound 2 had a slightly weaker effect on lowering blood glucose when compared with Aleglitazar (Table 50).

TABLE 50
The statistical difference of the blood glucose level between
each treatment group and the vehicle group

| | Blood Glucose (mmol/L) | | |
|---|---|---|---|
| Group | Day 0 | Day 7 | Day 14 |
| Aleglitazar (0.2 mpk) | p > 0.05 | p < 0.05 | p < 0.01 |
| Compound 2 (0.05 mpk) | p > 0.05 | p > 0.05 | p > 0.05 |
| Compound 2 (0.2 mpk) | p > 0.05 | p > 0.05 | p < 0.05 |
| Compound 2 (1 mpk) | p > 0.05 | p < 0.05 | p < 0.0001 |

### 10.4.4 Glucose Tolerance Test in Animals

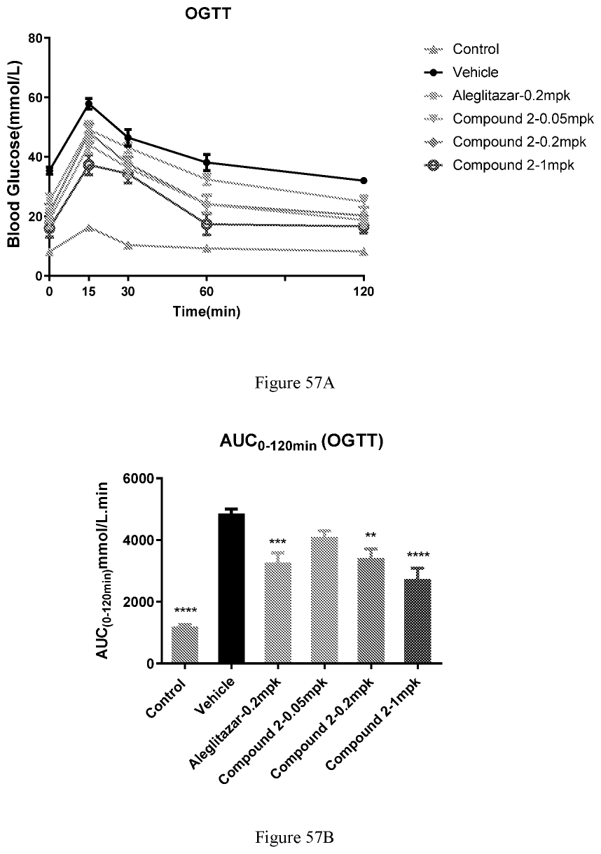

At the end of the experiment, oral glucose tolerance tests were carried out on the db/db animals treated by different compounds. Within 120 minutes after test, the blood glucose value at each time point and the area under the blood glucose-time curve were shown in FIGS. 57A and 57B. Compared with the vehicle group, the blood glucose levels of both Aleglitazar and Compound 2 at different dosages were significantly reduced at each time point. Among them, the AUC₀₋₁₂₀ min of the test product Aleglitazar (0.2 mg/kg, P<0.001) and Compound 2 (0.2 mg/kg, P<0.01 &1 mg/kg, P<0.0001) was significantly lower than that of the vehicle group. Besides, when compared with Aleglitazar, the AUC₀₋₁₂₀ min of Compound 2 at the same dose level (0.2 mg/kg) was higher, demonstrating a lower PPARγ activity. Furthermore, the results of Group 4 (Compound 2, 0.05 mg/kg) showed a trend of decrease but did not reach statistical significance at all time points when compared with the vehicle group.

TABLE 51
The statistical difference of the blood glucose level between each
OGTT treatment group and the vehicle group at each time point.

| | on-treatment period | | | | |
|---|---|---|---|---|---|
| Group | 0 min | 15 min | 30 min | 60 min | 120 min |
| Aleglitazar | p < 0.001 | p < 0.01 | p > 0.05 | p < 0.01 | p < 0.01 |
| (0.2 mpk) | | | | | |
| Compound | p > 0.05 | p > 0.05 | p > 0.05 | p > 0.05 | p > 0.05 |
| 2 (0.05 mpk) | | | | | |
| Compound | p < 0.01 | p > 0.05 | p > 0.05 | p < 0.01 | p < 0.05 |
| 2 (0.2 mpk) | | | | | |
| Compound | p < 0.0001 | p < 0.0001 | p < 0.05 | p < 0.0001 | p < 0.001 |
| 2 (1 mpk) | | | | | |

Note:
Statistical analysis of OGTT vs vehicle (Two-way ANOVA followed by Dunnett test by Prism Graphpad)

### 10.5 Discussion

In the disclosure, we obtained a novel compound with better α/γ activity, i.e., Compound 2.

In vitro transcription activity experiment showed that the EC₅₀ of the compound to activate PPARα and PPARγ pathways was at nanomolar level, indicating that Compound 2 had good in vitro biological activity. Compared with Aleglitazar, Compound 2 showed superior PPARα agonistic activity and weaker PPARγ agonistic ability.

Hyperlipidemia rat model experiment showed that Compound 2 could effectively and significantly reduce blood lipid levels in animals. In addition, Compound 2 at a low dose level had a better blood lipid-lowering effect than Aleglitazar at the corresponding concentration. Therefore, the results indicated that Compound 2 had better PPARα activity at a low dose level, leading to a better lipid-lowering effect.

ICR mouse bodyweight experiment showed that after treatment by low dose level of Compound 2, animals' body weight was comparable to that of the control group with no significant change. In contrast, the same dose level of Aleglitazar caused a significant increase in body weight. As weight increase is a well-known side effect of PPARγ, this demonstrated that, at a low dose level, the PPARγ activity of Compound 2 was weaker than that of Aleglitazar.

The study with db/db mice showed that Compound 2 could effectively reduce the blood glucose level and triglyceride content in type II diabetic mice. It indicated that Compound 2 can exhibit the in vivo biological effect of PPARγ to control blood glucose. Furthermore, Compound 2 at the same dose level can achieve a blood glucose-lowering effect similar to that of Aleglitazar. Therefore, the agonistic effect of Compound 2 on the PPARγ pathway is sufficient to achieve the regulation of glucose homeostasis.

### Example 11: Diabetic Nephropathy Pharmacodynamic Model

### 11.1 Experimental Methods

11.1.1 Animals: five-week-old wild type mice and db/db: BLKS male mice were purchased from Jiangsu GemPharmatech, Co., Ltd. The animals were housed in an SPF environment with 12-hour light/dark cycle. Housing temperature was maintained at 22-26° C. and humidity at 40%-60%. Mice were allowed to access food and water ad libitum. At the age of 6 weeks, db/db mice were anesthetized with 2.5% isopentane and subjected to uninephrectomy with removal of right kidney. Buprenorphine was applied after surgery.

11.1.2 Procedure. Two weeks after surgery, db/db mice were randomly assigned into 5 groups. Wild type mice were used for the control animals. Then total 5 animal groups were included in this study: Group1, control group with 6 animals dosed with vehicle; Group 2, vehicle groups with 10 animals dosed with vehicle; Group 3, Compound-low group with 10 animals dosed with Compound 2 at 0.1 mg/kg; Group 4, Compound-middle group with 10 animals orally dosed with Compound 2 at 0.3 mg/kg; and Group 5, Compound-high group with 10 animals dosed with Compound 2 at 1 mg/kg. Compounds were orally administrated once per day for 10 weeks.

11.1.3 Formulation. The formulation was prepared twice a week. 1. Vehicle: 0.5% sodium carboxymethyl cellulose. 2.5 g sodium carboxymethyl cellulose was weighed and mixed with 500 ml ddH₂O until dissolved completely. 2. Working solution for 1 mg/kg administration: 0.2 mg/ml working solution. 6 mg of compound was added into 30 ml of 0.5% sodium carboxymethyl cellulose, and then vortexed until well suspended. 3. Working solution for 0.3 mg/kg administration: 0.06 mg/ml working solution. 6 ml of 0.1 mg/ml compound solution was mixed with 14 ml of 0.5% sodium carboxymethyl cellulose, and then stirred until well suspended. 4. Working solution for 0.1 mg/kg administration: 0.02 mg/ml working solution. 2 ml of 0.2 mg/ml compound solution was mixed with 18 ml of 0.5% sodium carboxymethyl cellulose, and then stirred until well suspended.

At the 5^{th} and 9^{th} week after compound administration, mice were placed in metabolic cages for urine collection. Albumin level was measured for 24 hr albumin excretion calculation. At the 10^{th} week after treatment, the animals were sacrificed for kidney dissection. The kidney was fixed in 10% neutral buffed formalin, then paraffin-embedded for histopathological analysis. Glomerulosclerosis was assessed by evaluating glomerular basement membrane, mesangial expansion, nodular sclerosis and glomerulosclerosis. Severity was graded as follows: 0: normal; 1: Glomerular basement membrane thickening: isolated glomerular basement membrane thickening and only mild, nonspecific changes by light microscopy; 2: Mesangial expansion mild (IIa) or severe (IIb): glomeruli with mild or severe mesangial expansion but without nodular sclerosis or global glomerulosclerosis in more than 50% of glomeruli; 3: Nodular sclerosis: at least one glomerulus with nodular increase in mesangial matrix; 4: Advanced diabetic glomerulosclerosis: more than 50% global glomerulosclerosis with other clinical or pathological evidence that sclerosis is attributable to diabetic nephropathy. Renal tubular injury was scored as follows: 0: no obvious lesions; 1: up to 25% involvement of tubular lesion; 2: lesion in 25% to 50% of renal tubular; 3: 50% to 75% of renal tubular lesion and grade 4: >75% tubular lesion.

11.1.4 Data were presented as mean±SEM. Graphpad 8.0 software was used for statistical analysis. The difference between the values were analyzed with one-way ANOVA. The difference between histopathological score was analyzed with Kruskal-Wallis nonparameter test. All values were compared with the group 2.

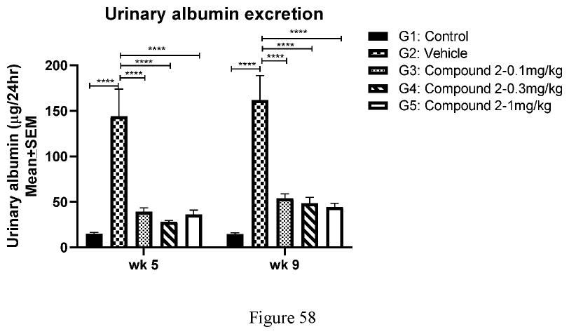

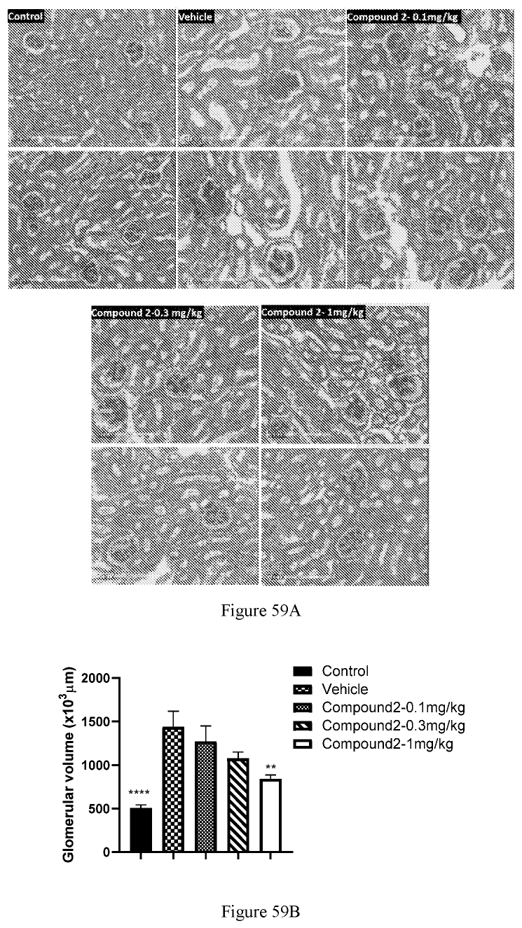

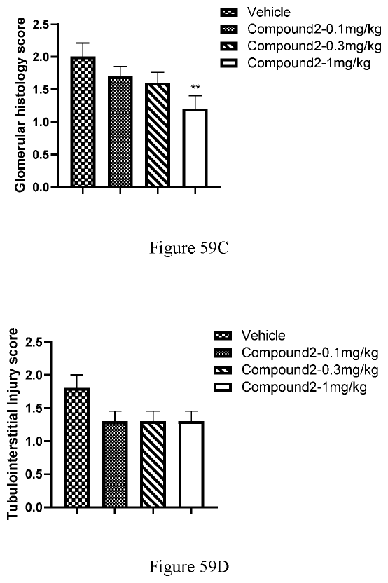

11.2 Results. 1. 24-hour urinary albumin. In the vehicle-treated uninephrectomied db/db mice, 24-hour urinary albumin increased by more than 10 folds compared with the control. Compound 2 significantly decreased 24-hour urinary albumin at the 5^{th} and 9^{th} weeks after compound administration. More than 50% reduction of urine albumin was achieved with Compound 2 treatment (FIG. 58). 2. Glomerulosclerosis. As shown in FIG. 59A, control animals had normal glomeruli appearance and glomerular volume. However, mesangial expansion, glomerular basement membrane thickening, and nodular sclerosis were observed in the vehicle-treated db/db animals. Compound 2 suppressed the glomerular damage and ameliorate the glomerular hypertrophy. Both histopathological score and glomerular volume were statistically significantly reduced after animal receiving high dose of Compound 2 (FIGS. 59B and 59C). 3. Renal tubular injury. Histopathological analysis showed control animals had normal tubular structure, but vehicle treated animals developed tubular dilation, basement membrane thickening/tubular atrophy and tubular cast. Compound 2 at all dosage levels improved the tubular injury at some extend (FIG. 59D).

### Example 12: Effect of Compound 2 on Improvement of Renal Injury in Rat Model of Unilateral Ureteral Obstruction

### 12.1 Experimental Methods:

12.1.1 Male SD rats weighing 240-260 g were housed in an SPF environment with 12-hour light/dark cycle. Housing temperature was maintained at 20-26° C. and humidity at 40%-60%. Rats were fed with a standard chow and allowed to access food and water ad libitum.

12.1.2 Formulation. The formulation was prepared twice a week. 1. Vehicle: 0.5% sodium carboxymethyl cellulose was prepared as described in 17.1.3. 2. 0.2 mg/ml solution of either Compound 2 or Aleglitazar. 6 mg of compound was added into 30 ml of 0.5% sodium carboxymethyl cellulose, and then vortexed until well suspended. 3. 0.02 mg/ml solution of either Compound 2 or Aleglitazar for the dosage of 0.2 mg/kg. 2 ml of 0.2 mg/ml solution was mixed with 18 ml of 0.5% sodium carboxymethyl cellulose, and then stirred until well suspended.

12.1.3 Procedure. After acclimation, animals are randomly assigned into the following groups: control group with 8 rats dosed with vehicle at 10 ml/kg; model group with 10 rats dosed with vehicle at 10 ml/kg, reference group with 10 animals dosed with Aleglitazar at 0.2 mg/kg; and compound group with 10 animals dosed with Compound 2 at 0.2 mg/kg. Vehicle or compounds were administrated by orally gavage daily. Unilateral ureteral obstruction was performed on the second day of compound treatment. Animals in model group, reference group and compound group underwent urethral ligation under isoflurane anesthesia one hour after receiving compound administration. A flank incision was cut to expose the left ureter, and a 4-0 surgical suture was used to perform two-point ligation to achieve urethral obstruction. Ureter was cut between the two ligation points. Animals in the control group were subjected to the same surgical treatment except the ligation and cut. After surgery, animals in each group continually received vehicle or compound for 12 more days, for a total of 14 treatment days.

Rats were sacrificed after 14 days' compound treatment. The obstructed kidney was weighed and collected for histology. The tissue samples were fixed with formalin, and then paraffin embedded. The embedded tissue was sectioned and stained with hematoxylin & eosin and Masson trichrome to assess kidney structure and fibrosis. Focal lesions included infract, tubular dilation, tubular obstruction and necrosis which were given score respectively between 0 and 4 as the percentage of the affected area of the biopsy (score 0: No, 1: <25%; 2: 25%-50%, 3: 50%-75%, 4: >75%). The severity of renal injury was evaluated by the total scores of the lesions. Kidney fibrosis was graded as 0-4 based on the percentage of area stained with Masson trichrome (score 0: No, 1: <25%; 2: 25%-50%, 3: 50%-75%, 4: >75%).

12.1.4 Data were presented as Mean±SEM. The multiple comparison was analyzed with the Kruskal-Wallis non-parameter test. The Dunn's test was used to compare the difference to the model group. A p value<0.05 was considered statistically significant.

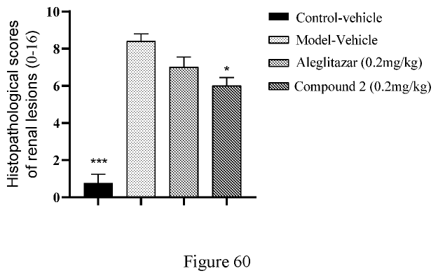

12.1.5 Results. When compared to the model group dosed with vehicle, Compound 2 at 0.2 mg/kg significantly improved the total score comprised of renal focal lesions, covering infract, tubular dilation, tubular obstruction, fibrosis, and necrosis (FIG. 60). Aleglitazar, the reference compound, on the other hand, did not achieve statistically significant improvement in total score at the same dose of 0.2 mg/kg. Therefore, compound 2 is superior to Aleglitazar in preventing and alleviating the renal injury caused by unilateral ureteral obstruction.

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

1.-101. (canceled)

102. A crystalline form B of Compound 2: 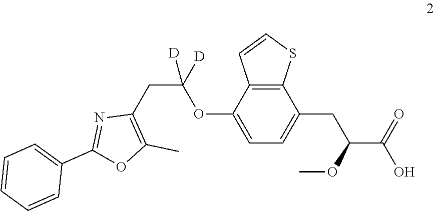 [SMILES: [2H]C([2H])(Cc1nc(-c2ccccc2)oc1C)Oc1ccc(C[C@H](OC)C(=O)O)c2sccc12]
characterized by an X-ray powder diffractogram (XRPD) pattern comprising one or more peaks at 8.16, 13.23 and 13.90 (±0.2° 2θ).

103. The crystalline form B of claim 102, wherein
a) the XRPD pattern further comprises one or more peaks at 4.94, 11.83, 14.97, 18.54 and 26.24 (±0.2° 2θ);
b) the XRPD pattern further comprises one or more peaks at 9.89, 19.74 and 20.92 (±0.2° 2θ);
c) the crystalline form B is characterized by an XRPD pattern comprising one or more peaks selected from the group consisting of:

| °2θ | °2θ | °2θ | °2θ | |
|---|---|---|---|---|
| 4.94 | 13.23 | 18.54 | 22.71 | |
| 8.16 | 13.90 | 19.74 | 25.35 | |
| 9.89 | 14.97 | 20.92 | 26.24 | |
| 11.83 | 16.29 | 21.63 | 28.55 | ; |

d) the crystalline form B is characterized by an XRPD pattern substantially as shown in FIG. 5A;
e) the crystalline form B is characterized by a differential scanning calorimetry (DSC) thermogram having an endotherm with a peak temperature of about 151.1° C.;
f) the crystalline form B is characterized by a DSC thermogram substantially as shown in FIG. 5B; or
g) the crystalline form B is an anhydrate.

104. A crystalline form C of Compound 2, characterized by an XRPD pattern comprising one or more peaks at 5.06, 10.09 and 20.24 (±0.2° 2θ).

105. The crystalline form C of claim 104, wherein
a) the XRPD pattern further comprises one or more peaks at 15.15, 16.18 and 16.75 (±0.2° 2θ);
b) the XRPD pattern further comprises one or more peaks at 7.99, 8.37, 11.71, 12.22, 12.87, 13.60 and 14.04 (±0.2° 2θ);
c) The crystalline form C is characterized by an XRPD pattern comprising one or more peaks selected from the group consisting of:

| °2θ | °2θ | °2θ | °2θ | |
|---|---|---|---|---|
| 5.06 | 12.22 | 16.18 | 25.89 | |
| 7.99 | 12.87 | 16.75 | 26.47 | |
| 8.37 | 13.60 | 19.43 | 30.56 | |
| 10.09 | 14.04 | 20.24 | 35.80 | |
| 11.71 | 15.15 | 20.96 | | ; |

d) the crystalline form C is characterized by an XRPD pattern substantially as shown in FIG. 6A;
e) the crystalline form C is characterized by a DSC thermogram having an endotherm with a peak temperature of about 149.0° C.;
f) the crystalline form C is characterized by a DSC thermogram substantially as shown in FIG. 6B; or
g) the crystalline form C is an anhydrate.

106. A crystalline form D of Compound 2, characterized by an XRPD pattern comprising one or more peaks at 5.03, 12.98, 15.91 and 21.58 (±0.2° 2θ).

107. The crystalline form D of claim 106, wherein
a) the XRPD pattern further comprises one or more peaks at 5.90, 9.21, 20.47 and 26.08 (±0.2° 2θ);
b) the XRPD pattern further comprises one or more peaks at 11.76, 15.04, 22.38 and 24.47 (±0.2° 2θ);
c) the crystalline form D is characterized by an XRPD pattern comprising one or more peaks selected from the group consisting of:

| °2θ | °2θ | °2θ | °2θ | |
|---|---|---|---|---|
| 5.03 | 11.76 | 15.91 | 22.38 | |
| 5.90 | 12.98 | 20.47 | 24.47 | |
| 9.21 | 15.04 | 21.58 | 26.08 | ; |

d) the crystalline form D is characterized by an XRPD pattern substantially as shown in FIG. 7; or
e) the crystalline form D is a hydrate.

108. A crystalline form E of Compound 2, characterized by an XRPD pattern comprising one or more peaks at 10.12, 11.97, 13.73 and 18.89 (±0.2° 2θ).

109. The crystalline form E of claim 108, wherein
a) the XRPD pattern further comprises one or more peaks at 5.06, 8.14, 13.02 and 14.19 (±0.2° 2θ);
b) the XRPD pattern further comprises one or more peaks at 14.87, 21.30, 22.33 and 26.23 (±0.2° 2θ);
c) the crystalline form E is characterized by an XRPD pattern comprising one or more peaks selected from the group consisting of:

| °2θ | °2θ | °2θ | °2θ | |
|---|---|---|---|---|
| 5.06 | 16.32 | 22.33 | 27.18 | |
| 8.14 | 18.00 | 23.01 | 27.68 | |
| 10.12 | 18.89 | 23.78 | 28.57 | |
| 11.97 | 19.36 | 24.23 | 29.51 | |
| 13.02 | 19.89 | 25.07 | 31.40 | |
| 13.73 | 20.31 | 25.40 | 32.47 | |
| 14.19 | 20.67 | 26.23 | 36.45 | |
| 14.87 | 21.30 | 26.74 | 39.05 | ; |

d) the crystalline form E is characterized by an XRPD pattern substantially as shown in FIG. 12; or
e) the crystalline form E is an anhydrate.

110. A crystalline form F of Compound 2, characterized by an XRPD pattern comprising one or two peaks at 15.50 and 21.23 (±0.2° 2θ).

111. The crystalline form F of claim 110, wherein
a) the XRPD pattern further comprises one or two peaks at 12.12 and 20.21 (±0.2° 2θ);
b) the crystalline form F is characterized by an XRPD pattern comprising one or more peaks selected from the group consisting of:

| °2θ | °2θ | °2θ | °2θ | |
|---|---|---|---|---|
| 12.12 | 15.50 | 20.21 | 21.23 | ; |

c) the crystalline form F is characterized by an XRPD pattern substantially as shown in FIG. 14A;
d) the crystalline form F is characterized by a DSC thermogram having endotherms with peak temperatures of about 111.2° C. and/or about 149.8° C.;
e) the crystalline form F is characterized by a DSC thermogram having an exothermal with a peak temperature of about 118.9° C.;
f) the crystalline form F is characterized by a DSC thermogram substantially as shown in FIG. 14B; or
g) the crystalline form F is a hydrate.

112. A crystalline form G of Compound 2, characterized by an XRPD pattern comprising one or more peaks at 9.67, 11.60, 12.90 and 14.43 (±0.2° 2θ).

113. The crystalline form G of claim 112, wherein
a) the XRPD pattern further comprises one or more peaks at 8.16, 13.69, 13.91, 16.15, 18.51, 18.93 and 20.47 (±0.2° 2θ);
b) the XRPD pattern further comprises one or more peaks at 4.95, 11.83, 13.22 and 19.84. (±0.2° 2θ);
c) the crystalline form G is characterized by an XRPD pattern comprising one or more peaks selected from the group consisting of:

| °2θ | °2θ | °2θ | °2θ | |
|---|---|---|---|---|
| 4.95 | 13.69 | 19.84 | 28.60 | |
| 8.16 | 13.91 | 20.47 | 29.94 | |
| 9.67 | 14.43 | 22.70 | 35.09 | |
| 11.60 | 14.84 | 25.28 | 35.93 | |
| 11.83 | 16.15 | 26.24 | | |
| 12.90 | 18.51 | 26.81 | | |
| 13.22 | 18.93 | 27.59 | | ; |

d) the crystalline form G is characterized by an XRPD pattern substantially as shown in FIG. 15A;
e) the crystalline form G is characterized by a DSC thermogram having endotherms with peak temperatures of about 72.4° C. and/or about 83.1° C.;
f) the crystalline form G is characterized by a DSC thermogram substantially as shown in FIG. 15B; or
g) the crystalline form G is a DMSO solvate.

114. A salt of Compound 2: 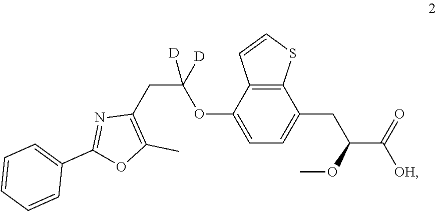 [SMILES: [2H]C([2H])(Cc1nc(-c2ccccc2)oc1C)Oc1ccc(C[C@H](OC)C(=O)O)c2sccc12]
wherein the salt of Compound 2 is selected from sodium salt, potassium salt, arginine salt, magnesium salt or tromethamine salt of Compound 2.

115. The salt of Compound 2 of claim 114, wherein the salt of Compound 2 is
a) in a crystalline form A of sodium salt of Compound 2
1) characterized by an XRPD pattern comprising one or more peaks at 4.09, 4.62 and 14.21 (±0.2° 2θ); optionally, wherein the XRPD pattern further comprises one or two peaks at 16.43 and 17.72 (±0.2° 2θ);
2) the crystalline form A of sodium salt is characterized by an XRPD pattern comprising one or more peaks selected from the group consisting of:

| °2θ | °2θ | °2θ | |
|---|---|---|---|
| 4.09 | 14.21 | 17.72 | |
| 4.62 | 16.43 | | ; |

3) the crystalline form A of sodium salt is characterized by an XRPD pattern substantially as shown in FIG. 24A;
4) the crystalline form A of sodium salt is characterized by a DSC thermogram having endotherms with peak temperatures of about 163.5° C. and/or about 204.0° C.;
5) the crystalline form A of sodium salt is characterized by a DSC thermogram having an exothermal with a peak temperature of about 166.5° C.; or
6) the crystalline form A of sodium salt is characterized by a DSC thermogram substantially as shown in FIG. 24B;
b) in a crystalline form C of potassium salt of Compound 2
1) characterized by an XRPD pattern comprising one or more peaks at 12.91, 14.96 and 21.23 (±0.2° 2θ); optionally the XRPD pattern further comprises one or more peaks at 14.12, 20.68, 25.17 and 26.46 (±0.2° 2θ); optionally the XRPD pattern further comprises one or more peaks at 3.92, 18.08, 22.48, 24.70 and 25.81 (±0.2° 2θ);
2) the crystalline form C of potassium salt is characterized by an XRPD pattern comprising one or more peaks selected from the group consisting of:

| °2θ | °2θ | °2θ | °2θ | |
|---|---|---|---|---|
| 3.92 | 14.96 | 20.68 | 25.17 | |
| 6.90 | 16.05 | 21.23 | 25.81 | |
| 7.79 | 16.83 | 22.48 | 26.46 | |
| 12.91 | 18.08 | 23.28 | 30.84 | |
| 14.12 | 19.11 | 24.70 | | ; |

3) the crystalline form C of potassium salt is characterized by an XRPD pattern substantially as shown in FIG. 26A;
4) the crystalline form C of potassium salt is characterized by a DSC thermogram having endotherms with peak temperatures of about 108.1° C., about 140.2° C. and/or about 161.3° C.; or
5) the crystalline form C of potassium salt is characterized by a DSC thermogram substantially as shown in FIG. 26B;
c) in a crystalline form A of arginine salt of Compound 2
1) characterized by an XRPD pattern comprising one or more peaks at 12.93, 13.43, 18.21 and 19.49 (±0.2° 2θ); optionally the XRPD pattern further comprises one or more peaks at 10.91, 15.83, 19.14, 20.93, 21.15 and 22.02 (±0.2° 2θ); optionally the XRPD pattern further comprises one or more peaks at 11.34, 19.84, 20.45, 25.63 and 26.15 (±0.2° 2θ);
2) the crystalline form A of arginine salt is characterized by an XRPD pattern comprising one or more peaks selected from the group consisting of:

| °2θ | °2θ | °2θ | °2θ | |
|---|---|---|---|---|
| 7.25 | 16.94 | 21.15 | 27.24 | |
| 10.91 | 18.21 | 22.02 | 27.81 | |
| 11.34 | 19.14 | 23.46 | 29.67 | |
| 12.93 | 19.49 | 24.10 | 30.90 | |
| 13.43 | 19.84 | 25.22 | 33.07 | |
| 14.57 | 20.45 | 25.63 | | |
| 15.83 | 20.93 | 26.15 | | ; |

3) the crystalline form A of arginine salt is characterized by an XRPD pattern substantially as shown in FIG. 28A;
4) the crystalline form A of arginine salt is characterized by a DSC thermogram having an endotherm with a peak temperature of about 195.2° C.; or
5) the crystalline form A of arginine salt is characterized by a DSC thermogram substantially as shown in FIG. 28B;
d) in a crystalline form A of potassium salt of Compound 2
1) characterized by an XRPD pattern substantially as shown in the upper curve of FIG. 19A;
2) the crystalline form A of potassium salt is characterized by a DSC thermogram having endotherms with peak temperatures of about 91.1° C., about 135.8° C. and/or about 147.7° C.; or
3) the crystalline form A of potassium salt is characterized by a DSC thermogram substantially as shown in FIG. 19B;
e) in a crystalline form B of potassium salt of Compound 2 characterized by an XRPD pattern substantially as shown in the lower curve of FIG. 19A;
f) in a crystalline form A of magnesium salt of Compound 2
1) characterized by an XRPD pattern substantially as shown in FIG. 21A;
2) the crystalline form A of magnesium salt is characterized by a DSC thermogram having endotherms with peak temperatures of about 90.7° C., about 114.9° C., about 134.0° C. and/or about 154.2° C.; or
3) the crystalline form A of magnesium salt is characterized by a DSC thermogram substantially as shown in FIG. 21B;
or
g) in crystalline form A of tromethamine salt of Compound 2
1) characterized by an XRPD pattern substantially as shown in FIG. 22A;
2) the crystalline form A of tromethamine salt is characterized by a DSC thermogram having endotherms with peak temperatures of about 71.8° C., about 76.6° C., about 113.9° C. and/or about 213.1° C.; or
3) the crystalline form A of tromethamine salt is characterized by a DSC thermogram substantially as shown in FIG. 22B.

116. A pharmaceutical composition comprising Compound 2 and a pharmaceutically acceptable excipient, wherein Compound 2 is in the crystalline form B of claim 102.

117. The pharmaceutical composition of claim 116, wherein
a) at least 50%, 60%, 70%, 80%, 85%, 90%, 95%, 99%, 99.5%, 99.9% or 99.99% of Compound 2 is in crystalline form B;
b) at least 50%, 60%, 70%, 80%, 85%, 90%, 95%, 99%, 99.5%, 99.9% or 99.99% of Compound 2 is in crystalline form C;
c) at least 50%, 60%, 70%, 80%, 85%, 90%, 95%, 99%, 99.5%, 99.9% or 99.99% of Compound 2 is in crystalline form D;
d) at least 50%, 60%, 70%, 80%, 85%, 90%, 95%, 99%, 99.5%, 99.9% or 99.99% of Compound 2 is in crystalline form E;
e) at least 50%, 60%, 70%, 80%, 85%, 90%, 95%, 99%, 99.5%, 99.9% or 99.99% of Compound 2 is in crystalline form F; or
f) at least 50%, 60%, 70%, 80%, 85%, 90%, 95%, 99%, 99.5%, 99.9% or 99.99% of Compound 2 is in crystalline form G.

118. A pharmaceutical composition comprising a salt of Compound 2 of claim 114 and a pharmaceutically acceptable excipient.

119. The pharmaceutical composition of claim 118, wherein
a) at least 50%, 60%, 70%, 80%, 85%, 90%, 95%, 99%, 99.5%, 99.9% or 99.99% of the salt of Compound 2 is in the crystalline form A of sodium salt;
b) at least 50%, 60%, 70%, 80%, 85%, 90%, 95%, 99%, 99.5%, 99.9% or 99.99% of the salt of Compound 2 is in the crystalline form C of potassium salt;
c) at least 50%, 60%, 70%, 80%, 85%, 90%, 95%, 99%, 99.5%, 99.9% or 99.99% of the salt of Compound 2 is in the crystalline form A of arginine salt;
d) at least 50%, 60%, 70%, 80%, 85%, 90%, 95%, 99%, 99.5%, 99.9% or 99.99% of the salt of Compound 2 is in the crystalline form A of potassium salt;
e) at least 50%, 60%, 70%, 80%, 85%, 90%, 95%, 99%, 99.5%, 99.9% or 99.99% of the salt of Compound 2 is in the crystalline form B of potassium salt;
f) at least 50%, 60%, 70%, 80%, 85%, 90%, 95%, 99%, 99.5%, 99.9% or 99.99% of the salt of Compound 2 is in the crystalline form A of magnesium salt; or
g) at least 50%, 60%, 70%, 80%, 85%, 90%, 95%, 99%, 99.5%, 99.9% or 99.99% of the salt of Compound 2 is in the crystalline form A of tromethamine salt.

120. A method for
a) the treatment and/or prophylaxis of diseases which are modulated by PPARα and/or PPARγ agonists in a subject, or
b) modulating PPARα and/or PPARγ in a subject in need thereof,
comprising administering an effective amount of the crystalline form B of Compound 2 of claim 102 to the subject.

121. The method of claim 120, wherein the disease is diabetes, non-insulin dependent diabetes mellitus, elevated blood pressure, dyslipidemia, atherosclerotic diseases, metabolic syndrome, diabetic nephropathy, or renal injury; optionally the renal injury is caused by ureteral obstruction or unilateral ureteral obstruction.
